(19) United States
(12) Patent Application Publication
(10) Pub. No.: US 2025/0361518 A1
(43) Pub. Date: Nov. 27, 2025

(54) AUTOFLOWERING GENES

(71) Applicant: Phylos Bioscience, Inc., Portland, OR (US)

(72) Inventors: Erica Bakker, Portland, OR (US); Alisha Holloway, Portland, OR (US); Annie Poczynek, Portland, OR (US)

(73) Assignee: Phylos Bioscience, Inc., Portland, OR (US)

(21) Appl. No.: 18/866,040

(22) PCT Filed: May 13, 2023
(86) PCT No.: PCT/US2023/066976

§ 371 Date: Nov. 14, 2024

Related U.S. Application Data
(60) Provisional application No. 63/342,621, filed on May 16, 2022.

(51) Int. Cl. C12N 15/82; C07K 14/415; C12Q 1/6895

(52) U.S. Cl. CPC C12N 15/827; C07K 14/415; C12Q 1/6895; C12Q 2600/13; C12Q 2600/156

(57) ABSTRACT

Provided herein is the identification and markers and causal genes associated with day-neutral autoflowering in plants and their use in selecting plants, including Cannabis plants, having autoflowering activity. The markers are useful for breeding autoflowering plants by obtaining nucleic acids, detecting one or more markers that indicate autoflowering activity, and establishing plant lines having such characteristics. Also provided are methods of editing plants to establish plant lines having autoflowering allelic variations.

This application claims priority benefit to U.S. provisional application No. 63/342,621, filed May 16, 2022, the entire contents of which are hereby incorporated by reference.

### SEQUENCE LISTING REFERENCE

Pursuant to 37 CFR §§ 1.821-1.825, a Sequence Listing in the form of an ASCII-compliant text file (entitled “2003-2-P1_ST25_Sequence_Listing.txt” created on May 3, 2022 and 84.5 kilobytes in size), which will serve as both the paper copy required by 37 CFR § 1.821(c) and the computer readable form (CRF) required by 37 CFR § 1.821(e), is submitted concurrently with the Instant application. The entire contents of the Sequence Listing are incorporated herein by reference.

### BACKGROUND OF THE INVENTION

Autoflowering plant varieties, e.g., Cannabis autoflowering varieties, begin flowering based on age. This is opposed to photosensitive plant varieties, which begin flowering based on the ratio of light to dark hours in a day. Autoflowering plant varieties consequently flower at a defined number of days after seed germination and can be grown at any day length. Conversion of photosensitive germplasm to autoflower allows for plants to mature early, which results in avoidance of late season pathogen and pest damage that would reduce yield. It also allows farmers to stagger planting for a more prolonged harvest window to distribute labor over time. It further allows plants to grow during the off season (fall, spring) when photosensitive varieties might not flower and mature.

The most common way to create autoflowering varieties is the use of traditional methods of breeding that select for segregated traits over multiple generations. However, traditional breeding methods are laborious and time-consuming.

In Arabidopsis, The UPF2 gene (AT2G39260) forms a surveillance complex with UPF1 and UPF3, which is believed to activate nonsense-mediated decay (NMD) of mRNAs (Ohtani and Wachter 2019; Plant & Cell Physiology 60: 1953-1960). T-DNA mutants of UPF1 and UPF3 in Arabidopsis cause a delay in flowering time (Jung et al. 2020; The Plant Cell 32: 1081-1101). Mutants of UPF1, UPF2 and UPF3 in Arabidopsis display more severe developmental phenotypes when cultivated under the 16 hour photoperiod than under the 10 hour photoperiod (Shi et al. Journal of Integrative Plant Biology 54, no. 2 (2012)). In Arabidopsis, the NMD pathway is involved in the silencing of alternative splicing products of genes involved in the regulation of flowering time: GRP7 and GRP8, SOC1, and CCA1 (Filichkin et al. Genome Research 20.1 (2010); Schöning et al. The Plant Journal 52, no. 6 (2007); Schöning at al Nucleic Acids Research 36, no. 22 (2008); Shi at al. Journal of Integrative Plant Biology 54, no. 2 (2012); Song et al. The Plant Cell 21.4 (2009)). T-DNA insertion mutants of GRP7 and GRP8 resulted in delayed flowering in Arabidopsis (Steffen et al. Plant and Cell Physiology 60 (2019)) and mutants of CCA1 altered clock-regulated gene expression (Green and Tobin, Proceedings of the National Academy of Sciences 96.7 (1999)). SOC1 controls flowering and is required for CO to promote flowering. SOC1 and AGL24 up-regulate each other's expression (Lee and Lee, Journal of experimental botany 61.9 (2010)). The loss-of-function mutant of ag124 shows late flowering and the overexpression of AGL24 causes early flowering (Yu et al., Proceedings of the National Academy of Sciences 99.25 (2004)). As a result, the autoflowering phenotype could be caused by one or more mutations in or near UPF2 causing the gene to be lower expressed or which cause changes in the UPF1 and/or UPF3 binding sites in tissues and during time points where and when this gene is involved in regulation of flowering time.

In Arabidopsis, RAP2.7/TOE1 (AT2G28550) functions as a transcription factor, which is part of the APETALA2 (AP2) family. The AP2 family consists of AP2 and five transcription factors: TOE1, TOE2, TOE3, SCHLAFMÜTZE (SMZ), and SCHNARCHZAPFEN (SNZ). (Aukerman and Sakai 2003, Chen 2004, Schmid et al. 2003). All six AP2 family members are predicted targets of microRNA172 (miR172) (Jung et al. 2007). miR172 over-producing plants exhibit early flowering under both long days and short days (Jung et al. 2007). miR172 is part of a photoperiodic pathway independent of CO (Jung at al. 2007). miR172 production is activated by SPL15, which is repressed by miR152. miR152 production goes down by age and increases in sucrose, as a result SPL15 is no longer repressed and miR172 is being produced. miR172 represses RAP2-7/TOE1 transcription factors (Kinoshita and Richter 2020). TOE1 binds to the FT promoter near the CO-binding site, in addition TOE1 interacts with the LOV domain of FKF1 and likely interferes with the FKF1-CO interaction, resulting in the partial degradation of the CO protein in the afternoon to prevent premature flowering (Zhang et al. 2015). A T-DNA insertion knock-out mutant of TOE1 (toe1) flowered earlier (Jung et al. 2007), whereas overexpression of TOE1 caused late flowering (Aukerman and Sakai 2003). As a result, the autoflower phenotype could be caused by one or more mutations that would render RAP2-7/TOE1 non-functional due to a frameshift causing a premature stop codon, or that would reduce functionality through changes in or near miR172 or AP binding sites, or that would significantly reduce expression in tissues and during time points where this gene is involved in regulation of flowering time.

SbPRR37 is a central repressor in the sorghum flowering regulatory pathway that controls flowering in response to day length. Sorghum plants containing a non-functional, truncated version of SbPRR37 caused by early termination before the Response Regulatory domain flower independently of photoperiod (=autoflowering phenotype), whereas sorghum plants containing the full length functional version of SbPRR37 flowered in response to photoperiod (=photosensitive phenotype; Murphy, R. L. et al. (2011), Proceedings of the National Academy of Sciences, 108(39), pp. 16409-16474).

The invention described herein utilizes markers, and allelic variations of the PRR37, UPF2 and/or RAP2-7/TOE1 genes, for selecting autoflowering attributes, which solves the laborious and time-consuming issues of traditional breeding methods by providing Cannabis and other plant breeders with a specific and efficient method for creating autoflowering varieties.

### SUMMARY OF THE INVENTION

The present teachings relate to genes responsible for autoflowering in Cannabis. In an embodiment a transgenic Cannabis plant is provided, whose genome comprises a homozygous deletion of at least a portion of an endogenous PRR37 gene and wherein the Cannabis plant comprises autoflowering activity. In an embodiment, the endogenous PRR37 gene comprises a genomic nucleic acid sequence having at least 90% sequence identity to SEQ ID NO:224 or a protein coding nucleic acid sequence having at least 90% sequence identity to SEQ ID NO:211. In an embodiment, the homozygous deletion results in a truncated amino acid sequence of a PRR37 protein. In an embodiment, the homozygous deletion comprises a protein coding nucleic acid sequence having at least 90% sequence identity to SEQ ID NOs:206, 207, 208, 209, or 210; or amino acid sequences having at least 90% sequence identity to SEQ ID NOs:213, 214, 215, 216, or 217. In an embodiment, an isolated cell from the Cannabis plant is provided. In an embodiment, an isolated nucleic acid sequence encoding a deletion in a PRR37 gene from a Cannabis plant and is capable of conferring autoflowering activity is provided. In an embodiment, an endogenous PRR37 gene comprises a genomic nucleic acid sequence having at least 90% sequence identity to SEQ ID NO:224 or a protein coding nucleic acid sequence having at least 90% sequence identity to SEQ ID NO:211 is provided. In an embodiment, the deletion results in a truncated amino acid sequence of a PRR37 protein. In an embodiment, the homozygous deletion comprises a protein coding nucleic acid sequence having at least 90% sequence identity to SEQ ID NOs:206, 207, 208, 209, or 210; or amino acid sequences having at least 90% sequence identity to SEQ ID NOs:213, 214, 215, 216, or 217. In an embodiment, an isolated cell whose genome comprising the nucleic acid sequence encoding a deletion in a PRR37 gene is provided.

In another embodiment, a method of making a Cannabis plant conferring autoflowering activity is provided. The method comprises replacing an endogenous PRR37 gene from a Cannabis plant with the isolated nucleic acid encoding a deletion in a PRR37 gene. In an embodiment, the endogenous PRR37 gene comprises a genomic nucleic acid sequence having at least 90% sequence identity to SEQ ID NO:224 or a protein coding nucleic acid sequence having at least 90% sequence identity to SEQ ID NO:211. In an embodiment, the replacing comprises gene editing. In an embodiment, the gene editing comprises CRISPR technology.

In another embodiment, a method for selecting one or more autoflowering plants is provided. The method comprises i) obtaining nucleic acids from a sample plant or its germplasm; (ii) detecting one or more markers that indicate autoflowering activity, and (iii) indicating autoflowering activity. In an embodiment, the method further comprises selecting the one or more plants indicating autoflowering activity. In an embodiment, the selection comprises marker assisted selection. In an embodiment, the detecting comprises an oligonucleotide probe. In an embodiment, the marker comprises a polymorphism at position 26 of SEQ ID NO:225. In an embodiment, the marker comprises a G to T polymorphism at position 26 of SEQ ID NO:225. In an embodiment, the one or more markers comprises a truncated or deleted protein product of the endogenous PRR37 gene. In an embodiment, the endogenous PRR37 gene comprises a genomic nucleic acid sequence having at least 90% sequence identity to SEQ ID NO:224 or a protein coding nucleic acid sequence having at least 90% sequence identity to SEQ ID NO:211. In an embodiment, the method further comprises crossing the one or more plants comprising the indicated autoflowering activity to produce one or more F1 or additional progeny plants, wherein at least one of the F1 or additional progeny plants comprises the indicated autoflowering activity. In an embodiment, the crossing comprises selfing, sibling crossing, or backcrossing. In an embodiment, the at least one additional progeny plant comprising the indicated autoflowering activity is an F2-F7 progeny plant. In an embodiment, the selfing, sibling crossing, or backcrossing comprises marker-assisted selection. In an embodiment, the selfing, sibling crossing, or backcrossing comprises marker-assisted selection for at least two generations.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates a schematic representation of sequence fragments demonstrating the autoflowering causal polymorphism results in the loss of a canonical splice site (dark arrow) and the use of an alternative canonical splice site (light arrow) results in a preliminary stop codon. PS=photosensitive and AF=autoflowering.

### DETAILED DESCRIPTION OF THE INVENTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The present teachings relate generally to methods of producing autoflowering Cannabis varieties.

The terminology used in the disclosure herein is for the purpose of describing particular embodiments only and is not intended to limit the disclosure. As used in the description of the embodiments of the disclosure and the appended claims, the singular forms “a”. “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill. In the art to which this disclosure belongs.

### Definitions

The term “Abacus” as used herein refers to the Cannabis reference genome known as the Abacus reference genome (version CsaAba2).

The phrase “altering expression” refers to the production of gene product(s) in transgenic organisms in amounts or proportions that differ significantly from the amount of the gene product(s) produced by the corresponding wild-type organisms (i.e., expression is increased or decreased).

The term “amino acid” refers to an organic compound containing amino and carboxyl functional groups with side chains specific to each amino acid. An amino acid position refers to its position within a sequence of amino acids.

The term “autoflower” or “autoflowering” or “day-neutral” refers to a process, or plant possessing a process, wherein flowering of the plant is independent from a specific number of days experiencing light. A marker that indicates autoflowering activity is a marker that indicates whether a plant possesses an autoflowering phenotype.

The term “alternative nucleotide call” is a nucleotide polymorphism relative to a reference nucleotide for a SNP marker that is significantly associated with the causative SNP(s) that confer(s) an autoflowering phenotype.

The term “backcrossing” or “to backcross” refers to the crossing of an F1 hybrid with one of the original parents. A backcross is used to maintain the identity of one parent (species) and to incorporate a particular trait from a second parent (species). The best strategy is to cross the F1 hybrid back to the parent possessing the most desirable traits. Two or more generations of backcrossing may be necessary, but this is practical only if the desired characteristic or trait is present in the F1.

The term “beneficial” as used herein refers to an allele conferring an autoflowering phenotype.

The term “Cannabis” refers to plants of the genus Cannabis, including Cannabis sativa, and subspecies, Cannabis sativa indica, and Cannabis sativa ruderalis. Hemp is a type of Cannabis having low levels of tetrahydrocannabinol.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The terms “construct,” “plasmid,” “vector,” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present invention. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-88 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The term “cross”, “crossing”, “cross pollination” or “cross-breeding” refer to the process by which the pollen of one flower on one plant is applied (artificially or naturally) to the ovule (stigma) of a flower on another plant. Backcrossing is a type of cross in which a breeder repeatedly crosses hybrid progeny, for example a first generation hybrid (F1), back to one of the parents of the hybrid progeny. Backcrossing can be used to introduce one or more single locus conversions from one genetic background into another. Selfing is another type of cross in which pollen from one plant is directly placed onto the flower of the same plant. Sibling crossing is a type of cross between sibling plants, which can be either where plants being crossed share the same parents (i.e., a full sibling cross) or where plants being crossed share one of the same parents (i.e., a half sibling cross).

The term “detect” or “detecting” refers to any of a variety of methods for determining the presence of a nucleic acid.

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation. Northern blot, RT-PCR, Western blot, or in vitro, in situ, or in vivo protein activity assay(s).

The term “functional” as used herein refers to DNA or amino acid sequences which are of sufficient size and sequence to have the desired function (i.e., the ability to cause expression of a gene resulting in gene activity expected of the gene found in a reference genome, e.g., the Abacus reference genome).

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genetic modification” or “genetic alteration” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence. One type of gene modification may be gene silencing, which is a reduction or complete absence of gene expression.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leaves, stems, pollen, or cells that can be cultured into a whole plant.

The term “haplotype” refers to the genotype of a plant at a plurality of genetic loci, e.g., a combination of alleles or markers. Haplotype can refer to sequence polymorphisms at a particular locus, such as a single marker locus, or sequence polymorphisms at multiple loci along a chromosomal segment in a given genome. As used herein, a haplotype can be a nucleic acid region spanning two markers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “homozygous deletion” refers to the deletion of one or more complementary nucleotides.

The term “hybrid” refers to a variety or cultivar that is the result of a cross of plants of two different varieties. An exemplary hybrid would be a plant that is the result of a cross between an autoflowering plant and a photosensitive plant. A hybrid, as described here, can refer to plants that are genetically different at any particular loci. A hybrid can further include a plant that is a variety that has been bred to have at least one different characteristic from the parent, e.g., a progeny plant created from a cross between an autoflowering plant and a photosensitive plant wherein the hybrid progeny has at least one phenotypic characteristic that is different from one of the parent plants. “F1 hybrid” refers to the first generation hybrid, “F2 hybrid” the second generation hybrid, “F3 hybrid” the third generation, and so on. A hybrid refers to any progeny that is either produced or developed.

The term “inbreeding” refers to the production of offspring via the mating between relatives. The plants resulting from the inbreeding process are referred to herein as “inbred plants” or “inbreds.”

The term “introduced” refers to a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “offspring” refers to any plant resulting as progeny from a vegetative or sexual reproduction from one or more parent plants or descendants thereof. For instance, an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “percent sequence identity” or “sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The term “plant” refers to a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The terms “polynucleotide,” “polynucleotide sequence,” “nucleotide,” “nucleotide sequence,” “nucleic acid sequence,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their S-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. An “Isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “probe” or “nucleic acid probe” or “oligonucleotide probe” as used herein, is defined to be a collection of one or more nucleic acid fragments whose specific hybidization to a nucleic acid sample comprising a region of interest can be detected. The probe may be unlabeled or labeled as described below so that its binding to the target nucleic acid of interest can be detected. What “probe” refers to specifically is clear from the context in which the word is used. The probe may also be isolated nucleic acids immobilized on a solid surface (e.g., nitrocellulose, glass, quartz fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes described herein can be modified to a certain degree to produce probes that are “substantially identical” to the disclosed probes but retain the ability to specifically bind to (i.e., hybridize specifically to) the same targets or samples as the probe from which they were derived (see discussion above). Such modifications are specifically covered by reference to the individual probes described herein.

The term “promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

The term “photosensitive time course” as used herein generally refers to a time course taken that compares flowering times of a plant relative to plants having a flowering schedule determined by periods of light, in particular, natural daylight.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid. As used herein, the term “protein” is synonymous with the related terms “peptide” and “polypeptide.”

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. A “substantially homologous sequence” refers to variants of the disclosed sequences such as those that result from site-directed mutagenesis, as well as synthetically derived sequences. A substantially homologous sequence of the present invention also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present invention.

The term “target region” or “nucleic acid target” refers to a nucleotide sequence that resides at a specific chromosomal location. The “target region” or “nucleic acid target” is specifically recognized by a probe.

The term “transition” as used herein refers to the transition of a nucleotide at any specific genomic position with that of a different nucleotide.

The term “transgenic” refers to a plant, plant cell, tissue, organ, or material that comprises a gene or a genetic construct, comprising a “transgene” that has been transferred into the plant, plant cell, tissue, or organ by natural or artificial means.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis strains have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation, with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known strains of Cannabis are wind-pollinated and the fruit is an achene. Most strains of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced by Cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L.”, Pharmacology Biochemistry and Behavior 95 (4): 434-42, incorporated herein by reference). The two cannabinoids usually produced in greatest abundance are cannabidiol (CBD) and/or Δ9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

### Autoflower Markers and Haplotypes

Typically, sun-grown Cannabis is planted in spring, flowers when night periods exceed about 10-12 hours, and is ready to harvest in late autumn. Photoperiod refers to a plant's response to the amount of light and darkness to which it is exposed. Short-day or long-night plants, as obligate photoperiodic plants, will only begin flowering once the sunlight hours are reduced to a certain number, based on the seasonal changes of the earth's orbit or artificial replication thereof. Typically, short-day plants will flower when the day is less than 12 hours (i.e., the night is longer than 12 hours) regardless of plant age or size. In indoor growing operations, this photosensitivity allows for a precisely tailored plant cycle for continuous growing seasons with the stages of development being artificially controlled. Additionally, when outdoors, short-day plants can be fooled into flowering early (i.e., outside of the natural seasonal schedule) by being covered for at least 12 hours in a 24-hour period. Similarly, if exposed to more than 12 hours of light in a 24-hour period, short-day plants will not flower, so flowering may be delayed and/or a plant may be kept in a perpetual vegetative state (e.g., as a mother plant for clones and/or seeds).

Autoflowering or day-neutral plants, by contrast, will flower regardless of day or night length, based on various factors including plant maturity, total amount of light exposure, angle of the sun, degree-days, and root system containment. Indoor growing operations can therefore cause day-neutral plants to flower quickly or early based on the amount of light exposure, even running grow lights constantly. Conversely, this means that day-neutral plants may not be preserved in a vegetative state and will flower no matter if placed in perpetual darkness or light.

The present invention describes the discovery of novel autoflowering markers for plants, including Cannabis. Plants with the markers described herein exhibit an autoflowering phenotype. Thus, the autoflowering markers described herein allow for screening of plants exhibiting early autoflowering. Accordingly, the present invention describes a method for selecting one or more autoflowering plants, the method comprising i) obtaining nucleic acids from a sample plant or its germplasm; (ii) detecting one or more markers that indicate autoflowering activity, and (iii) indicating autoflowering activity. An embodiment further describes selecting the one or more plants indicating autoflowering activity. The use of marker-assisted selection in breeding activities is described below.

In an embodiment, the markers described in Table 1 can be used to select one or more plants having autoflowering activity. Table 1 describes 171 markers having high significance to plants exhibiting autoflowering activity, and lists the marker name, the respective p-value, the respective type indicative of the autoflowering phenotype (i.e., homozygous for the reference or alternative allele), the reference allele call, and the alternative allele call. In an embodiment, the one or more marker position comprises a polymorphism in the reference allele of the Abacus Cannabis reference genome on chromosome 1 relative to position 63,161,656; 63,308,184; 63,355,114; 63,422,002; 63,449,699; 63,589,885; 63,675,478; 63,765,361; 63,767,236; 63,775,211; 63,777,630; 63,833,581; 63,925,984; 63,930,893; 63,945,679; 64,035,782; 64,041,749; 64,187,259; 64,233,047; 64,238,617; 64,253,959; 64,254,725; 64,261,547; 64,262,905; 64,349,232; 64,363,968; 64,377,929; 64,515,399; 64,575,147; 64,663,448; 64,686,430; 64,879,585, 64,920,471; 65,004,163; 65,022,168; 65,181,429; 65,183,123; 65,220,358; 65,270,412; 65,423,973; 65,457,650; 65,479,355; 65,510,077; 65,533,197; 65,581,703; 65,586,925; 66,123,957; 66,213,077; 66,540,589; 66,925,020; 67,609,581; 67,695,735; 67,708,527, 67,711,595; 67,761,686; 67,780,949, 67,858,135; 67,892,254; 67,919,111; 67,972,467; 68,100,304; 68,184,751, 68,393,736; 68,451,268; 69,116,895; 69,243,942; 69,255,336; 69,275,241; 69,304,025; 69,469,022; 70,249,642; 70,580,989; 70,585,368; 70,587,829; 70,614,319; 70,614,532; 70,624,359; 70,686,503; 70,884,481; 71,067,519; 71,070,939; 71,359,028; 71,550,096; 71,671,694; 71,695,399; 71,718,071; 71,824,879; 71,858,474; 72,378,842; 72,454,019; 72,455,436; 72,743,748; 73,473,406; 73,517,405; 73,817,673; 73,826,184; 73,836,391; 73,911,833; 73,982,309; 74,787,289; 77,758,271; 78,122,009; 48,727,601; 63,267,403; 63,270,572; 63,358,922; 63,445,606; 63,542,841; 63,622,828; 63,721,208; 63,723,647; 64,003,743; 64,037,854; 65,019,322; 65,050,650; 65,137,864; 65,173,837; 65,181,428; 65,761,925; 65,886,304; 65,927,579; 65,933,598; 65,963,869; 65,985,313; 65,990,175; 66,001,667; 66,015,507; 66,099,050; 66,531,090; 66,665,268; 66,683,626; 66,740,867; 66,834,787; 66,983,293; 67,034,241; 67,129,334; 67,454,121; 87,498,547; 67,585,755; 67,602,283; 67,629,801; 67,903,472; 67,976,538; 68,446,452; 68,470,691; 68,493,804; 68,567,745; 68,887,689; 68,899,476; 68,932,932; 69,078,399; 69,415,301; 69,448,252; 69,452,673; 69,496,492; 69,561,200; 69,576,766; 69,803,046; 70,367,062; 71,980,891; 75,648,136; 74,962,881; 65,215,553; 65,870,980; 65,980,912; 65,129,138; 65,244,439; 65,470,698; 65,485,211; 65,572,130; or 65,601,780 as described in Table 1.

In an embodiment, the markers described in Table 1 can be used to select one or more plants having autoflowering activity, the markers described as being position 13 in the 25 nucleotide sequences as described in Table 7. Table 7 assigns sequence identifiers to the markers described in Table 1. The present invention thus describes markers signifying an autoflowering phenotype wherein the marker comprises a polymorphism at position 13 of any one or more of SEQ ID NOs:1-171, and Table 1 can be used to associate which polymorphisms at position 13 of SEQ ID NOs:1-171 are significantly correlating with an autoflowering phenotype. In an embodiment, position 13 of SEQ ID NO:1 Is a marker associated with autoflowering. The present invention accordingly provides that the marker comprises a polymorphism at position 13 of any one or more of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:86; SEQ ID NO:87; SEQ ID NO:88; SEQ ID NO:89; SEQ ID NO:90; SEQ ID NO:91; SEQ ID NO:92; SEQ ID NO:93; SEQ ID NO:94; SEQ ID NO:95; SEQ ID NO:96; SEQ ID NO:97; SEQ ID NO:98; SEQ ID NO:99; SEQ ID NO:100; SEQ ID NO:101; SEQ ID NO:102; SEQ ID NO: 103; SEQ ID NO:104; SEQ ID NO:105; SEQ ID NO:106; SEQ ID NO:107; SEQ ID NO:108; SEQ ID NO:109; SEQ ID NO:110; SEQ ID NO:111; SEQ ID NO:112; SEQ ID NO:113; SEQ ID NO:114; SEQ ID NO:115; SEQ ID NO:116; SEQ ID NO:117; SEQ ID NO:118; SEQ ID NO:119; SEQ ID NO:120; SEQ ID NO:121; SEQ ID NO:122; SEQ ID NO:123; SEQ ID NO:124; SEQ ID NO:125; SEQ ID NO:126; SEQ ID NO:127; SEQ ID NO:128; SEQ ID NO:129; SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; SEQ ID NO:144; SEQ ID NO:145; SEQ ID NO:146; SEQ ID NO:147; SEQ ID NO:148; SEQ ID NO:149; SEQ ID NO:150; SEQ ID NO:151; SEQ ID NO:152; SEQ ID NO:153; SEQ ID NO:154; SEQ ID NO:155; SEQ ID NO:156; SEQ ID NO:157; SEQ ID NO:158; SEQ ID NO:159; SEQ ID NO:160; SEQ ID NO:161; SEQ ID NO:162; SEQ ID NO:163; SEQ ID NO:164; SEQ ID NO:165; SEQ ID NO:166; SEQ ID NO:167; SEQ ID NO:168; SEQ ID NO:169; SEQ ID NO:170; or SEQ ID NO:171.

In an embodiment, the markers described in Table 2 can be used to select one or more plants having autoflowering activity. Table 2 describes 265 additional markers having high significance to plants exhibiting autoflowering activity, and lists the marker name, the respective p-value, the respective type indicative of the autoflowering phenotype (i.e., homozygous for the reference or alternative allele), the reference allele call, and the alternative allele call. In an embodiment, the one or more marker position comprises a polymorphism in the reference allele of the Abacus Cannabis reference genome on chromosome 1 relative to position 268,476; 3,326,542; 15,402,934; 16,672,487; 19,090,442; 20,962,173; 25,416,995; 25,975,749; 27,376,279; 27,463,437; 27,527,476; 30,742,977; 30,874,960; 30,883,438; 30,899,325; 31,017,608; 31,082,669; 31,164,922; 32,317,496; 32,459,479; 32,941,839; 33,407,180; 33,692,404; 33,809,865; 33,867,472; 33,882,304; 33,915,586; 34,104,715; 34,111,342; 34,236,079; 34,335,660; 34,390,673; 34,403,630; 34,443,652; 34,482,685; 34,490,939; 34,523,417; 34,780,632; 34,891,501; 35,311,416; 35,380,437; 35,484,450; 35,495,416; 35,510,063; 36,403,557; 37,068,689; 37,071,526; 37,179,593; 37,576,767; 37,674,639; 37,925,069; 37,927,201; 38,043,498; 38,175,429; 38,298,835; 38,498,502; 38,530,025; 38,544,151; 38,594,588; 38,844,471; 38,862,689; 39,073,782; 39,084,115; 39,097,992; 39,359,130; 39,383,118; 39,921,599; 40,830,255; 40,870,508; 40,958,538; 41,197,544; 41,307,507; 42,191,944; 42,229,455; 42,396,589; 42,412,816; 42,508,652; 42,603,366; 42,065,152; 43,047,034; 43,215,274; 43,355,502; 43,382,522; 43,904,143; 43,923,005; 44,236,127; 44,246,864; 44,262,185; 45,191,090; 45,516,981; 45,562,350; 45,563,891; 45,592,056; 45,693,190; 46,397,576; 46,405,726; 46,474,244; 47,604,285; 47,665,099; 47,672,379; 47,708,135; 48,380,340; 48,388,505; 48,757,508; 48,920,387; 50,082,232; 50,178,362; 50,220,108; 50,234,848; 50,877,604; 50,909,707; 50,914,980; 50,943,468; 51,285,462; 51,285,752; 51,585,800; 51,729,989; 51,745,672; 52,506,950; 52,549,792; 54,566,650; 55,366,336; 56,490,139; 56,660,721; 56,968,116; 57,308,692; 57,712,867; 60,822,892; 62,480,171; 63,128,832; 63,599,570; 63,714,224; 63,921,961; 64,341,255; 64,547,738; 65,036,575; 66,071,116; 66,631,011; 66,775,861; 66,784,085; 66,885,379; 67,272,033; 67,514,890; 67,535,229; 67,656,258; 68,551,248; 68,558,021; 68,562,883; 68,592,104; 68,721,246; 68,730,683; 69,003,698; 69,072,463; 69,236,641; 69,239,452; 69,305,092; 69,539,678; 69,545,637; 69,678,995; 70,364,873; 70,552,675; 70,696,508; 70,769,733; 71,191,901; 71,204,416; 71,213,884; 71,283,642; 71,464,643; 71,476,054; 71,716,668; 71,737,576; 71,840,991; 71,902,441, 72,043,845, 72,047,815, 72,220,564, 72,250,376; 72,251,358; 72,335,998; 72,515,564; 72,585,309; 72,690,334; 72,762,298; 72,786,344; 72,813,354; 72,856,290; 72,941,220; 73,173,850; 73,250,920; 73,256,718; 73,268,790; 73,286,900; 73,433,599; 73,444,913; 73,491,394; 73,540,570; 73,546,481; 73,581,205; 73,584,768; 73,820,614; 73,828,244; 73,847,393; 74,211,079; 74,312,211; 74,465,573; 74,522,550; 74,602,627; 74,698,144; 74,742,025; 74,744,031; 74,861,308; 74,888,146; 74,893,445; 74,938,563; 74,958,259; 74,965,847; 74,982,341; 75,138,633; 75,137,014; 75,141,986; 75,148,824; 75,161,143; 75,173,809; 75,179,788; 75,203,184; 75,226,884; 75,241,415; 75,253,891; 75,392,086; 75,480,618; 75,509,717; 75,545,324; 75,586,006; 75,591,421, 75,626,682; 75,800,407; 75,932,398; 76,104,437; 76,271,249; 76,430,984; 76,591,097; 76,793,466; 76,978,779; 77,232,337; 77,305,463; 77,449,286; 77,452,033; 77,567,942; 77,770,079; 77,858,300; 78,614,606; 78,887,311; 79,024,693; 79,263,154; 82,210,649; Abacus reference genome chromosome 2 position 85,807,792; Abacus reference genome chromosome 3 position 78,519,130; Abacus reference genome chromosome 4 position 65,565,100; Abacus reference genome chromosome 6 positions 4,712,978; 14,621,523; 20,187,255; 27,006,811; 49,434,383; Abacus reference genome chromosome 8 position 686,124; or Abacus reference genome chromosome 9 position 8,228,671 as described in Table 2.

In an embodiment, the marker comprises a polymorphism at a splice variant in the PRR37 Response Regulatory domain that results in a frameshift causing a premature stop codon. In an embodiment, the splice variant polymorphism is position 51 of SEQ ID NO:225. In an embodiment the polymorphism is a G to T polymorphism at position 26 of SEQ ID NO:225. In another embodiment, the markers comprise a truncated or deleted protein product of the endogenous PRR37 gene.

The present invention further describes the discovery of novel haplotype markers for plants, including Cannabis. Haplotypes refer to the genotype of a plant at a plurality of genetic loci, e.g., a combination of alleles or markers. Haplotype can refer to sequence polymorphisms at a particular locus, such as a single marker locus, or sequence polymorphisms at multiple loci along a chromosomal segment in a given genome. Markers present within the haplotype are significantly correlated to autoflowering plants, which thus can be used to screen plants exhibiting early autoflowering. In an embodiment, markers present within the haplotypes described in both Table 1 and Table 2 can be used to screen for autoflowering plants. Each of Table 1 and Table 2 describes the left and right flanking markers of the haplotype regions, as well as the left and right flanking marker position within the respective chromosome.

Accordingly, as a non-limiting example, Table 1 describes the marker identified as 132604_11137, which is located at position 65,423,973 on chromosome 1 of the Abacus Cannabis reference genome (or position 13 of SEQ ID NO:40), as a marker within a haplotype defined as being positioned between markers 166_371500 and 166_333448, or between positions 65,401,240 and 65,449,967 on chromosome 1 of the Abacus Cannabis reference genome. Thus any other marker that exists between positions 65,401,240 and 65,449,967 on chromosome 1 of the Abacus Cannabis reference genome is a marker imparting the autoflowering phenotype, which can be used to select for plants having autoflowering activity.

Similarly, Table 1 describes the marker identified as 166_325765, which is located at position 65,457,650 on chromosome 1 of the Abacus Cannabis reference genome (or position 13 of SEQ ID NO:41), as a marker within a haplotype defined as being positioned between markers 166_333488 and 166_297863, or between positions 65,449,967 and 65,485,211 on chromosome 1 of the Abacus Cannabis reference genome. Thus, any other marker that exists between positions 65,449,967 and 65,485,211 on chromosome 1 of the Abacus Cannabis reference genome is a marker imparting the autoflowering phenotype, which can be used to select for plants having autoflowering activity.

Thus, any marker existing within each haplotype described in Tables 1 or 2 is a marker imparting the autoflowering phenotype, which can be used to select for plants having autoflowering activity.

### Autoflowering Genes

In an embodiment, genes conferring an autoflowering phenotype are provided. The protein products of autoflowering genes UPF2 (SEQ ID NO:196), RAP2-7/TOE1 (SEQ ID NO:197), and PRR37 (SEQ ID NO:224) have been identified herein as being involved in the process of flowering in Cannabis based on the most significantly associated SNPs based on bulk segregant analysis (BSA). PRR37 has been shown in other plants to be a central repressor in the flowering regulatory pathway that controls flowering in response to day length. Sorghum plants containing a non-functional, truncated version of SbPRR37 caused by early termination before the Response Regulatory domain flower independently of photoperiod (=autoflowering phenotype), whereas sorghum plants containing the full length functional version of SbPRR37 flowered in response to photoperiod (=photosensitive phenotype; Murphy, R. L. et al. (2011), Proceedings of the National Academy of Sciences, 108(39), pp. 16469-16474).

Thus, the present invention provides one of skilled in the art the ability to edit a genome, as described herein, and subsequently select plants, having autoflowering activity based on the replacement of wild-type alleles with the allelic variants described herein conferring an autoflowering phenotype. The present invention further provides one of skill in the art the ability to edit a genome, as described herein, and subsequently select plants, having autoflowering activity based on the replacement of wild-type haplotypes with haplotypes known to be associated with autoflowering.

### Detection of Markers

Marker detection is well known in the art. For example, amplification of a target polynucleotide (e.g., by PCR) using a particular amplification primer pair that permit the primer pair to hybridize to the target polynucleotide to which a primer having the corresponding sequence (or its complement) would bind and preferably to produce an identifiable amplification product (the amplicon) having a marker is well known in the art.

Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It will be appreciated that suitable primers to be used with the invention can be designed using any suitable method. It is not intended that the invention be limited to any particular primer or primer pair. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. It is understood that a number of parameters in a specific PCR protocol may need to be adjusted to specific laboratory conditions and may be slightly modified and yet allow for the collection of similar results. The primers of the invention may be radiolabeled, or labeled by any suitable means (e.g., using a non-radioactive fluorescent tag), to allow for rapid visualization of the different size amplicons following an amplification reaction without any additional labeling step or visualization step. The known nucleic acid sequences for the genes described herein are sufficient to enable one of skill in the art to routinely select primers for amplification of the gene of interest.

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Aced. Sci. USA 86: 1173), self-sustained sequence replication (Guatelli et al. (1990) Proc. Natl. Aced. Sci. USA 87: 1874), dot PCR, and linker adapter PCR, etc.

An amplicon is an amplified nucleic acid, e.g., a nucleic acid that is produced by amplifying a template nucleic acid by any available amplification method (e.g., PCR, LCR, transcription, or the like). A genomic nucleic acid is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A template nucleic acid is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the invention be limited to any particular size.

Amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green 1 (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeders Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004. Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990. Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routedge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12(10):R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March, 51(2):371-5), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

In an embodiment, marker assisted selection (MAS) is used to produce plants with desired traits. MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with autoflowering may be assayed in progeny and those progeny with one or more desired markers are selected for advancement. In another aspect, one or more markers can be assayed in the progeny to select for plants with the genotype of the agronomically elite parent. This invention anticipates that trait introgressed autoflowering will require more than one generation, wherein progeny are crossed to the recurrent (agronomically elite) parent or selfed. Selections are made based on the presence of one or more autoflowering markers and can also be made based on the recurrent parent genotype, wherein screening is performed on a genetic marker and/or phenotype basis. In another embodiment, markers of this invention can be used in conjunction with other markers, ideally at least one on each chromosome of the Cannabis genome, to track the autoflowering phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present invention provides the means to identify plants that exhibit an autoflowering phenotype by identifying plants having autoflowering-specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant is not limited and can include any combination of markers disclosed herein or any marker linked to the markers disclosed herein, or any markers located within the QTL intervals defined herein.

In some embodiments, a first Cannabis plant or germplasm exhibiting a desired trait (the donor) can be crossed with a second Cannabis plant or germplasm (the recipient. e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene.

MAS, as described herein, using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein can thus find use within the scope of this invention.

Similarly, by identifying plants lacking a desired marker locus, plants lacking autoflowering activity, or plants having autoflowering activity, can be identified and eliminated from subsequent crosses. These marker loci can be introgressed into any desired genomic background, germplasm, plant, line, variety, etc., as part of an overall MAS breeding program designed to enhance autoflowering activity. The invention also provides chromosome QTL intervals that can be used in MAS to select plants that demonstrate different autoflowering traits. The QTL intervals can also be used to counter-select plants that do not exhibit autoflowering activity.

Thus, the invention permits one skilled in the art to detect the presence or absence of autoflowering genotypes in the genomes of Cannabis plants as part of a MAS program, as described herein. In one embodiment, a breeder ascertains the genotype at one or more markers for a parent having favorable autoflowering activity, which contains a favorable autoflowering activity allele, and the genotype at one or more markers for a parent with unfavorable autoflowering activity, which lacks the favorable autoflowering activity allele. A breeder can then reliably track the inheritance of the autoflowering activity alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having autoflowering activity alleles can be reliably predicted to express the desirable phenotype and progeny that share genotypes with the parent having unfavorable autoflowering activity alleles can be reliably predicted to express the undesirable phenotype. Thus, the laborious, inefficient, and potentially inaccurate process of manually phenotyping the progeny for autoflowering activity traits is avoided.

Closely linked markers flanking the locus of interest that have alleles in linkage disequilibrium with autoflowering activity alleles at that locus may be effectively used to select for progeny plants with desirable autoflowering activity traits. Thus, the markers described herein, such as those listed in Tables 1 and 2, as well as other markers genetically linked to the same chromosome interval, including but not limited to the marker at position 51 of SEQ ID NO:225, may be used to select for Cannabis plants with different autoflowering activity traits. Often, a haplotype, which is a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In an embodiment, MAS is used to select one or more Cannabis plants comprising autoflowering activity, the method comprising: (i) obtaining nucleic acids from the sample Cannabis plant or germplasm; (ii) detecting one or more markers that indicate autoflowering activity, (iii) indicating autoflowering activity, and (iv) selecting the one or more plants indicating autoflowering activity.

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype. Haplotypes of the present invention are described in the examples below, and can be used for marker assisted selection.

The choice of markers actually used to practice the invention is not limited and can be any marker that is genetically linked to the intervals as described herein, which includes markers mapping within the intervals. In certain embodiments, the invention further provides markers closely genetically linked to, or within approximately 0.5 cM of, the markers provided herein and chromosome intervals whose borders fall between or include such markers, and including markers within approximately 0.4 cM, 0.3 cM, 0.2 cM, and about 0.1 cM of the markers provided herein.

In some embodiments the markers and haplotypes described above can be used for marker assisted selection to produce additional progeny plants comprising the indicated autoflowering activity. In some embodiments, backcrossing may be used in conjunction with marker-assisted selection.

### Gene Editing

In some embodiments, gene editing is used to develop plants having autoflowering activity. In particular, methods for selecting one or more Cannabis plants having autoflowering activity, the method comprising replacing a nucleic acid sequence of a parent plant with a nucleic acid sequence conferring autoflowering activity. In some embodiments that method further comprises crossing or selfing the parent plant, thereby producing a plurality of progeny seed or clones, and selecting one or more progeny plants grown from the progeny seed or clone that comprise the nucleic acid sequence conferring autoflowering activity, thereby selecting modified autoflowering plants.

In an embodiment, a variant of UPF2 (having at least 90% sequence identity to SEQ ID NO:196) can be edited into a plant genome to confer autoflowering activity. The variant may have amino acid substitutions at one or more of amino acid positions 21, 23, 35, 40, 56, or 1230 relative to a wild-type amino acid sequence. The amino acid substitutions may include the amino acid substitutions identified herein.

In an embodiment, a variant of RAP2-7/TOE1 (having at least 90% sequence identity to SEQ ID NO:197) can be edited into a plant genome to confer autoflowering activity. The variant may have amino acid substitutions at one or more of amino acid positions 18 or 253, or a deletion between amino acids positions 35-37, relative to a wild-type amino acid sequence. The amino acid substitutions may include the amino acid substitutions identified herein.

In an embodiment, a transgenic plant whose genome comprises a homozygous deletion of an endogenous PRR37 gene is provided, which results in a Cannabis plant having autoflowering activity. The PRR37 transgenic plant comprises a genomic nucleic acid sequence having at least 90% sequence identity to SEQ ID NO:224 or a protein coding nucleic acid sequence having at least 90% sequence identity to SEQ ID NO:211. In some embodiments the homozygous deletion results in a truncated PPR37 amino acid sequence. In some embodiments, homozygous deletion comprises a protein coding nucleic acid sequence having at least 90% sequence identity to SEQ ID NOs:206, 207, 208, 209, or 210; or amino acid sequences having at least 90% sequence identity to SEQ ID NOs:213, 214, 215, 216, or 217.

Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In an embodiment, an isolated polynucleotide is provided comprising a nucleotide sequence having at least 72%, 73%, 74%, 75%, 76%, 77%, 78% 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST® can be used to compare specific regions of two sequences. A BLASTS comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST® match is considered herein as having an E-value for the top BLASTS hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST® alignment. Hit coverage refers to the percent of the database entry that is represented in the BLAST® alignment. In one embodiment of the invention, function of a query polypeptide is inferred from function of a protein homolog where either (1) hit_p<1e-30 or % identity>35% AND query_coverage>50% AND hit_coverage>50%, or (2) hit_p<1e-8 AND query_coverage>70% AND hit_coverage>70%. The following abbreviations are produced during a BLAST® analysis of a sequence. SEQ_NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBL_GI provides the GenBank ID number for the top BLAST® hit for the sequence. The top BLAST® hit is indicated by the National Center for Biotechnology Information GenBank Identifier number. NCBI_GI_DESCRIPTION refers to the description of the GenBank top BLAST® hit for sequence. E_VALUE provides the expectation value for the top BLAST® match. MATCH_LENGTH provides the length of the sequence which is aligned in the top BLAST® match TOP_HIT_PCT_IDENT refers to the percentage of identically matched nucleotides (or residues) that exist along the length of that portion of the sequences which is aligned in the top BLAST® match. CAT_TYPE indicates the classification scheme used to classify the sequence. GO_BP=Gene Ontology Consortium—biological process; GO_CC=Gene Ontology Consortium-cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLASTS hit which resulted in assignment of the sequence to the function category provided in the cat_desc column. HIT_E provides the E value for the BLASTS hit in the hit_desc column PCT_IDENT refers to the percentage of identically matched nucleotides (or residues) that exist along the length of that portion of the sequences which is aligned in the BLAST® match provided in hit_desc. CRY_RANGE lists the range of the query sequence aligned with the hit. HIT_RANGE lists the range of the hit sequence aligned with the query, provides the percent of query sequence length that matches CRY_CVRG provides the percent of query sequence length that matches to the hit (NCBI) sequence in the BLAST® match (% qry cvrg=(match length/query total length)×100). HIT_CVRG provides the percent of hit sequence length that matches to the query sequence in the match generated using BLASTS (% hit cvrg=(match lengthy hit total length)×100).

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, Calif.). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (.COPYRGT.1993-2016). DNASTAR. Madison, Wis.). The MegAlign program is a global sequence alignment program for polynucleotides or proteins.

Gene editing is well known in the art, and many methods can be used with the present invention. For example, a skilled artisan will recognize that the ability to engineer a trait relies on the action of the genome editing proteins and various endogenous DNA repair pathways. These pathways may be normally present in a cell or may be induced by the action of the genome editing protein. Using genetic and chemical tools to over-express or suppress one or more genes or elements of these pathways can improve the efficiency and/or outcome of the methods of the invention. For example, it can be useful to over-express certain homologous recombination pathway genes or suppression of non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from a native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), transcription activator-like effector nucleases (TALEN), any other mutagenesis procedure known to a person skilled in the art.

A skilled artisan would also appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system.

Methods for transformation of plant cells required for gene editing are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In planta transformation techniques (e.g., vacuum-infiltration, floral spraying or floral dip procedures) are well known in the art and may be used to introduce expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et at. 1998 Plant J. 16:735-743; and Desfeux et at. 2000 Plant Physiol 123:895-904). In these embodiments, seed produced by the plant comprise the expression cassettes encoding the genome editing proteins of the invention. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown and crossed with the same or different plant varieties using traditional breeding techniques to produce seed, which are then selected under the appropriate conditions.

The expression cassette can be integrated into the genome of the plant cells, in which case subsequent generations will express the genome editing proteins of the invention. Alternatively, the expression cassette is not integrated into the genome of the plants cell, in which case the genome editing proteins is transiently expressed in the transformed cells and is not expressed in subsequent generations.

A genome editing protein itself may be introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell. In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see Vergunst et al. 2000 Science 290:979-82).

### Kits for Use in Diagnostic Applications

Kits for use in diagnostic, research, and prognostic applications are also provided by the invention. Such kits may include any or all of the following: assay reagents, buffers, nucleic acids for detecting the target sequences and other hybridization probes and/or primers. The kits may include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), cloud-based media, and the like. Such media may include addresses to internet sites that provide such instructional materials.

### EXAMPLES

Aspects of the present teachings can be further understood in light of the following examples, which should not be construed as limiting the scope of the present teachings in any way.

The practice of the present teachings employ, unless otherwise indicated, conventional methods of protein chemistry biochemistry, recombinant DNA techniques and pharmacology, within the skill of the art. Such techniques are explained fully in the literature. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, Pa.; Carey and Sundberg, Advanced Organic Chemistry, Vols. A and B, 3rd Edition, 1992, Plenum Press.

The practice of the present teachings also employ, unless otherwise indicated, conventional methods of statistical analysis, within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons. Inc., NJ; T. Hastie et. al, The Elements of Statistical Learning: Data Mining, Inference, and Prediction, Second Edition 2009, Springer. N.Y.; W. Cooley and P. Lohnes, Multivariate procedures for the behavioral science 1962, John Wiley and Sons, Inc. NY; E. Jackson, A Users Guide to Principal Components 2003, John Wiley and Sons, Inc., NY.

### Example 1—Discovery of Cannabis Autoflower Markers

This example describes the discovery of the genetic basis for autoflowering. Two separate analyses were performed for the discovery of markers associated with the autoflowering phenotype. The first analysis made use of a set of three F2 populations derived from sib crosses between three genetically female F1 plants and one reversed female F1 pollen donor. The F1 plants were derived from a cross between readily available Cannabis varieties. Plants were grown in four blocks, 30 plants per block. In total 106 plants emerged and survived until phenotyping. Observations for flowering time and maturity were taken at a field site in Oregon during late August and early October 2019, respectively. In total, 20 plants were scored as “early” in August 2019 and were harvested late August/early September. The phenotype of these plants resembles the autoflower phenotype.

Array genotyping (Illumina bead array) was performed on 105 of the plants of the first analysis. This number included all 20 autoflower plants and 85 later flowering plants. All 105 plants were genotyped for 45,123 SNPs. Low quality and monomorphic SNPs were removed. Subsequently, SNPs with at least 90% of genotype data for the 105 accessions were kept for the bulk segregant analysis (n=30,810).

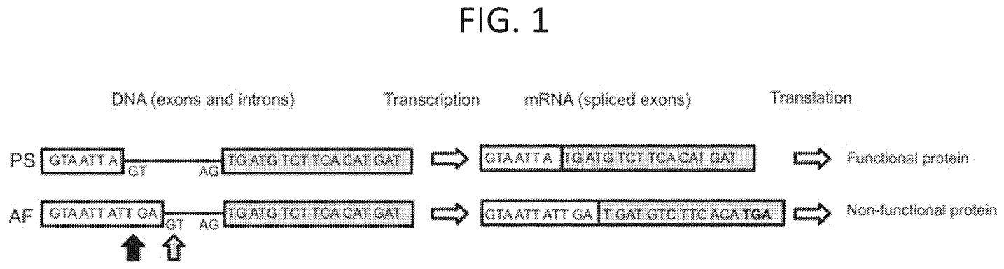

Two data sets were created for statistical analysis of the first analysis. The first data set contained for each SNP a count of the number of accessions that had the autoflower phenotype and were homozygous for the reference allele, as well as a count of the number of accessions that were later flowering and were homozygous for the reference allele. In addition, this data set contained for each SNP a count for accessions that were later flowering and that were either heterozygous or were homozygous for the alternative allele, as well as a count of the number of accessions that had the autoflower phenotype and that were either heterozygous or were homozygous for the alternative allele. The second data set contained for each SNP a count of the number of accessions that had the autoflower phenotype and were homozygous for the alternative allele, as well as a count of the number of accessions that were later flowering and were homozygous for the alternative allele. In addition, this data set contained for each SNP a count for accessions that were later flowering and that were either heterozygous or were homozygous for the reference allele, as well as a count of the number of accessions that had the autoflower phenotype and that were either heterozygous or were homozygous for the reference allele. Subsequently, these two data sets were analyzed using a Fisher exact test using the software R and SNPs were filtered for p-values smaller than 1.62E-06 (Bonferroni corrected p-value). The vast majority of the 214 significant SNP markers were located on chromosome 1, with only one SNP marker located on chromosome 9. It is expected that the associations observed for the marker located on chromosomes 9 was a false positive, which is likely even after multi-test Bonferroni correction due to the vast number of markers tested. The large number of markers in the same region on chromosome 1 showing a peak with increasing levels of significance provides a strong indication that this region is not a false positive, but that the strong association between phenotype and genotype is caused by a genetic factor located in this region on the Cannabis genome. A graph of the position in base pair by log 10 of the p-values shows a peak on chromosome 1 of the Abacus reference genome (version CsaAba2) (FIG. 1), and a graph of position in base pair by log 10 of the p-values within chromosome 1 shows the QTL peak at the end of chromosome 1 (FIG. 2).

Two markers displaying the most significant association (p=9.69E-22) with the autoflower phenotype in the first analysis were 166_325765 (position 65,457,650; SEQ ID NO:41)) and 132604_11137 (position 65,423,973; SEQ ID NO:40). Both markers displayed the homozygous alternative allele genotype for all 20 plants with the autoflower phenotype and displayed either a heterozygous or homozygous reference allele genotype for all 85 later flowering plants.

Marker 166_325765 is located inside the UPF2 gene and marker 132604_11137 located in the intergenic region, 33.7 Kb upstream from marker 166_325765. The nearest SNPs on either side of these two markers are marker 166_303719 (position 65,479,354) and marker 159_127948 (position 65,270,412). As a result, the haplotype associated with the autoflower phenotype is between 33.7-208.9 Kb long.

The second analysis involved one accession per seed lot of 12 autoflowering seed lots (Table 4) and one autoflowering accession from the F2 populations used in the first analysis, as well as a set of 63 photosensitive accessions. All plants were grown under 18 hours light in a greenhouse in Oregon and were checked weekly for flower development. The earliest flowering accessions started pre-flower (some pistils visible) during the 4th week after sow and were flowering during the 5th week after sow. Each accession was genotyped with an Illumina bead array. After initial marker QC, further filtering steps were performed to filter out known low quality SNPs, monomorphic SNPs, and SNPs with more than 5% of missing values. After these filtering steps, 34.916 array SNPs remained for analysis.

The BSA of the second analysis involved two Fisher Exact tests (using the software R) of a list of 4×4 tables, one row per SNP. The first test compared four categories: 1. Homozygous reference allele and autoflowering. 2. Homozygous reference allele and not autoflowering (photosensitive). 3. Heterozygous or homozygous alternate allele and autoflowering. 4. Heterozygous or homozygous alternate allele and not autoflowering (photosensitive). The second test compared four categories: 1. Homozygous alternate allele and autoflowering. 2. Homozygous alternate allele and not autoflowering (photosensitive). 3. Heterozygous or homozygous reference allele and autoflowering. 4. Heterozygous or homozygous alternate allele and not autoflowering (photosensitive). In total, 288 out of 34,916 SNP markers had significant p-values below the Bonferroni multi-test threshold of 1.43E-06. Seventy of these SNP markers overlapped with the set of 214 significantly associated SNP markers identified in the BSA based on the F2s. The vast majority of these SNP markers were located on chromosome 1 covering the same region as the vast majority of the significantly associated SNP markers identified in the F2s. Nine SNP markers were located dispersed across chromosomes 2, 3, 4, 6, and 8 (Table 1 and Table 2).

The two SNP markers (166_325765 and 132604_11137) that displayed the most significant association with the autoflower trait in the set of F2s were significantly associated with the autoflower trait in the second BSA (p=1.75E-09), however, SNPs in flanking regions were more significantly associated with the trait (p<1.75E-09; FIG. 3 and FIG. 4). A 6.3 Mbp region on chromosome 1 (Abacus reference genome version CsaAba2; positions 65,581,703-71,718,071 bp) flanking downstream of the two previously mapped SNP markers had the most significant association with the autoflowering phenotype. Significance levels varied based on the genotype of the photosensitive accessions. 14 SNP markers in this region were able to differentiate all 12 autoflower accessions (homozygous alternate) from all 63 photocenter accessions (homozygous reference or heterozygous; p=3.83E-14; Table 1). This flanking region was more significantly associated with the autoflowering trait because all accessions used in the analysis shared the flanking region genotype (homozygous alternate allele), but only a subset shared the homozygous alternate genotype for the two preferred SNP markers (166_325765 and 132604_11137; Table 1). A 4.0 Kbp region flanking upstream of the two preferred SNP markers contained two additional markers that had a similar level of significance as the two preferred SNP markers (p=1.75E-09; Table 1).

The autoflower QTL region includes 161 markers between position 63,161,656-71,980,891 on chromosome 1 of the Abacus reference genome that are associated based on high significance (p<1.0E-11) with the autoflower phenotype (Table 1). The full QTL region also includes a set of 10 markers between positions 65,129,138-75,648,136 on chromosome 1 that are associated based on lower significance with the autoflower phenotype but which are part of a set of multi-marker extended haplotypes that can discriminate between autoflowering and photosensitive accessions (Table 1). In addition, the full QTL region includes 255 markers between positions 268,476-82,210,649 on chromosome 1, one marker at position 85,807,792 on chromosome 2, one marker at position 78,519,130 on chromosome 3, one marker at position 85,565,100 on chromosome 4, five markers between positions 4,712,978-49,434,383 on chromosome 6, one marker at position 686,124 on chromosome 8, and one marker at position 8,228,671 on chromosome 9 that are significantly associated with the autoflower phenotype (below Bonferroni threshold; Table 2).

TABLE 1

| | | | | | | | | Position | Position |
|---|---|---|---|---|---|---|---|---|---|
| | | | | | Abacus | | | left | right |
| | | | | | reference | Left | Right | flanking | flanking |
| | | | | | genome | flanking | flanking | marker | marker |
| SNP marker | | | Ref | Alt | position | marker | marker | haplotype | haplotype |
| name | p-value | type | call | call | (bp) | haplotype | haplotype | (bp) | (bp) |
| 348_278501 | 1.67E−17 | B | T | C | 63,161,656 | 131142_22069 | 348_239922 | 63,130,064 | 63,197,706 |
| 136501_10493 | 6.88E−16 | B | C | A | 63,308,184 | 133296_15200 | 348_94453 | 63,307,519 | 63,329,004 |
| 348_68337 | 8.14E−20 | B | T | A | 63,355,114 | 348_72902 | Cannabis.v1_{—} | 63,350,549 | 63,361,058 |
| | | | | | | | scf2741- | | |
| | | | | | | | 29701_101 | | |
| 77102_2826 | 2.48E−15 | B | G | A | 63,422,002 | 348_43352 | 348_7429 | 63,374,097 | 63,437,224 |
| 78970_4740 | 8.14E−20 | B | A | G | 63,449,699 | 348_7429 | 109981_1829 | 63,437,224 | 63,475,188 |
| 130771_1619 | 3.89E−12 | A | C | G | 63,589,885 | 115082_8519 | 115082_11948 | 63,589,725 | 63,593,154 |
| 166_1420753 | 8.44E−16 | B | G | A | 63,675,478 | 166_1429525 | 166_1394159 | 63,661,793 | 63,714,225 |
| 166_1344599 | 8.14E−20 | B | T | C | 63,765,361 | 166_1394159 | 124419_1618 | 63,714,225 | 63,807,900 |
| 166_1342766 | 4.93E−14 | B | G | A | 63,767,236 | 166_1394159 | 124419_1618 | 63,714,225 | 63,807,900 |
| 70692_112 | 3.27E−15 | B | C | T | 63,775,211 | 166_1394159 | 124419_1618 | 63,714,225 | 63,807,900 |
| 137262_2355 | 1.21E−16 | B | T | A | 63,777,630 | 166_1394159 | 124419_1618 | 63,714,225 | 63,807,900 |
| 112864_918 | 1.21E−16 | B | T | A | 63,833,581 | 124419_8376 | 166_1267707 | 63,814,661 | 63,869,882 |
| 141828_41817 | 1.64E−13 | A | C | T | 63,925,984 | 141828_45835 | 141828_37657 | 63,921,962 | 63,930,131 |
| 137089_3738 | 3.89E−12 | A | G | A | 63,930,893 | 141828_37657 | 123680_447 | 63,930,131 | 63,941,822 |
| 141828_26051 | 3.89E−12 | A | G | A | 63,945,679 | 123680_447 | 141828_22202 | 63,941,822 | 63,948,723 |
| 166_1216813 | 1.45E−12 | A | A | T | 64,035,782 | 166_1240650 | 190862_1647 | 64,014,612 | 64,068,363 |
| 166_1210832 | 4.93E−14 | B | C | T | 64,041,749 | 166_1240650 | 190862_1647 | 64,014,612 | 64,068,363 |
| 166_1072196 | 1.21E−16 | B | C | T | 64,187,259 | 166_1077758 | 166_1066073 | 64,179,404 | 64,196,042 |
| 166_1050755 | 1.67E−17 | B | G | A | 64,233,047 | 166_1063096 | 166_1035743 | 64,199,156 | 64,245,883 |
| 166_1042556 | 8.14E−20 | B | A | G | 64,238,617 | 166_1063096 | 166_1035743 | 64,199,156 | 64,245,883 |
| 166_1026787 | 8.14E−20 | B | A | T | 64,253,959 | 166_1035743 | 132274_7993 | 64,245,883 | 64,259,638 |
| 79036_402 | 1.34E−14 | B | A | G | 64,254,725 | 166_1035743 | 132274_7993 | 64,245,883 | 64,259,638 |
| 104702_6585 | 3.89E−12 | A | T | A | 64,261,547 | 132274_7993 | 166_990096 | 64,259,638 | 64,331,404 |
| 104702_4384 | 1.45E−12 | A | A | G | 64,262,905 | 132274_7993 | 166_990096 | 64,259,638 | 64,331,404 |
| 166_976188 | 1.45E−12 | A | G | C | 64,349,232 | 166_982294 | 141264_25818 | 64,341,256 | 64,354,653 |
| 141264_16477 | 8.14E−20 | B | G | T | 64,363,968 | 141264_25818 | 126819_11268 | 64,354,653 | 64,369,895 |
| 126819_3234 | 8.14E−20 | B | G | A | 64,377,929 | 126819_11268 | 131502_16928 | 64,369,895 | 64,430,126 |
| 166_800955 | 5.05E−13 | B | C | T | 64,515,399 | 166_806951 | Cannabis.v1_{—} | 64,510,032 | 64,516,179 |
| | | | | | | | cf4196- | | |
| | | | | | | | 10473_101 | | |
| 126791_897 | 1.64E−13 | B | T | C | 64,575,147 | 77772_965 | 166_695015 | 64,573,181 | 64,653,136 |
| 166_684489 | 7.31E−20 | B | C | G | 64,663,448 | 166_691509 | 166_637730 | 64,656,486 | 64,699,756 |
| 101156_900 | 3.27E−15 | B | C | T | 64,686,430 | 166_691509 | 166_637730 | 64,656,486 | 64,699,756 |
| 166_514787 | 1.67E−17 | B | G | A | 64,879,585 | 166_558884 | 166_507449 | 64,796,283 | 64,886,899 |
| 118257_5997 | 1.67E−17 | B | G | C | 64,920,471 | 102988_1305 | 166_469005 | 64,919,176 | 64,940,198 |
| 166_408348 | 8.14E−20 | B | G | A | 65,004,163 | 166_418045 | Cannabis.v1_{—} | 64,994,469 | 65,022,221 |
| | | | | | | | scf1198- | | |
| | | | | | | | 99332_100 | | |
| 130617_9054** | 8.14E−20 | B | C | T | 65,022,166 | 166_418045 | Cannabis.v1_{—} | 64,994,469 | 65,022,221 |
| | | | | | | | scf1198- | | |
| | | | | | | | 99332_100 | | |
| 159_49348 | 1.45E−12 | A | T | C | 65,181,429 | 159_24455 | 139534_1583 | 65,156,550 | 65,184,720 |
| 138054_8795** | 8.14E−20 | B | G | T | 65,183,123 | 159_24455 | 139534_1583 | 65,156,550 | 65,184,720 |
| 159_79356 | 2.04E−20 | A | T | C | 65,220,358 | 159_74552 | 159_84541 | 65,215,554 | 65,225,655 |
| 159_127948 | 9.71E−15 | A | A | G | 65,270,412 | 159_119575 | 159_131820 | 65,260,467 | 65,275,225 |
| 132604_11137* | 9.69E−22 | B | T | C | 65,423,973 | 166_371500 | 166_333448 | 65,401,240 | 65,449,967 |
| 166_325765* | 9.69E−22 | B | A | G | 65,457,650 | 166_333448 | 166_297863 | 65,449,967 | 65,485,211 |
| 166_303719** | 2.24E−19 | B | T | G | 65,479,355 | 166_333448 | 166_297863 | 65,449,967 | 65,485,211 |
| 166_273040 | 2.04E−20 | B | G | T | 65,510,077 | 166_288842 | 109117_1157 | 65,494,231 | 65,572,130 |
| 166_250111 | 2.23E−16 | B | C | T | 65,533,197 | 166_288842 | 109117_1157 | 65,494,231 | 65,572,130 |
| 340_14470** | 8.14E−20 | B | C | T | 65,581,703 | 340_7955 | 171318_702 | 65,575,227 | 65,594,497 |
| 120836_5326 | 1.21E−16 | B | A | C | 65,586,925 | 340_7955 | 171318_702 | 65,575,227 | 65,594,497 |
| 130163_5010 | 8.14E−20 | B | A | G | 66,123,957 | 163919_464 | 167_836411 | 66,100,344 | 66,328,377 |
| 275_711038 | 8.14E−20 | B | T | C | 66,213,077 | 163919_464 | 167_836411 | 66,100,344 | 66,328,377 |
| 275_448497 | 1.67E−17 | B | C | T | 66,540,589 | 275_448863 | 275_425588 | 66,540,216 | 66,571,454 |
| 132275_2738 | 5.05E−13 | A | T | C | 66,925,020 | 75565_5431 | 142427_18192 | 66,899,701 | 66,936,328 |
| 122921_3232 | 8.14E−20 | B | G | A | 67,609,581 | 238_1215 | 238_29539 | 67,602,284 | 67,635,242 |
| 211_215197 | 1.67E−17 | B | C | T | 67,695,735 | 211_217708 | 211_206218 | 67,691,271 | 67,704,743 |
| 211_201852 | 5.05E−13 | B | T | C | 67,708,527 | 211_206218 | 134508_8071 | 67,704,743 | 67,720,656 |
| 211_198786 | 3.27E−15 | B | C | T | 67,711,595 | 211_206218 | 134508_8071 | 67,704,743 | 67,720,656 |
| 177642_4242 | 3.27E−15 | B | G | A | 67,761,686 | 177642_6779 | 211_142022 | 67,754,189 | 67,765,486 |
| 211_126528 | 8.14E−20 | B | T | G | 67,780,949 | 211_142022 | 211_72686 | 67,765,486 | 67,846,474 |
| 211_60979 | 6.88E−16 | B | G | C | 67,858,135 | 211_70408 | 211_53920 | 67,848,778 | 67,871,696 |
| 211_40813 | 5.05E−13 | A | G | T | 67,892,254 | 211_53920 | 211_24731 | 67,871,696 | 67,909,056 |
| 211_14204 | 3.27E−15 | B | A | T | 67,919,111 | 211_24731 | 107871_2783 | 67,909,056 | 67,933,738 |
| 79134_4041 | 1.64E−13 | B | T | C | 67,972,467 | 107871_2783 | 139467_64516 | 67,933,738 | 68,018,692 |
| 300_332172 | 1.61E−18 | B | A | G | 68,100,304 | 102856_300 | 300_317551 | 68,099,919 | 68,121,467 |
| 141037_9199 | 1.21E−16 | B | G | A | 68,184,751 | 300_317551 | 300_204627 | 68,121,467 | 68,231,239 |
| 157129_5206 | 8.14E−20 | B | G | A | 68,393,736 | 300_151632 | 112293_8428 | 68,312,784 | 68,417,685 |
| 142410_24821 | 8.14E−20 | B | G | A | 68,451,268 | 142410_19124 | 142410_32873 | 68,445,440 | 68,462,870 |
| 141410_24865 | 3.46E−18 | B | C | T | 69,116,895 | 139190_36604 | 134764_14714 | 69,107,887 | 69,226,040 |
| 135301_3997 | 2.31E−14 | B | G | A | 69,243,942 | 134784_3655 | 182238_3757 | 69,236,985 | 69,258,864 |
| 125861_674 | 6.28E−16 | B | A | G | 69,255,336 | 134764_3655 | 182238_3757 | 69,236,985 | 69,258,864 |
| 182237_22983 | 1.41E−12 | B | G | C | 69,275,241 | 182235_23731 | Cannabis.v1_{—} | 69,274,518 | 69,278,729 |
| | | | | | | | cf585- | | |
| | | | | | | | 250_100 | | |
| 91363_1648 | 4.30E−15 | B | T | C | 69,304,025 | Cannabis.v1_{—} | 182237_901 | 69,278,729 | 69,308,712 |
| | | | | | | scf585- | | | |
| | | | | | | 250_100 | | | |
| 171614_7078 | 3.46E−18 | B | T | A | 69,469,022 | 171616_4690 | 171614_1778 | 69,452,508 | 69,474,322 |
| 139100_11454 | 1.04E−13 | B | G | A | 70,249,642 | 145869_560 | 142164_2682 | 69,750,182 | 70,364,874 |
| 165_38630 | 6.43E−17 | B | G | A | 70,580,989 | 165_10311 | 165_70162 | 70,552,676 | 70,622,928 |
| 165_43011 | 4.30E−15 | B | C | T | 70,585,368 | 165_10311 | 165_70162 | 70,552,676 | 70,622,928 |
| 100294_1649 | 6.43E−17 | B | G | A | 70,587,829 | 165_10311 | 165_70162 | 70,552,676 | 70,622,928 |
| 121703_4932 | 6.43E−17 | B | G | A | 70,614,319 | 165_10311 | 165_70162 | 70,552,676 | 70,622,928 |
| 165_61499 | 1.04E−13 | B | T | C | 70,614,532 | 165_10311 | 165_70162 | 70,552,676 | 70,622,928 |
| 165_76110 | 7.79E−17 | B | T | G | 70,624,359 | 165_70162 | 165_78825 | 70,622,928 | 70,654,084 |
| 121858_4181 | 6.43E−17 | B | A | G | 70,686,503 | 165_121871 | 165_168932 | 70,677,515 | 70,696,509 |
| 228_36608 | 4.05E−13 | B | C | G | 70,884,481 | 228_39438 | 228_25726 | 70,881,651 | 70,903,223 |
| 165_523583 | 2.07E−15 | B | T | C | 71,067,519 | 228_17995 | 165_532124 | 70,911,075 | 71,076,061 |
| 165_527008 | 1.73E−15 | B | C | T | 71,070,939 | 228_17995 | 165_532124 | 70,911,075 | 71,076,061 |
| 133827_10943 | 1.73E−15 | B | C | T | 71,359,028 | 139564_29461 | 139808_29916 | 71,271,690 | 71,359,842 |
| 244_4611 | 1.73E−15 | B | T | C | 71,550,096 | 244_20150 | 107508_5859 | 71,534,556 | 71,585,672 |
| 140627_3175 | 1.73E−15 | B | C | G | 71,671,694 | 107508_5859 | 140627_26569 | 71,585,672 | 71,707,590 |
| 113372_17427 | 5.18E−13 | B | C | A | 71,695,399 | 107508_5859 | 140627_26569 | 71,585,672 | 71,707,590 |
| 100680_1353 | 1.73E−15 | B | G | A | 71,718,071 | 140627_26569 | 140627_39787 | 71,707,590 | 71,727,529 |
| 122185_1743 | 1.22E−13 | B | T | C | 71,824,879 | 140627_39787 | 141926_2591 | 71,727,529 | 71,837,480 |
| 138896_55595 | 1.02E−13 | A | C | T | 71,858,474 | 141926_2591 | 141186_17297 | 71,837,480 | 71,951,074 |
| 140727_152015 | 1.02E−13 | B | T | C | 72,378,842 | 79185_3265 | 140727_135845 | 72,376,736 | 72,395,693 |
| 190653_3284 | 1.73E−15 | B | A | G | 72,454,019 | 140727_128382 | 190653_1231 | 72,403,156 | 72,456,095 |
| 190652_1885 | 8.14E−12 | B | T | A | 72,455,436 | 140727_12832 | 190653_1231 | 72,403,156 | 72,456,095 |
| 131552_1374 | 1.73E−15 | B | G | A | 72,743,748 | 179171_1728 | 179170_45435 | 72,721,249 | 72,798,781 |
| 121_580459 | 1.83E−12 | B | T | A | 73,473,406 | 121_531793 | 131166_13901 | 73,366,820 | 73,483,248 |
| 121_626183 | 1.83E−12 | B | A | G | 73,517,405 | 121_618608 | 121_634632 | 73,509,920 | 73,525,854 |
| 141849_964 | 1.83E−12 | B | A | G | 73,817,673 | 141178_2275 | 129816_2540 | 73,799,827 | 73,840,855 |
| 141849_8876 | 1.83E−12 | B | G | A | 73,826,184 | 141178_2275 | 129816_2540 | 73,799,827 | 73,840,855 |
| 129816_7004 | 1.83E−12 | B | G | T | 73,836,391 | 141178_2275 | 129816_2540 | 73,799,827 | 73,840,855 |
| 141048_10203 | 1.02E−13 | B | T | C | 73,911,833 | 141048_3173 | 120318_111 | 73,896,283 | 73,936,788 |
| 204_79850 | 2.20E−12 | B | T | C | 73,982,309 | 204_86059 | 204_71202 | 73,972,289 | 73,990,969 |
| 141677_8625 | 1.83E−12 | B | A | G | 74,787,289 | 141677_13830 | 137322_15866 | 74,781,979 | 74,900,777 |
| 91362_6436 | 7.10E−13 | B | C | T | 77,758,271 | 127428_12466 | 127738_1041 | 77,734,932 | 77,767,257 |
| 292_136433 | 3.50E−12 | B | C | A | 78,122,009 | 292_148465 | 123790_672 | 78,097,597 | 78,140,005 |
| 300_84463 | 8.14E−20 | B | C | A | 48,727,601 | 300_56425 | 140347_5675 | 48,670,446 | 48,757,509 |
| 348_160959 | 3.48E−12 | B | A | T | 63,267,403 | 348_163506 | 348_150758 | 63,260,374 | 63,277,604 |
| 348_157790 | 3.48E−12 | B | A | G | 63,270,572 | 348_163506 | 348_150758 | 63,260,374 | 63,277,604 |
| 100933_1600 | 2.17E−12 | A | G | A | 63,358,922 | 348_72902 | Cannabis.v1_{—} | 63,350,549 | 63,361,058 |
| | | | | | | | scf2741- | | |
| | | | | | | | 29701_101 | | |
| 348_4479 | 1.39E−13 | A | G | A | 63,445,606 | 348_7429 | 109981_1829 | 63,437,224 | 63,475,188 |
| 137952_1704 | 4.95E−12 | B | C | T | 63,542,841 | 63475_816 | 139099_713 | 63,483,879 | 63,560,751 |
| 133211_9562 | 3.04E−13 | A | G | T | 63,622,828 | 109648_2740 | 111489_1133 | 63,599,571 | 63,638,953 |
| Cannabis.v1_{—} | 3.89E−12 | A | A | T | 63,721,208 | 166_1394159 | 137262_256 | 63,714,225 | 63,775,531 |
| scf1886- | | | | | | | | | |
| 705_100 | | | | | | | | | |
| Cannabis.v1_{—} | 1.64E−13 | A | C | A | 63,723,647 | 166_1429525 | 137262_256 | 63,661,793 | 63,775,531 |
| scf1886- | | | | | | | | | |
| 3142_101 | | | | | | | | | |
| 113863_2182 | 3.48E−12 | B | G | T | 64,003,743 | 166_1255129 | 113863_990 | 64,002,726 | 64,004,936 |
| Cannabis.v1_{—} | 5.05E−13 | A | G | C | 64,037,854 | 166_1240650 | 190862_1647 | 64,014,612 | 64,068,363 |
| scf3513- | | | | | | | | | |
| 33786_101 | | | | | | | | | |
| 130617_11900 | 3.89E−12 | A | C | T | 65,019,322 | 166_418045 | Cannabis.v1_{—} | 64,994,469 | 65,022,221 |
| | | | | | | | scf1198- | | |
| | | | | | | | 99332_100 | | |
| 159_2273 | 3.89E−12 | A | G | A | 65,050,650 | 125670_6114 | 159_8752 | 65,046,021 | 65,129,138 |
| 159_17477 | 3.89E−12 | A | G | A | 65,137,864 | 159_8752 | 159_24455 | 65,129,138 | 65,156,550 |
| 159_41757 | 1.45E−12 | A | G | T | 65,173,837 | 159_24455 | 139534_1583 | 65,156,550 | 65,184,720 |
| 138054_6707 | 1.45E−12 | A | T | C | 65,181,426 | 159_24455 | 139534_1583 | 65,156,550 | 65,184,720 |
| 275_764127 | 1.67E−17 | B | A | G | 65,761,925 | 275_777432 | 275_729971 | 65,748,622 | 65,803,381 |
| 275_642249 | 1.67E−17 | B | A | G | 65,886,304 | 275_654722 | 275_621026 | 65,870,981 | 65,907,684 |
| 275_605120 | 1.67E−17 | A | T | C | 65,927,579 | 275_721798 | 163919_464 | 65,808,147 | 66,100,344 |
| 275_599093 | 8.14E−20 | B | G | A | 65,933,598 | 275_614389 | 167_836411 | 65,914,321 | 66,328,377 |
| 275_581421 | 1.45E−12 | A | G | A | 65,963,869 | 167_836411 | 275_576778 | 66,328,377 | 66,378,083 |
| 141029_14247 | 3.48E−12 | B | C | T | 65,985,313 | 141029_18165 | 141029_9389 | 65,983,001 | 65,990,175 |
| 141029_9389 | 3.74E−15 | B | C | A | 65,990,175 | 141029_14247 | 141029_3963 | 65,985,314 | 65,995,578 |
| 79111_346 | 1.45E−12 | A | G | A | 66,001,667 | 275_721798 | 163919_464 | 65,808,147 | 66,100,344 |
| 105272_527 | 1.21E−16 | B | T | C | 66,015,507 | 275_721798 | 163919_464 | 65,808,147 | 66,100,344 |
| 165719_1761 | 8.14E−20 | B | T | C | 66,099,050 | 275_491530 | 275_482387 | 66,071,117 | 66,511,409 |
| 275_458039 | 3.89E−12 | A | C | T | 66,531,090 | 275_482387 | 275_448863 | 66,511,409 | 66,540,216 |
| 275_302105 | 5.05E−13 | B | C | T | 66,665,268 | 275_322068 | 107931_9009 | 66,645,983 | 66,671,851 |
| 139438_4208 | 8.14E−20 | A | G | A | 66,683,626 | 139438_8370 | 275_142359 | 67,067,970 | 67,113,644 |
| 275_117110 | 8.14E−20 | B | A | C | 66,740,867 | 275_142359 | 275_215992 | 67,113,644 | 67,173,457 |
| 275_33664 | 5.05E−13 | A | T | C | 66,834,787 | 275_40266 | 103507_4981 | 66,828,176 | 86,837,491 |
| 141279_11648 | 3.48E−12 | B | T | C | 66,983,293 | 142427_49217 | 141279_9178 | 66,969,241 | 66,985,755 |
| 275_309233 | 4.93E−14 | A | C | T | 67,034,241 | 275_322068 | 107931_9009 | 66,645,983 | 66,671,851 |
| 275_126666 | 4.93E−14 | A | G | T | 67,129,334 | 275_142359 | 275_215992 | 67,113,644 | 67,173,457 |
| 139608_9837 | 3.48E−12 | B | C | T | 67,454,121 | 175555_1001 | 90640_1086 | 67,450,523 | 67,491,728 |
| 140521_15210 | 4.15E−12 | B | C | A | 67,498,547 | 79075_2893 | 140521_16518 | 67,495,861 | 67,499,864 |
| 140258_1471 | 4.15E−12 | B | T | G | 67,585,755 | 134455_8389 | 118250_1035 | 67,566,014 | 67,588,554 |
| 238_1215 | 3.48E−12 | B | G | T | 67,602,283 | 118250_1035 | 238_24099 | 67,588,554 | 67,629,801 |
| 238_24099 | 1.45E−12 | A | G | C | 67,629,801 | 238_1215 | 238_29539 | 67,602,284 | 67,635,242 |
| 211_30316 | 1.45E−12 | A | A | T | 67,903,472 | 211_53920 | 211_24731 | 67,871,696 | 67,909,056 |
| 139467_31539 | 1.45E−12 | A | T | C | 67,976,538 | 107871_2783 | 139467_64516 | 67,933,738 | 68,018,692 |
| 142410_20135 | 1.45E−12 | A | G | A | 68,446,452 | 142410_19124 | 142410_32873 | 68,445,440 | 68,462,870 |
| 300_117302 | 1.45E−12 | A | A | C | 68,470,691 | 142410_32873 | 300_80735 | 68,462,870 | 68,520,102 |
| 102355_181 | 6.19E−13 | A | G | T | 68,493,804 | 142410_32873 | 300_80735 | 68,462,870 | 68,520,102 |
| 300_32251 | 8.14E−20 | B | C | G | 68,567,745 | 300_25363 | 165_7042 | 68,574,567 | 68,922,098 |
| 181985_1530 | 6.43E−17 | B | G | A | 68,887,689 | 138421_15228 | 165_10311 | 68,740,664 | 70,552,676 |
| 181984_9517 | 6.43E−17 | B | G | A | 68,899,476 | 138421_15228 | 165_10311 | 68,740,664 | 70,552,676 |
| 165_18641 | 6.43E−17 | B | G | A | 68,932,932 | 165_10311 | 165_70162 | 70,552,676 | 70,622,928 |
| 101368_913 | 2.31E−14 | A | A | G | 69,078,399 | 139190_36604 | 145869_560 | 69,107,887 | 69,750,182 |
| 120315_3015 | 2.8E−14 | B | G | A | 69,415,301 | 138694_2562 | 171614_1778 | 69,367,457 | 69,474,322 |
| 171616_8850 | 3.46E−18 | B | C | A | 69,448,252 | 171614_27758 | 136300_79178 | 69,448,348 | 70,520,852 |
| 171614_23426 | 3.46E−18 | B | C | T | 69,452,673 | 138694_2562 | 171614_1778 | 69,367,457 | 69,474,322 |
| 138744_7043 | 4.48E−12 | A | G | C | 69,496,492 | 145861_3311 | 171614_1778 | 69,061,049 | 69,474,322 |
| 142164_25210 | 2.31E−14 | A | T | C | 69,561,200 | 142164_20305 | 142384_15710 | 69,556,342 | 69,580,641 |
| 142384_11837 | 2.31E−14 | B | C | A | 69,576,766 | 142164_20305 | 142384_15710 | 69,556,342 | 69,580,641 |
| 139190_27380 | 6.28E−16 | B | T | G | 69,803,046 | 139190_36604 | 141509_709741 | 69,107,887 | 70,543,494 |
| 142164_495 | 3.46E−18 | B | C | T | 70,367,062 | 171616_4690 | 142164_2682 | 69,452,508 | 70,364,874 |
| 115119_7254 | 4.15E−12 | B | A | T | 71,980,891 | 100633_619 | 102579_950 | 71,976,623 | 71,989,503 |
| 221_24913** | 1.75E−09 | B | G | T | 75,648,136 | 221_8196 | 221_30094 | 75,630,089 | 75,655,379 |
| 121_398155 | 1.83E−12 | A | T | C | 74,962,881 | 131285_3785 | 121_385289 | 74,956,520 | 74,976,464 |
| 159_74552** | 3.52E−08 | A | G | A | 65,215,553 | 159_65486 | 159_79356 | 65,197,566 | 65,220,357 |
| 275_654722** | 3.70E−07 | B | C | A | 65,870,980 | 132506_1735 | 275_621026 | 65,861,415 | 65,907,683 |
| 275_564391** | 4.82E−09 | B | A | T | 6,5980,912 | 275_567160 | 141029_18165 | 6,5978,145 | 65,983,001 |
| 159_8752** | 3.41E−08 | B | A | G | 65,129,138 | 159_2273 | 159_17477 | 65050651 | 65137865 |
| 159_103549** | 3.06E−02 | B | G | C | 65,244,439 | NA | NA | NA | NA |
| 122130_2019** | 8.29E−02 | B | A | T | 65,470,698 | NA | NA | NA | NA |
| 166_297863** | 3.07E−03 | B | C | T | 65,485,211 | NA | NA | NA | NA |
| 109117_1157** | 5.88E−02 | B | A | G | 65,572,130 | NA | NA | NA | NA |
| 171326_1256** | 1.33E−09 | B | A | T | 65,601,780 | 171318_702 | 171327_3432 | 65594497 | 65611129 |

Significantly associated SNP markers with the autoflower phenotype identified in the two BSA analyses (p-values < 1.00E−11) as well as SNP markers which are part of the set of haplotypes which can identify autoflowering accessions (marked with **).
First column, SNP marker name; Second column, p-value; Third column, genotype associated with the autoflowering phenotype (A = homozygous for reference allele, B = homozygous for alternative allele); Fourth column, reference allele call; Fifth column, alternative allele call, Sixth column, Abacus reference genome position; Seventh column left flanking SNP of haplotype; Eight column, right flanking SNP of haplotype; Ninth column, Abacus reference genome position of left flanking SNP of haplotype; Tenth column, Abacus reference genome position of right flanking SNP of haplotype.
*In certain autoflowering genetic backgrounds these SNP markers can have genotype A or X, in these backgrounds a set of extended haplotypes consisting of these two SNP markers and 10 additional SNP markers (marked with **; p-values from BSA comparing 12 autoflowering accessions with 63 photosensitive accessions) is associated with autoflower (as shown in Table 5).
Haplotypes could not be located for four of these SNP markers and were marked with “NA”.

TABLE 2

| | | | | | | | | Position | Position |
|---|---|---|---|---|---|---|---|---|---|
| | | | | | Abacus | | | left | right |
| | | | | | reference | Left | Right | flanking | flanking |
| | | | | | genome | flanking | flanking | marker | marker |
| SNP marker | | | Ref | Alt | position | marker | marker | haplotype | haplotype |
| name | p-value | type | call | call | (bp) | haplotype | haplotype | (bp) | (bp) |
| 134481_27967 | 8.85687E−08 | X | T | C | 268,476 | 90_137130 | 134481_25995 | 240,794 | 270,448 |
| 90_2230480 | 1.05601E−06 | A | A | G | 3,326,542 | 90_2226361 | 90_2247969 | 3,322,994 | 3,344,675 |
| 130637_273 | 1.05601E−06 | B | A | G | 15,402,934 | 131417_380 | 123585_15303 | 15,377,463 | 15,408,489 |
| 157_3566147 | 1.25829E−06 | B | G | C | 16,672,487 | 157_3562001 | 157_3578172 | 16,668,341 | 16,684,512 |
| 365_1221706 | 2.06618E−07 | B | G | A | 19,090,442 | 139521_3606 | 365_1207063 | 19,076,801 | 19,105,086 |
| 369_93687 | 5.34423E−07 | B | C | G | 20,962,173 | 369_72081 | 369_98125 | 20,940,570 | 20,966,611 |
| 192_100941 | 4.23094E−10 | A | G | C | 25,416,995 | 137931_4324 | 192_7560 | 25,294,579 | 25,525,937 |
| 141684_19962 | 4.72044E−07 | B | A | C | 25,975,749 | 139055_47254 | 141684_13624 | 25,942,087 | 25,982,087 |
| 75376_815 | 1.05601E−06 | A | G | A | 27,376,279 | 161_1804450 | 161_1813952 | 27,369,634 | 27,379,064 |
| 161_1636900 | 1.05601E−06 | A | T | C | 27,463,437 | 161_1642365 | 161_1634304 | 27,457,973 | 27,466,037 |
| 161_1571007 | 1.25829E−06 | A | G | C | 27,527,476 | 161_1574876 | 161_1558496 | 27,522,060 | 27,539,985 |
| Cannabis.v1_{—} | 1.92881E−09 | B | A | G | 30,742,977 | 139082_1467 | 161_297069 | 30,706,746 | 30,752,016 |
| scf3455- | | | | | | | | | |
| 23197_100 | | | | | | | | | |
| 161_200382 | 2.36871E−10 | B | G | A | 30,874,960 | 141667_26262 | 161_159195 | 30,846,285 | 30,903,727 |
| 161_192384 | 8.45969E−10 | B | G | T | 30,883,438 | 141667_26262 | 161_159195 | 30,846,285 | 30,903,727 |
| 161_163657 | 3.69688E−07 | B | G | A | 30,899,325 | 141667_26262 | 161_159195 | 30,846,285 | 30,903,727 |
| 161_55526 | 2.36871E−10 | B | T | A | 31,017,608 | 161_65693 | 113451_3607 | 31,013,144 | 31,019,992 |
| 75238_628 | 1.03513E−07 | B | A | T | 31,082,669 | 133020_1322 | 141407_5644 | 31,040,629 | 31,088,901 |
| 131056_5316 | 4.82202E−09 | B | A | G | 31,164,922 | 131056_636 | 137305_430 | 31,160,242 | 31,172,432 |
| 271_323450 | 3.69688E−07 | B | T | C | 32,317,496 | 128706_6737 | 271_305729 | 32,301,016 | 32,361,583 |
| 142423_3172 | 7.10614E−10 | B | G | A | 32,459,479 | 142423_8107 | 271_175440 | 32,454,530 | 32,488,607 |
| 276_91753 | 1.92881E−09 | A | T | G | 32,941,839 | 276_99309 | Cannabis.v1_{—} | 32,933,101 | 32,955,189 |
| | | | | | | | scf3606- | | |
| | | | | | | | 12159_100 | | |
| 143921_14077 | 8.60071E−08 | B | C | A | 33,407,180 | 127020_253 | 141915_1321 | 33,398,552 | 33,423,460 |
| 349_556628 | 1.03513E−07 | A | C | A | 33,692,404 | 124258_977 | 126017_557 | 33,677,174 | 33,702,932 |
| 349_470195 | 3.69688E−07 | A | C | T | 33,809,865 | 349_475098 | 349_467518 | 33,804,986 | 33,812,537 |
| 138124_14336 | 1.92881E−09 | A | A | T | 33,867,472 | 82590_9012 | 138124_1389 | 33,852,994 | 33,876,863 |
| Cannabis.v1_{—} | 4.82202E−09 | A | A | G | 33,882,304 | 138124_1389 | 349_380352 | 33,876,863 | 33,888,237 |
| scf8324- | | | | | | | | | |
| 861_100 | | | | | | | | | |
| 349_350197 | 5.7405E−09 | A | C | T | 33,915,586 | 129508_2160 | 349_344694 | 33,901,818 | 33,923,705 |
| 349_200862 | 3.36568E−10 | A | C | A | 34,104,715 | 349_212440 | Cannabis.v1_{—} | 34,092,969 | 34,105,420 |
| | | | | | | | scf4133- | | |
| | | | | | | | 34499_101 | | |
| 349_194241 | 2.36871E−10 | A | G | A | 34,111,342 | Cannabis.v1_{—} | 349_187642 | 34,105,420 | 34,124,256 |
| | | | | | | scf4133- | | | |
| | | | | | | 34499_101 | | | |
| 349_92589 | 1.16452E−06 | A | A | G | 34,236,079 | 137894_8117 | 349_90556 | 34,211,259 | 34,238,101 |
| 349_7045 | 2.36871E−10 | B | C | G | 34,335,660 | 349_11434 | 118986_15006 | 34,331,250 | 34,341,741 |
| 139105_14416 | 1.03513E−07 | B | T | A | 34,390,673 | 103069_1860 | 163337_1186 | 34,374,895 | 34,393,567 |
| 139105_10156 | 1.99063E−07 | B | A | G | 34,403,630 | 163337_1186 | 91289_4379 | 34,393,567 | 34,431,603 |
| 138925_2792 | 3.69688E−07 | B | C | G | 34,443,652 | 120898_395 | 111797_19942 | 34,440,524 | 34,446,979 |
| 142590_25590 | 2.36871E−10 | B | C | T | 34,482,685 | 142590_17895 | 142590_28798 | 34,474,993 | 34,485,895 |
| 142590_33839 | 2.8199E−10 | B | G | T | 34,490,939 | un25356_46_47 | 140150_7178 | 34,488,198 | 34,517,886 |
| 140150_1360 | 1.92881E−09 | B | C | T | 34,523,417 | 140150_7178 | 130715_7900 | 34,517,886 | 34,528,538 |
| 349_17206 | 5.17564E−08 | A | G | C | 34,780,632 | 139757_2759 | 132316_31178 | 34,646,776 | 35,058,140 |
| 163336_1986 | 2.36871E−10 | A | C | T | 34,891,501 | 139757_2759 | 132316_31178 | 34,646,776 | 35,058,140 |
| 206_127177 | 6.65439E−07 | A | C | T | 35,311,416 | 206_129952 | 206_93069 | 35,308,641 | 35,380,077 |
| 136538_10981 | 3.69688E−07 | A | T | C | 35,380,437 | 206_93069 | 136538_12132 | 35,380,077 | 35,381,587 |
| 206_18683 | 2.47531E−08 | B | G | A | 35,484,450 | 206_22695 | 206_1735 | 35,480,428 | 35,501,301 |
| 206_7604 | 1.12514E−08 | B | G | T | 35,495,416 | 206_22695 | 206_1735 | 35,480,428 | 35,501,301 |
| 116563_668 | 2.36871E−10 | B | G | A | 35,510,063 | 206_1735 | 206_83762 | 35,501,301 | 35,590,249 |
| 139227_3125 | 1.92881E−09 | A | G | C | 36,403,557 | 137033_2731 | 141325_50733 | 36,356,165 | 36,439,598 |
| Cannabis.v1_{—} | 3.69688E−07 | B | T | C | 37,068,689 | 382_142922 | 382_113772 | 37,041,111 | 37,073,305 |
| scf4395- | | | | | | | | | |
| 18844_100 | | | | | | | | | |
| Cannabis.v1_{—} | 5.17564E−08 | B | A | T | 37,071,526 | 382_142922 | 382_113772 | 37,041,111 | 37,073,305 |
| scf4395- | | | | | | | | | |
| 21676_100 | | | | | | | | | |
| 382_17320 | 7.10614E−10 | B | G | A | 37,179,593 | 382_20744 | 382_7963 | 37,176,172 | 37,199,235 |
| 142190_9669 | 6.65439E−07 | A | T | C | 37,576,767 | 139626_2782 | 8242_520 | 37,555,539 | 37,614,785 |
| 141795_293547 | 1.16452E−06 | B | G | C | 37,674,639 | 141795_320990 | 141795_288050 | 37,652,457 | 37,704,840 |
| 141916_42986 | 2.36871E−10 | B | T | G | 37,925,069 | 141795_146748 | 141916_59905 | 37,826,687 | 37,942,022 |
| 141916_45118 | 2.36871E−10 | B | T | C | 37,927,201 | 141795_146748 | 141916_59905 | 37,826,687 | 37,942,022 |
| 107418_6807 | 1.03513E−07 | A | T | G | 38,043,498 | 141916_59905 | 82540_1522 | 37,942,022 | 38,045,943 |
| 141795_142649 | 2.8199E−10 | A | A | G | 38,175,429 | 82540_1522 | 134492_8727 | 38,045,943 | 38,243,031 |
| 141916_116856 | 1.03513E−07 | A | T | C | 38,298,835 | 134521_507 | 126320_2886 | 38,287,948 | 38,330,394 |
| 141795_61056 | 3.69688E−07 | B | G | T | 38,498,502 | 141795_68529 | 141795_54578 | 38,491,029 | 38,504,980 |
| 141795_29536 | 6.65439E−07 | B | C | T | 38,530,025 | 141795_35972 | Cannabis.v1_{—} | 38,523,589 | 38,533,292 |
| | | | | | | | scf4197- | | |
| | | | | | | | 25341_101 | | |
| 141795_15400 | 2.36871E−10 | B | G | A | 38,544,151 | 141795_22333 | 141795_13366 | 38,537,218 | 38,546,185 |
| 112471_1827 | 1.12514E−08 | B | A | T | 38,594,588 | 141795_13366 | 79621_13013 | 38,546,185 | 38,594,990 |
| 136139_6692 | 1.33945E−08 | A | A | G | 38,844,471 | 117370_1889 | 136139_9740 | 38,829,508 | 38,847,519 |
| 138966_2230 | 1.92881E−09 | A | G | A | 38,862,689 | 136139_9740 | 108017_1863 | 38,847,519 | 38,900,490 |
| 335_146423 | 1.99063E−07 | B | T | C | 39,073,782 | 335_127861 | 335_173131 | 39,067,308 | 39,093,591 |
| 335_163771 | 1.92881E−09 | B | T | C | 39,084,115 | 335_127861 | 335_173131 | 39,067,308 | 39,093,591 |
| 122117_134 | 1.16452E−06 | A | A | G | 39,097,992 | 335_173131 | 139304_4010 | 39,093,591 | 39,137,758 |
| 335_388188 | 6.65439E−07 | A | G | T | 39,359,130 | 335_387322 | 335_391708 | 39,358,264 | 39,362,650 |
| 335_412239 | 6.65439E−07 | A | C | G | 39,383,118 | 335_405852 | 335_425241 | 39,376,740 | 39,396,115 |
| 335_671981 | 1.16452E−06 | A | A | T | 39,921,599 | 335_667122 | 140982_191339 | 39,916,740 | 40,062,963 |
| 142714_17544 | 2.36871E−10 | A | T | C | 40,830,255 | 142714_14923 | 142714_21911 | 40,827,633 | 40,834,585 |
| 142714_45014 | 1.20834E−09 | B | C | T | 40,870,508 | 142714_21911 | 133548_2021 | 40,834,585 | 40,974,022 |
| 140223_9365 | 1.03513E−07 | B | T | C | 40,958,538 | 142714_21911 | 133548_2021 | 40,834,585 | 40,974,022 |
| 120094_8429 | 6.65439E−07 | A | G | T | 41,197,544 | 140223_25371 | 123629_3680 | 40,983,668 | 41,197,761 |
| 262_69417 | 6.65439E−07 | A | G | C | 41,307,507 | 262_56194 | 262_87020 | 41,295,132 | 41,327,013 |
| 262_562002 | 2.47531E−08 | A | C | G | 42,191,944 | 262_554112 | 262_578779 | 42,184,060 | 42,225,095 |
| 262_586008 | 1.13152E−06 | A | C | T | 42,229,455 | 262_578779 | 262_589844 | 42,225,095 | 42,233,263 |
| 136191_2918 | 2.47531E−08 | A | A | G | 42,396,589 | 262_690973 | 77159_4792 | 42,386,210 | 42,405,866 |
| 133169_3030 | 2.47531E−08 | A | A | G | 42,412,816 | 77159_4792 | 103133_5194 | 42,405,866 | 42,445,864 |
| 262_823098 | 2.47531E−08 | A | G | T | 42,508,652 | 262_814657 | 195138_1354 | 42,500,119 | 42,612,199 |
| 262_896805 | 2.47531E−08 | B | G | A | 42,603,366 | 262_814657 | 195138_1354 | 42,500,119 | 42,612,199 |
| 262_945202 | 2.47531E−08 | B | G | A | 42,665,152 | 262_943706 | 262_949888 | 42,663,656 | 42,669,838 |
| 131996_3293 | 2.8199E−10 | B | C | T | 43,047,034 | 115705_339 | 131996_2186 | 43,044,430 | 43,048,141 |
| 79535_985 | 6.65439E−07 | B | C | T | 43,215,274 | 90987_863 | 79535_6401 | 43,214,271 | 43,220,690 |
| 141930_7154 | 7.10614E−10 | B | T | C | 43,355,502 | 142560_10423 | 139379_3780 | 43,336,853 | 43,397,055 |
| 141930_19037 | 8.19942E−09 | B | G | A | 43,362,522 | 142560_10423 | 139379_3780 | 43,336,853 | 43,397,055 |
| 126003_3513 | 6.65439E−07 | A | T | C | 43,904,143 | 262_1277550 | 139833_37526 | 43,789,216 | 43,983,873 |
| 262_1391505 | 1.12514E−08 | A | A | T | 43,923,005 | 262_1277550 | 139833_37526 | 43,789,216 | 43,983,873 |
| 201851_1161 | 6.65439E−07 | A | T | C | 44,236,127 | 201852_1127 | 187075_4618 | 44,236,093 | 44,256,909 |
| 136535_4246 | 5.17564E−08 | A | G | T | 44,246,864 | 201852_1127 | 187075_4618 | 44,236,093 | 44,256,909 |
| 333_79521 | 5.25287E−07 | A | C | G | 44,262,185 | 187075_4618 | 333_73562 | 44,256,909 | 44,268,169 |
| 325_357603 | 7.9219E−07 | A | A | G | 45,191,090 | 325_401376 | 325_296054 | 45,149,827 | 45,258,496 |
| 325_69746 | 3.69688E−07 | B | G | A | 45,516,981 | 325_83710 | 325_57640 | 45,503,011 | 45,529,013 |
| 325_27548 | 6.65439E−07 | B | T | A | 45,562,350 | 325_29820 | 137380_180 | 45,560,069 | 45,610,057 |
| 325_25989 | 5.17564E−08 | B | C | T | 45,563,891 | 325_29820 | 137380_180 | 45,560,069 | 45,610,057 |
| 121785_197 | 6.65439E−07 | B | G | A | 45,592,056 | 325_29820 | 137380_180 | 45,560,069 | 45,610,057 |
| 325_22366 | 5.17564E−08 | A | A | C | 45,693,190 | 137380_4594 | 142606_18021 | 45,614,409 | 45,821,012 |
| 202805_380 | 6.65439E−07 | A | G | C | 46,397,576 | 202800_348 | 123771_2021 | 46,385,416 | 46,452,809 |
| 113322_2963 | 6.65439E−07 | A | C | T | 46,405,726 | 202800_348 | 123771_2021 | 46,385,416 | 46,452,809 |
| 79189_1859 | 7.9219E−07 | A | G | A | 46,474,244 | 140892_15988 | 135911_291 | 46,458,173 | 46,474,424 |
| 139441_12589 | 6.16147E−08 | B | G | A | 47,604,285 | 163_542168 | 139441_19680 | 47,225,704 | 47,611,349 |
| 141252_285 | 6.65439E−07 | A | G | T | 47,665,099 | 91478_3377 | 141252_10290 | 47,619,408 | 47,675,117 |
| 141252_7571 | 1.63949E−07 | A | T | C | 47,672,379 | 91478_3377 | 141252_10290 | 47,619,408 | 47,675,117 |
| 176378_2961 | 9.93466E−09 | A | T | G | 47,708,135 | 176377_4445 | 134813_9139 | 47,706,648 | 47,722,257 |
| 127130_1621 | 9.93466E−09 | A | T | G | 48,380,340 | 141521_5082 | 141314_1419 | 48,373,357 | 48,403,034 |
| 141314_10717 | 9.93466E−09 | B | T | G | 48,388,505 | 141521_5082 | 141314_1419 | 48,373,357 | 48,403,034 |
| 140347_5675 | 9.93466E−09 | A | C | G | 48,757,508 | 300_56425 | 140347_24146 | 48,670,445 | 48,785,137 |
| 140347_14739 | 1.16279E−08 | B | A | T | 48,920,367 | 187_418539 | 127012_2096 | 48,872,236 | 48,923,474 |
| 142473_9429 | 9.93466E−09 | A | T | A | 50,082,232 | 142473_5308 | 142473_18712 | 50,078,107 | 50,091,559 |
| 142473_23140 | 3.58437E−07 | B | T | C | 50,178,362 | 101079_1828 | 140878_27124 | 50,157,314 | 50,225,194 |
| 142473_62967 | 9.93466E−09 | B | C | T | 50,220,108 | 101079_1828 | 140878_27124 | 50,157,314 | 50,225,194 |
| 140878_17493 | 9.93466E−09 | B | T | C | 50,234,848 | 140878_27124 | 140878_10793 | 50,225,194 | 50,241,633 |
| 130851_737 | 1.16279E−08 | A | A | G | 50,877,604 | Cannabis.v1_{—} | 142083_59360 | 50,877,210 | 50,934,958 |
| | | | | | | scf2647- | | | |
| | | | | | | 9777_100 | | | |
| 142083_7626 | 3.69688E−07 | A | C | T | 50,909,707 | Cannabis.v1_{—} | 142083_59360 | 50,877,210 | 50,934,958 |
| | | | | | | scf2647- | | | |
| | | | | | | 9777_100 | | | |
| 142083_12897 | 2.77139E−08 | A | T | G | 50,914,980 | Cannabis.v1_{—} | 142083_59360 | 50,877,210 | 50,934,958 |
| | | | | | | scf2647- | | | |
| | | | | | | 9777_100 | | | |
| 142083_63394 | 2.36871E−10 | A | G | A | 50,943,468 | 112396_8287 | 141387_1507 | 50,935,181 | 51,048,910 |
| 126572_11492 | 9.93466E−09 | B | C | A | 51,285,462 | 126572_4649 | 137905_5785 | 51,278,652 | 51,298,985 |
| 127191_2870 | 1.36409E−08 | B | C | A | 51,285,752 | 126572_4649 | 137905_5785 | 51,278,652 | 51,298,985 |
| 407_17536 | 4.40105E−07 | B | T | C | 51,585,800 | 407_12140 | 407_31218 | 51,580,405 | 51,599,095 |
| 407_49674 | 1.63949E−07 | A | T | A | 51,729,989 | 156169_5308 | 123173_2141 | 51,727,061 | 51,740,800 |
| 123173_7013 | 2.77139E−08 | A | T | A | 51,745,672 | 123173_2141 | 407_96417 | 51,740,800 | 51,783,843 |
| 407_628721 | 7.01477E−08 | A | C | T | 52,506,950 | 407_623931 | 407_636544 | 52,502,161 | 52,516,871 |
| 407_658372 | 7.01477E−08 | A | A | C | 52,549,792 | 407_646369 | 407_664069 | 52,536,545 | 52,555,488 |
| 128941_12252 | 7.01477E−08 | B | G | A | 54,566,650 | 400_753229 | 139215_6866 | 54,554,494 | 54,616,328 |
| 400_32062 | 8.2059E−08 | B | G | A | 55,366,336 | 400_34357 | 120940_1506 | 55,354,710 | 55,382,920 |
| 138257_13217 | 7.01477E−08 | A | T | C | 56,490,139 | 138257_6041 | 105394_3643 | 56,482,961 | 56,506,984 |
| 138501_13604 | 7.01477E−08 | B | C | G | 56,660,721 | 105394_3643 | 140492_5857 | 56,506,984 | 56,675,274 |
| 114919_14527 | 7.01477E−08 | A | T | C | 56,968,116 | 141454_8267 | 114919_119 | 56,869,153 | 56,982,498 |
| 336_868 | 7.01477E−08 | A | T | C | 57,308,692 | 336_3841 | 121434_4727 | 57,305,719 | 57,315,434 |
| 81614_673 | 7.01477E−08 | B | G | A | 57,712,867 | 130556_7659 | 134336_4918 | 57,641,621 | 57,718,356 |
| 139966_17188 | 1.99063E−07 | A | A | G | 60,822,892 | 129357_878 | 139780_2541 | 60,802,746 | 60,863,054 |
| 348_380630 | 2.82847E−07 | A | T | C | 62,480,171 | 138837_1264 | 348_375955 | 62,478,303 | 62,490,054 |
| 131142_23300 | 1.16452E−06 | A | A | G | 63,128,832 | 348_322135 | 131142_22069 | 63,114,210 | 63,130,063 |
| 109648_2740 | 1.16452E−06 | A | C | T | 63,599,570 | 115082_11948 | Cannabis.v1_{—} | 63,593,153 | 63,648,497 |
| | | | | | | | scf5090- | | |
| | | | | | | | 2958_101 | | |
| 166_1394159 | 1.03513E−07 | B | C | T | 63,714,224 | 166_1420753 | Cannabis.v1_{—} | 63,675,477 | 63,721,208 |
| | | | | | | | scf1886- | | |
| | | | | | | | 705_100 | | |
| 141828_45835 | 2.47531E−08 | B | G | A | 63,921,961 | 166_1267707 | 141828_41817 | 63,869,881 | 63,925,983 |
| 166_982294 | 1.23229E−07 | A | G | C | 64,341,255 | 166_985249 | 166_976188 | 64,338,237 | 64,349,231 |
| 166_776344 | 1.03513E−07 | A | A | G | 64,547,738 | 166_787282 | 166_772518 | 64,528,809 | 64,552,554 |
| 135481_1166 | 1.23229E−07 | A | G | A | 65,036,575 | Cannabis.v1_{—} | 125670_6114 | 65,022,220 | 65,046,020 |
| | | | | | | scf1198- | | | |
| | | | | | | 99332_100 | | | |
| 275_491530 | 4.82202E−09 | B | T | C | 66,071,116 | Cannabis.v1_{—} | 163919_464 | 66,066,251 | 66,100,343 |
| | | | | | | scf5334- | | | |
| | | | | | | 1595_101 | | | |
| 275_335540 | 2.47531E−08 | B | C | G | 66,631,011 | 140615_1387 | 275_322068 | 66,619,677 | 66,645,982 |
| 101063_1888 | 2.47477E−11 | B | T | C | 66,775,861 | 275_210736 | 275_54258 | 66,770,826 | 66,804,557 |
| Cannabis.v1_{—} | 1.99063E−07 | A | C | T | 66,784,085 | 275_210736 | 275_54258 | 66,770,826 | 66,804,557 |
| scf5334- | | | | | | | | | |
| 1595_101 | | | | | | | | | |
| 135123_16024 | 1.99063E−07 | B | C | A | 66,885,379 | 275_26433 | 135123_25003 | 66,837,938 | 66,894,226 |
| 275_5858 | 6.9668E−11 | B | A | T | 67,272,033 | 275_48417 | 135123_18283 | 67,219,360 | 67,291,175 |
| 140521_35015 | 2.47531E−08 | B | G | C | 67,514,890 | 140521_21068 | 140521_37172 | 67,504,025 | 67,517,083 |
| 134455_36670 | 2.36871E−10 | B | C | T | 67,535,229 | 140521_43338 | 134455_30677 | 67,523,203 | 67,541,329 |
| 140222_4953 | 1.03513E−07 | A | C | A | 67,656,258 | 140222_13530 | 140222_573 | 67,647,768 | 67,660,655 |
| 300_48814 | 1.92881E−09 | A | T | C | 68,551,248 | 103758_170 | 300_25363 | 68,546,120 | 68,574,566 |
| 300_41986 | 1.92881E−09 | B | C | T | 68,558,021 | 103758_170 | 300_25363 | 68,546,120 | 68,574,566 |
| 300_37128 | 2.47531E−08 | B | T | C | 68,562,883 | 103758_170 | 300_25363 | 68,546,120 | 68,574,566 |
| 300_14533 | 6.9668E−11 | B | C | T | 68,592,104 | 300_25363 | 127413_28496 | 68,574,566 | 68,593,638 |
| Cannabis.v1_{—} | 1.12514E−08 | A | A | G | 68,721,246 | 138421_35937 | 138421_30699 | 68,719,948 | 68,725,186 |
| scf7554- | | | | | | | | | |
| 7601_101 | | | | | | | | | |
| 138421_25209 | 1.7417E−11 | B | C | T | 68,730,683 | 138421_30699 | 138421_20595 | 68,725,186 | 68,735,296 |
| 121193_2680 | 1.12514E−08 | B | T | G | 69,003,698 | 165_7042 | 123792_9056 | 68,922,097 | 69,033,540 |
| 141410_63603 | 1.3112E−11 | A | C | A | 69,072,463 | 145861_3311 | 141410_47266 | 69,061,049 | 69,088,801 |
| 136335_4018 | 1.3112E−11 | A | A | G | 69,236,641 | 134764_14714 | 134764_3655 | 69,226,040 | 69,236,985 |
| 136335_1169 | 1.3112E−11 | A | A | T | 69,239,452 | 134764_3655 | 182238_3757 | 69,236,985 | 69,258,864 |
| 182237_4521 | 1.3112E−11 | A | A | G | 69,305,092 | Cannabis.v1_{—} | 182237_901 | 69,278,729 | 69,308,712 |
| | | | | | | scf585- | | | |
| | | | | | | 250_100 | | | |
| 142164_9819 | 1.16452E−06 | A | G | A | 69,539,678 | 138744_7043 | 114370_3192 | 69,496,492 | 69,545,637 |
| 114370_3192 | 3.58045E−11 | A | T | C | 69,545,637 | 142164_9819 | 142164_20305 | 69,539,679 | 69,556,342 |
| Cannabis.v1_{—} | 3.58045E−11 | A | A | T | 69,678,995 | 316_16234 | 316_61777 | 69,630,166 | 69,681,764 |
| scf9865- | | | | | | | | | |
| 354_100 | | | | | | | | | |
| 142164_2682 | 3.69668E−07 | B | C | T | 70,364,873 | 139100_11454 | 136300_79178 | 70,249,641 | 70,520,851 |
| 165_10311 | 1.16452E−06 | B | G | T | 70,552,675 | 136300_79178 | 165_29950 | 70,520,851 | 70,572,298 |
| 165_168932 | 1.12514E−08 | B | G | A | 70,696,508 | 165_159921 | 165_192377 | 70,692,543 | 70,723,712 |
| 165_238452 | 5.17564E−08 | B | A | G | 70,769,733 | 165_235542 | 165_245701 | 70,766,823 | 70,777,023 |
| 127136_192 | 4.19057E−07 | B | C | T | 71,191,901 | 138743_6474 | 127136_8741 | 71,187,895 | 71,201,200 |
| 134161_3785 | 8.53433E−10 | B | C | T | 71,204,416 | 127136_8741 | 180550_364 | 71,201,200 | 71,204,760 |
| 133827_1355 | 8.53433E−10 | B | G | T | 71,213,884 | 180537_2830 | 139564_47269 | 71,212,077 | 71,253,877 |
| 139564_17542 | 2.69351E−11 | A | C | T | 71,283,642 | 139564_29461 | 139808_29916 | 71,271,690 | 71,359,842 |
| 244_70268 | 2.26029E−10 | A | C | T | 71,464,643 | 139808_39553 | 244_47445 | 71,375,213 | 71,504,627 |
| 142322_7230 | 2.26029E−10 | A | G | A | 71,476,054 | 139808_39553 | 244_47445 | 71,375,213 | 71,504,627 |
| 113372_971 | 2.69351E−11 | A | T | C | 71,716,668 | 140627_26569 | 140627_39787 | 71,707,590 | 71,727,529 |
| 140627_49834 | 2.69351E−11 | B | C | T | 71,737,576 | 140627_39787 | 141926_2591 | 71,727,529 | 71,837,480 |
| 130527_1052 | 2.69351E−11 | A | C | G | 71,840,991 | 141926_2591 | 141186_17297 | 71,837,480 | 71,951,074 |
| 138896_11705 | 2.69351E−11 | A | C | T | 71,902,441 | 141926_2591 | 141186_17297 | 71,837,480 | 71,951,074 |
| 243_53941 | 2.36871E−10 | B | A | G | 72,043,845 | 243_43271 | 243_54715 | 72,033,175 | 72,044,619 |
| Cannabis.v1_{—} | 1.85616E−10 | B | C | G | 72,047,815 | 243_54715 | Cannabis.v1_{—} | 72,044,619 | 72,049,804 |
| scf4349- | | | | | | | scf4366- | | |
| 7597_99 | | | | | | | 26923_100 | | |
| 161314_20775 | 7.40341E−07 | A | T | C | 72,220,564 | 161312_5722 | 161311_17093 | 72,168,949 | 72,247,687 |
| un70518_59_60 | 1.12514E−08 | B | G | A | 72,250,376 | 161311_17093 | 161315_16272 | 72,247,687 | 72,267,330 |
| 161311_13422 | 1.85616E−10 | B | T | C | 72,251,358 | 161311_17093 | 161315_16272 | 72,247,687 | 72,267,330 |
| 130028_3387 | 3.69688E−07 | B | A | T | 72,335,998 | 126886_4993 | 130028_6085 | 72,330,743 | 72,338,680 |
| 140727_6346 | 8.53433E−10 | B | A | G | 72,515,564 | 140727_10814 | 110164_5391 | 72,509,243 | 72,517,131 |
| 142028_5941 | 9.99405E−10 | B | C | A | 72,585,309 | 141601_12756 | 142028_10149 | 72,566,524 | 72,589,517 |
| 142098_41008 | 9.62105E−08 | B | G | C | 72,690,334 | 142098_9495 | 179171_1728 | 72,643,265 | 72,721,248 |
| 132276_12128 | 2.69351E−11 | B | C | T | 72,762,298 | 179171_1728 | 179170_45435 | 72,721,249 | 72,798,781 |
| 179169_32833 | 1.44136E−09 | A | C | T | 72,786,344 | 179171_1728 | 179170_45435 | 72,721,249 | 72,798,781 |
| 179169_59917 | 9.99405E−10 | B | C | A | 72,813,354 | 179169_55677 | 179170_59131 | 72,809,115 | 72,813,403 |
| 345_137849 | 9.93466E−09 | B | A | C | 72,856,290 | 141124_1912 | 345_120175 | 72,827,022 | 72,873,921 |
| 134723_3436 | 6.6549E−07 | B | A | G | 72,941,220 | 117277_6203 | 345_35141 | 72,904,378 | 72,973,354 |
| 203300_10277 | 1.5987E−08 | B | C | A | 73,173,850 | 203303_7072 | 79118_10657 | 73,172,917 | 73,182,678 |
| 118567_2609 | 5.7405E−09 | B | T | C | 73,250,920 | 247_900 | 247_18351 | 73,241,261 | 73,262,747 |
| 247_12293 | 1.7417E−11 | B | T | G | 73,256,718 | 247_900 | 247_18351 | 73,241,261 | 73,262,747 |
| 247_24227 | 1.03513E−07 | B | G | A | 73,268,790 | 247_18351 | 136670_6794 | 73,262,747 | 73,291,694 |
| 135670_1991 | 1.85616E−10 | B | T | A | 73,286,900 | 247_18351 | 135670_6794 | 73,262,747 | 73,291,694 |
| 121_563233 | 1.17889E−08 | A | G | T | 73,433,599 | 121_531793 | 131166_13901 | 73,366,820 | 73,483,248 |
| 121_573752 | 9.98245E−09 | A | A | T | 73,444,913 | 121_531793 | 131166_13901 | 73,366,820 | 73,483,248 |
| 121_598517 | 1.68021E−08 | B | A | G | 73,491,394 | 131166_13901 | 121_618608 | 73,483,248 | 73,509,920 |
| 121_649479 | 3.50177E−11 | A | A | T | 73,540,570 | 121_634632 | 171473_6761 | 73,525,854 | 73,580,085 |
| 121_655362 | 3.83983E−10 | A | A | G | 73,546,461 | 121_634632 | 171473_6761 | 73,525,854 | 73,580,085 |
| 182988_5645 | 1.68021E−08 | B | A | T | 73,581,205 | 171473_6761 | 125608_5344 | 73,580,085 | 73,627,903 |
| 171473_2079 | 3.83983E−10 | B | G | T | 73,584,768 | 171473_6761 | 125608_5344 | 73,580,085 | 73,627,903 |
| 141849_3893 | 1.09808E−09 | A | C | T | 73,820,614 | 141178_2275 | 129816_2540 | 73,799,827 | 73,840,855 |
| 141849_10928 | 2.90591E−09 | A | C | A | 73,828,244 | 141178_2275 | 129818_2540 | 73,799,827 | 73,840,855 |
| 114244_3895 | 2.90591E−09 | A | A | G | 73,847,393 | 129816_2540 | 141048_3173 | 73,840,855 | 73,896,283 |
| 359_250706 | 2.36871E−10 | B | T | C | 74,211,079 | 359_342846 | 359_236818 | 74,098,968 | 74,214,912 |
| 359_162248 | 1.16452E−06 | B | A | G | 74,312,211 | 359_167811 | 359_156134 | 74,306,648 | 74,318,325 |
| 129610_1197 | 3.69688E−07 | A | T | C | 74,465,573 | 129461_757 | 140382_18236 | 74,465,137 | 74,508,186 |
| 122547_1202 | 4.91029E−07 | B | T | C | 74,522,550 | 140382_25997 | 114607_4073 | 74,515,945 | 74,527,848 |
| 247_183551 | 9.99405E−10 | B | A | G | 74,602,627 | 78391_545 | 247_170489 | 74,564,905 | 74,615,708 |
| 123525_8741 | 2.90591E−09 | A | C | A | 74,698,144 | 136733_7450 | 121_446781 | 74,686,305 | 74,702,934 |
| 121_407892 | 7.90432E−08 | A | G | A | 74,742,025 | 121_426745 | 136615_6190 | 74,723,059 | 74,748,476 |
| 79290_437 | 7.19373E−09 | B | A | G | 74,744,031 | 121_426745 | 136615_6190 | 74,723,059 | 74,748,476 |
| 103334_6646 | 2.90591E−09 | A | T | C | 74,861,308 | 141677_13830 | 137322_15866 | 74,781,979 | 74,900,777 |
| 201948_3704 | 1.09808E−09 | A | T | C | 74,888,146 | 141677_13830 | 137322_15866 | 74,781,979 | 74,900,777 |
| 201975_771 | 3.63983E−10 | A | A | T | 74,893,445 | 141677_13830 | 137322_15866 | 74,781,979 | 74,900,777 |
| 73718_2463 | 3.83983E−10 | A | T | C | 74,938,563 | 137322_15866 | 131285_3785 | 74,900,777 | 74,956,520 |
| 121_402777 | 3.83983E−10 | A | G | T | 74,958,259 | 131285_3785 | 121_385289 | 74,956,520 | 74,976,464 |
| 121_396110 | 3.50177E−11 | A | G | C | 74,965,647 | 131285_3785 | 121_385289 | 74,956,520 | 74,976,464 |
| 121_379383 | 1.68021E−08 | A | C | T | 74,962,341 | 121_385289 | 121_374013 | 74,976,464 | 74,987,719 |
| 121_262981 | 4.35808E−09 | A | A | G | 75,136,633 | 139897_3033 | 121_254817 | 75,114,056 | 75,144,797 |
| Cannabis.v1_{—} | 2.62469E−11 | A | T | C | 75,137,014 | 139897_3033 | 121_254817 | 75,114,056 | 75,144,797 |
| scf3653- | | | | | | | | | |
| 22561_101 | | | | | | | | | |
| 121_257627 | 2.62469E−11 | B | A | G | 75,141,986 | 139897_3033 | 121_254817 | 75,114,056 | 75,144,797 |
| 121_250793 | 3.69688E−07 | B | G | A | 75,148,824 | 121_254817 | 121_245264 | 75,144,796 | 75,161,143 |
| 121_245264 | 1.37453E−08 | A | G | A | 75,161,143 | 121_250793 | 121_222469 | 75,148,825 | 75,183,953 |
| 121_232638 | 2.96619E−08 | A | C | A | 75,173,809 | 121_250793 | 121_222469 | 75,148,825 | 75,183,953 |
| 121_226633 | 2.62469E−11 | B | G | C | 75,179,788 | 121_250793 | 121_222469 | 75,148,825 | 75,183,953 |
| Cannabis.v1_{—} | 6.63685E−07 | A | C | T | 75,203,184 | 121_206858 | 121_196138 | 75,199,537 | 75,210,257 |
| scf6575- | | | | | | | | | |
| 12387_101 | | | | | | | | | |
| 121_179510 | 3.28113E−07 | B | A | G | 75,226,884 | 121_185268 | 138923_2703 | 75,221,128 | 75,331,510 |
| 121_168631 | 1.4702E−09 | B | T | C | 75,241,415 | 121_185268 | 138923_2703 | 75,221,128 | 75,331,510 |
| 131606_10137 | 2.62469E−11 | A | T | C | 75,253,891 | 121_185268 | 138923_2703 | 75,221,128 | 75,331,510 |
| 121_14306 | 1.17817E−10 | B | G | A | 75,392,086 | 121_25397 | 119072_183 | 75,381,930 | 75,394,191 |
| 139344_13547 | 2.96619E−08 | A | G | T | 75,480,618 | 128447_4452 | 139344_19511 | 75,446,517 | 75,489,161 |
| 133657_1563 | 1.54685E−06 | A | G | A | 75,509,717 | 139344_19511 | 142641_844 | 75,489,161 | 75,512,642 |
| 142641_24264 | 1.32349E−09 | A | G | A | 75,545,324 | 142641_20082 | 135638_1263 | 75,540,306 | 75,548,661 |
| 142641_37108 | 1.17956E−06 | A | C | T | 75,586,006 | 135638_1263 | 142641_42710 | 75,548,661 | 75,588,792 |
| 125260_2781 | 7.90822E−07 | B | C | A | 75,591,421 | 142641_42710 | 124337_165 | 75,588,792 | 75,600,552 |
| 221_4775 | 7.90822E−07 | A | A | G | 75,626,662 | 112301_14815 | 221_8196 | 75,606,629 | 75,630,090 |
| 221_178442 | 5.58092E−07 | A | G | A | 75,800,407 | 221_171521 | 221_182678 | 75,781,043 | 75,804,628 |
| 221_26618 | 1.17956E−06 | A | G | A | 75,932,398 | 146858_2306 | 123598_5715 | 75,910,836 | 75,949,184 |
| 131522_11633 | 2.49841E−07 | A | G | C | 76,104,437 | 131522_184 | 139416_13596 | 76,092,941 | 76,245,642 |
| 142106_184595 | 5.17564E−08 | B | T | C | 76,271,249 | 139416_13596 | 142106_179325 | 76,245,641 | 76,276,521 |
| 138166_616 | 2.36871E−10 | B | A | G | 76,430,984 | 168476_3262 | 100257_6956 | 76,427,633 | 76,438,529 |
| 138757_971 | 2.47531E−08 | B | C | A | 76,591,097 | 142096_690 | 138062_16438 | 76,543,359 | 76,598,192 |
| 126559_4797 | 1.63949E−07 | B | G | A | 76,793,466 | 141345_17045 | 119098_7557 | 76,745,413 | 76,803,207 |
| 237_78260 | 1.17956E−06 | B | C | T | 76,978,779 | 237_118135 | un77195_69_70 | 76,935,443 | 77,048,002 |
| 292_32669 | 3.69688E−07 | B | T | A | 77,232,337 | 292_24947 | 292_38256 | 77,224,618 | 77,237,924 |
| 292_71307 | 6.28622E−07 | B | C | T | 77,305,463 | un84357_64_65 | 292_94505 | 77,302,120 | 77,328,633 |
| 138590_27393 | 1.99063E−07 | B | T | A | 77,449,286 | 142242_48832 | 125807_267 | 77,396,670 | 77,492,830 |
| 138590_24666 | 2.07345E−11 | B | T | G | 77,452,033 | 142242_48832 | 125807_267 | 77,396,670 | 77,492,830 |
| 117866_3992 | 9.93466E−09 | B | T | C | 77,567,942 | 237_222135 | 141260_9487 | 77,557,791 | 77,591,135 |
| 141661_14499 | 7.90822E−07 | B | A | G | 77,770,079 | 127738_1041 | 141661_9964 | 77,767,257 | 77,773,033 |
| 138830_5794 | 5.58092E−07 | A | G | A | 77,858,300 | 138830_9542 | 139587_39489 | 77,854,553 | 77,886,225 |
| 130139_5055 | 2.89773E−11 | B | A | G | 78,614,606 | 123790_672 | 141579_17705 | 78,140,004 | 78,655,406 |
| 142292_88476 | 1.63949E−07 | B | C | T | 78,887,311 | un120782_49_50 | 142292_107001 | 78,874,996 | 78,905,923 |
| 141285_6389 | 9.93466E−09 | B | C | T | 79,024,693 | 134142_3107 | 140284_48469 | 79,008,048 | 79,064,199 |
| 132467_262 | 3.99027E−07 | X | C | A | 79,263,154 | 79523_1736 | 140133_38791 | 79,259,767 | 79,295,529 |
| 142137_28909 | 3.58437E−07 | A | A | G | 82,210,649 | 142137_23964 | 142137_34685 | 82,202,893 | 82,216,429 |
| 102_6216‡(2) | 7.09743E−08 | X | A | G | 85,807,792 | 109_2469 | 103_14301 | 85,779,583 | 85,816,304 |
| 141741_686746‡(3) | 1.18304E−06 | B | G | T | 78,519,130 | Cannabis.v1_{—} | 141741_719965 | 78,508,280 | 78,552,354 |
| | | | | | | scf646- | | | |
| | | | | | | 157885_101 | | | |
| 140896_105705‡(4) | 1.05601E−06 | X | C | T | 65,565,100 | 142498_814302 | 142498_803345 | 65,564,245 | 65,575,203 |
| 112460_513‡ (6) | 1.18304E−06 | B | T | A | 4,712,978 | 376_259037 | 376_225619 | 4,685,491 | 4,718,803 |
| 142100_1218219‡(6) | 1.67999E−08 | X | T | A | 14,621,523 | 142100_1236968 | 137736_52505 | 14,602,756 | 14,719,146 |
| 121977_562‡ (6) | 4.53393E−07 | X | C | A | 20,187,255 | 136155_40250 | 126714_3472 | 20,137,156 | 20,225,555 |
| 141466_2004‡ (6) | 3.84885E−07 | X | C | T | 27,006,811 | 236_42660 | 236_121558 | 26,941,568 | 27,030,117 |
| 141275_16934‡(6) | 1.05601E−06 | X | T | C | 49,434,383 | 140542_34647 | 114538_152 | 49,411,905 | 49,482,559 |
| 171_619793‡ (8) | 2.50049E−07 | X | G | T | 686,124 | 171_606839 | 171_624259 | 673,170 | 690,590 |
| 424_3562563‡ (9) | 4.32556E−11 | A | C | T | 8,228,671 | 424_3548700 | 424_3568489 | 8,214,850 | 8,234,582 |

First column, SNP marker name (markers are located on chromosome 1 except those indicated by ‡(x), in which cases the chromosome number is indicated within the parenthesis; Second column, p-value reflecting association of SNP markers with the autoflower phenotype identified in the two BSA analyses; Third column, genotype associated with the autoflowering phenotype (A = homozygous for reference allele, B = homozygous for alternative allele, X = heterozygous); Fourth column, reference allele call; Fifth column, alternative allele call; Sixth column, Abacus reference genome position; Seventh column, left flanking SNP of haplotype; Eight column, right flanking SNP of haplotype; Ninth column, Abacus reference genome position of left flanking SNP of haplotype; Tenth column, Abacus reference genome position of right flanking SNP of haplotype.

Validation of markers 166_325765 and 132604_11137 was performed through confirmation that the genotype associated with the autoflower phenotype was present in other, unrelated accessions with the autoflower phenotype and absent in photosensitive accessions. Autoflower varieties that were evaluated were AutoCBD (n=75 accessions) and Alaskan Yeti (n=1 accession). A total of 520 photosensitive varieties were evaluated. All autoflower accessions were homozygous for the alternative allele for both markers, whereas all photosensitive accessions were either heterozygous or homozygous for the reference allele for both markers, except for 15 photosensitive accessions that were homozygous for the alternative allele of marker 166_325765 and 3 photosensitive accessions that were homozygous for the alternative allele of marker 132604_11137.

Markers 132604_11137 and 166_325765 were further examined for three later flowering phenotypes (early, mid, and late). Table 3 shows that the autoflowering allele has a co-dominant inheritance with plants that are heterozygous for the markers flowering in general earlier as compared to plants that are homozygous for the reference allele not associated with the autoflowering trait.

TABLE 3
Analysis of the markers 132604_11137 and
166_325765 on the autoflowering group and three later
flowering groups observed in the set of F2s.

| 132604_{—} | 166_{—} | Autoflower | | | |
|---|---|---|---|---|---|
| 11137 | 325765 | (super early) | Early | Mid | Late |
| A | A | 0 | 2 | 24 | 13 |
| X | X | 0 | 30 | 12 | 2 |
| B | B | 20 | 0 | 0 | 0 |

“A” represents the homozygous reference allele, “X” represents heterozygous, and “B” represents the homozygous alternate allele, which is the genotype associated with the autoflowering trait.

Additional validation of markers 166325765 and 13260411137 was performed in 12 autoflowering seed lots (1-10 accessions per seed lot) as well as 63 photosensitive accessions representing 63 different seed lots used as controls. Additional accessions from two seed lots that were previously used for validations (Alaskan Yeti and AutoCBD) were included in this set. Genotype calls for the two SNP markers (166_325765 and 132604_11137) were recorded. All accessions with the homozygous alternate genotype for both SNP markers autoflowered (32-35 days to flower under 18 hours light) (Table 4). The two SNP markers thus are effective in 13 different genetic backgrounds (including the autoflowering accessions in the F2 populations that were used to map the two SNP markers in the first analysis) to predict the autoflower phenotype with 100% accuracy.

TABLE 4
Genotype calls (homozygous reference = A, heterozygous =
X, homozygous alternate = B) for the two preferred SNP markers
associated with the autoflower phenotype and days to flower (measured
in days after sow) for a set of commercially available autoflowering
seed lots that were evaluated under 18 hours light.

| | Number of | SNP marker | SNP marker | |
|---|---|---|---|---|
| | accessions | 132604_1117 | 166_325765 | Days to |
| Seed lot | tested | genotype(s) | genotype(s) | flower |
| Alaskan Yeti*** | 2 | B | B | 32 |
| Auto Pink | 1 | B | B | 35 |
| Kush***/**** | | | | |
| Chemdogging | 5 | A, X, B | A, X, B | 32-35 |
| Deimos | 2 | B | B | 32 |
| Dinafem | 3 | B | B | 35 |
| Auto**/*** | | | | |
| Dinafem Auto** | 7 | X | X, B | Not |
| | | | | flowering* |
| Hempfest | 2 | B | B | 35 |
| Autoflower** | | | | |
| Hempfest | 8 | X | X | Not |
| Autoflower | | | | flowering* |
| AutoCBD*** | 1 | B | B | 32 |
| S.O.D.K | 3 | B | B | 32 |
| Samsquatch OG | 3 | B | B | 32 |
| Auto | | | | |
| Solomatic**/*** | 10 | A, X, B | A, X, B | 35 |
| Sour Crack Auto | 3 | B | B | 32-35 |
| Walter White*** | 4 | A, X, B | A, X, B | 32-35 |

*Plants were observed up to 70 days after sow (DAS), if plants were not flowering at that time, they were marked as “not flowering.”
**Seed from selfed plants.
***Accessions used for gene sequencing.
****Accession was not part of the BSA.

Two seed lots (Dinafem Auto and Hempfest Autoflower) with heterozygous genotype calls for one or both SNP markers 166_325765 and 132604_11137 were associated with no flowering at the 10th week after onset of flowering. These heterozygous accessions started to pre-flower (some pistils visible) at week 8 after onset of flowering. In summer field conditions the same heterozygous genotype was associated with delayed flowering observed in the population of F2s that was used to map SNP markers 166_325765 and 132604_11137. These results indicate that a heterozygous genotype for the two SNP markers predicts delayed flowering in the field in summer with day length starting at 15.5 hours in June at sow, 15 hours late July at onset of flowering for the super early flowering group, 14.5 hours early August for the early/mid flowering group, and 14 hours mid August for the late flowering group. The heterozygous genotype thus causes an intermediate phenotype between autoflower and photosensitive in summer field conditions.

The 63 photosensitive accessions never flowered under 18 hours light. All 63 accessions had the homozygous reference allele for the two previously mapped SNP markers. This confirms previous observations for the F2s, where the homozygous reference genotype was associated with late flowering in a summer field setting. These plants flowered in the same timeframe as photosensitive varieties.

SNP markers 166_325765 and 132604_11137 are considered the preferred markers associated with autoflower because they show the highest level of association and resolution in the F2s, were confirmed in the second analysis and validated in multiple genetic backgrounds. In addition, because the two preferred markers are located in/near two genes for which Arabidopsis homologs are involved in the regulation of flowering time, it is believed that the causative genetic variation resides within or near one or both of these two genes. This indicates that the three autoflowering seed lots which segregate for the two preferred SNP markers have experienced one or more recombination events in the region between the two SNP markers and the causal genetic variant responsible for the autoflower phenotype.

The large size of the flanking region sharing the same haplotype with the two preferred SNP markers is expected to be the result of a single genetic source for the autoflower trait. This source was most likely introduced only recently in different genetic backgrounds allowing little time for recombination to break up the haplotype.

Because it is evident that in some genetic backgrounds one or more recombination events separated the preferred SNP markers from the causative genetic variant(s), additional SNP markers in marker assisted selection (MAS) efforts that make use of autoflowering germplasm that segregates for the two preferred SNP markers were desired. Therefore, 10 additional SNP markers which are flanking the two preferred SNP markers were identified for use in MAS. Four SNPs span a 401,807 bp region to the left of the two preferred SNP markers, whereas the other six SNPs span a 144,130 bp region to the right of the two preferred SNP markers. The haplotypes based on these 10 additional SNP markers together with the two preferred SNP markers were able to discriminate between all autoflowering and photosensitive accessions that were used in the second BSA with 100% accuracy (Table 5).

TABLE 5

| Ext. | 130617_{—} | 159_{—} | 138054_{—} | 159_{—} | 132604_{—} | 166_{—} | 122130_{—} | 166_{—} | 166_{—} | 109117_{—} | 340_{—} | 171326_{—} |
|---|---|---|---|---|---|---|---|---|---|---|---|---|
| haplotype | 9054 | 8752 | 8795 | 103549 | 11137 | 325765 | 2019 | 303719 | 297863 | 1157 | 14470 | 1256 |
| 1 | B | B | B | B | B | B | U | A | B | B | B | B |
| 2 | A | B | A | B | A | A | B | B | B | B | B | B |
| 3 | B | B | B | B | B | B | X | X | B | B | B | B |
| 4 | B | B | B | B | B | E | X | A | B | B | B | B |
| 5 | B | B | B | B | B | B | A | A | B | B | B | B |
| 6 | B | B | B | B | B | B | U | A | B | B | B | B |
| 7 | X | B | X | B | A | A | B | B | B | B | B | B |

Extended haplotypes based on 12 SNP marker haplotypes observed for all 12 autoflowering accessions that were used in the second BSA.
These 7 extended haplotypes can discriminate all 12 autoflowering accessions from all 63 photosensitive accessions.

The haplotypes described herein extend to all SNPs in the 579,814 bp region encompassing the two preferred SNP markers. Table 5 shows an example of haplotypes that can be observed for autoflowering accessions based on the genotypes observed for the data used in the second BSA. These haplotypes are not limited to what is represented in Table 5 as autoflowering accessions can also be heterozygous for the two preferred markers.

The two preferred markers are located in/near two genes. The first gene, which contains marker 166325765 (SEQ ID NO:41), has 73% homology to Arabidopsis gene AT2G39260 and is referred to as UPF2. The second gene, which is 50 Kb from marker 132604_11137 (SEQ ID NO:40), has 71% homology to Arabidopsis gene AT2G28550. This gene is referred to as RAP2.7 (related to AP2.7) or TOE1 (Target of Early Activation Tagged (EAT) 1). Since UPF2 acts together with UPF1 and UPF3 in a surveillance complex to activate NMD of mRNAs and because the NMD pathway is involved in the silencing of alternative splicing products of among others genes involved in the regulation of flowering time it is expected that a loss-of-function, reduced expression, or a UPF binding site changing-mutation in UPF2 would prevent or reduce activation of NMD and as a result alternative splice forms of flowering regulation genes would be in existence, potentially with result of an early flowering phenotype. Alternatively, the autoflower phenotype could be caused by a loss-of-function or reduced expression mutation of RAP2.7/TOE1 transcription factor, which as a result would no longer repress flower initiation, resulting in an early flowering phenotype.

### Example 2—Discovery of Cannabis Autoflower Genetic Variants

In order to identify naturally occurring genetic variants causing the autoflowering phenotype both candidate genes RAP2.7 and UPF2 were sequenced and evaluated for gene expression.

Gene expression analysis of the two candidate genes was done through RT-PCR. RNA was extracted from leaf tissue collected two weeks after onset of flowering from two photosensitive and two autoflowering accessions (Table 6; Nucleospin RNA Plant and Fungi kit, Macherey-Nagel). Leaf tissue was used for this experiment because it is believed that signaling events resulting in flower formation take place in leaf (Zhang and Chen 2021; PLoS Biology 19.2 (2021): e3001099).

Table 6. Accessions used for RT-PCR.

| Contextual ID | Alias | Seed lot | Type |
|---|---|---|---|
| PGTHR-429936 | AF1-1 | Auto Pink Kush** | Autoflower |
| PGTHR-429933 | AF3-1 | Dinafem Auto** | Autoflower |
| PGTHR-427093 | PS1-1 | Abacus | Photosensitive |
| PGTHR-427094 | PS2-1 | PAN-152* | Photosensitive |

*Not used for RT-PCR of UPF2.
**Grown from selfed seed.

After concentration adjustment and treatment with DNAse the RNA was used directly for RT-PCR (OneTaq® One-Step RT-PCR Kit, New England Biolabs). The Cannabis sativa homolog of the Arabidopsis ACT2 gene was used as a positive control. Primers used for RT-PCR can be found in Table 7.

RT-PCR results (FIG. 5) show that both RAP2.7 and UPF2 are expressed in both the autoflowering and photosensitive accessions, indicating that both genes are functional and expressed in leaf tissue during early flower development in both flowering types. In addition, RT-PCR results did not show a difference in gene expression between the autoflowering and photosensitive accessions for both RAP2.7 and UPF2, indicating that the autoflowering phenotype is not the result of expression differences in either candidate gene during early flower development.

The RAP2.7 genomic sequence was obtained through Sanger sequencing of genomic DNA (NucleoMag® Plant DNA extraction kit, Macherey-Nagel) from two autoflowering accessions (AutoCBD and Alaskan Yeti). Fragments 1 and 2 were sequenced from PCR product, fragments 3 and 4 contained heterozygous bases and were therefore sequenced after cloning (NEB PCR® Cloning Kit; New England Biolabs). Primers used for amplification and sequencing of fragments of the two candidate genes can be found in Table 7.

The coding sequence (CDS) for RAP2.7 was identified after alignment with Abacus (photosensitive) reference genome (version CsaAba2) genomic DNA sequence annotated and CDS. An amino acid sequence alignment shows a G (glycine) to E (glutamic acid) change at position 18 between photosensitive and autoflowering Cannabis, respectively, which is a non-polar to acidic amino acid change that could possibly affect gene function. This amino acid change is the result of a G to A base substitution at coding sequence position 53. In addition, compared to photosensitive Cannabis, there is a three amino acid deletion KLQ (lysine, leucine, glutamine) in autoflowering Cannabis starting at amino acid sequence position 35 compared to the photosensitive variant. This amino acid deletion is the result of a nine base deletion in autoflowering Cannabis of coding sequence AAACTGCAA between positions 103-111. Finally, there is a V (valine; nonpolar) to V/A (heterozygotic state; A=alanine; nonpolar) change identified at amino acid sequence position 253 between photosensitive and autoflowering Cannabis, respectively, caused by a T to TIC base substitution at coding sequence position 758.

The UPF2 coding sequence (CDS) was obtained through Sanger sequencing of cDNA (prepared from RNA using ProtoScript® II First Strand cDNA Synthesis Kit, New England Biolabs) and RT-PCR products from two autoflowering accessions (Tables 4 and 6) as well as through Sanger sequencing of genomic DNA from three additional autoflowering accessions (Table 4). Primers used for amplification and sequencing of fragments of the two candidate genes can be found in Table 7.

Sequences were aligned with the photosensitive Abacus reference genome (version CsaAba2) genomic DNA sequence and annotated CDS. The amino acid sequences were identical for the autoflowering accessions, but differed from the photosensitive variety for six amino acid substitutions: 1. A change at amino acid sequence position 21 from D in photosensitive (aspartic acid; acidic) to D/Y (heterozygotic state) in autoflower (Y=tyrosine; polar) caused by a nucleotide substitution of G to G/T at coding sequence position 61. 2. A change at amino acid sequence position 23 from C in photosensitive (cysteine; polar) to C/R (heterozygotic state) in autoflower (R=arginine; basic) caused by a nucleotide substitution of T to T/C at coding sequence position 67. 3. A change at amino acid sequence position 35 from E in photosensitive (glutamic acid; acidic) to E/G (heterozygotic state) in autoflower (G=glycine; nonpolar) caused by a nucleotide substitution of A to A/G at coding sequence position 104. 4. A change at amino acid sequence position 40 from H in photosensitive (histidine; basic) to HIC (heterozygotic state) in autoflower (C=cysteine; polar) caused by a nucleotide substitution from C to C/T at coding sequence position 118. 5. A change at amino acid sequence position 56 from G in photosensitive (glycine; nonpolar) to G/S (heterozygotic state) in autoflower (S=serine; polar) caused by a nucleotide substitution from G to G/A at coding sequence position 166. 6. A change at amino acid sequence position 1230 from Q in photosensitive (glutamine; polar) to P in autoflower (proline; nonpolar) caused by a substitution from A to C at coding sequence position 3689.

Table 7 provides a listing of sequences for the present invention.

TABLE 7

| SEQ ID NO | Description/SNP ID | Sequence |
|---|---|---|
| SEQ ID NO: 1 | 348_278501 | TACCCTGCGATTTGCTATGGTACTA |
| SEQ ID NO: 2 | 136501_10493 | AAAGGGTTTAATCTGTAAATATTGT |
| SEQ ID NO: 3 | 348_68337 | CTGGTGCTTCTGTGAGTTGACATTG |
| SEQ ID NO: 4 | 77102_2826 | AAAACGTTGCTAGCATGTATACTCA |
| SEQ ID NO: 5 | 78970_4740 | TTTTAATAAGCAAGAGTATTATAAC |
| SEQ ID NO: 6 | 130771_1619 | GTCCTTGGCCGTCTGGCTCTTCTAA |
| SEQ ID NO: 7 | 166_1420753 | AATTTATAATTAGTTATTAAATTTT |
| SEQ ID NO: 8 | 166_1344599 | GGTCATGAATTTTGCTAAGATTTGC |
| SEQ ID NO: 9 | 166_1342766 | TTCATCAAGTACGAAGATACAAATG |
| SEQ ID NO: 10 | 70692_112 | TATTATTATATCCGGATCATATGTA |
| SEQ ID NO: 11 | 137262_2355 | CTCCTTTTTATTTTTTGGTATAGGT |
| SEQ ID NO: 12 | 112864_918 | ACAGGGACTCCGTCTCAGAAGTGCG |
| SEQ ID NO: 13 | 141828_41817 | GTCAATACCTGGCCTCTATCATTTT |
| SEQ ID NO: 14 | 137089_3738 | TTTGGGTTTTAGGAAAAGGGATGAG |
| SEQ ID NO: 15 | 141828_26051 | ATATATGCAATTGCTGGATATGATT |
| SEQ ID NO: 16 | 166_1216813 | GGAAAAAATAAAAATTGAAGTAGGA |
| SEQ ID NO: 17 | 166_1210832 | GGACCCAACTTGCGCTTTACCTGGA |
| SEQ ID NO: 18 | 166_1072196 | GCACAATACACACCAACCTGAATAT |
| SEQ ID NO: 19 | 166_1050755 | TTGTAAACTAGTGTGTGAGAATGTT |
| SEQ ID NO: 20 | 166_1042556 | TAAGGCTACTTAATTATATTACTTC |
| SEQ ID NO: 21 | 166_1026787 | AACAATAAAATAAATTAGGATAATA |
| SEQ ID NO: 22 | 79036_402 | GGCAGCAGGTGGAGTAGGAGAAACT |
| SEQ ID NO: 23 | 104702_6585 | AGCTCTAACAGTTAGAGTTTTAAAG |
| SEQ ID NO: 24 | 104702_4384 | AAAGATTGGTCTAGCCTTTGTGTTT |
| SEQ ID NO: 25 | 166_976188 | GATTCTGTTTGCGACAGGCATTGAC |
| SEQ ID NO: 26 | 141264_16477 | CGGAAGAGGAGGGGAGGGGTCGGGG |
| SEQ ID NO: 27 | 126819_3234 | GGGCAGCAGCTAGTTCTAGCTTATA |
| SEQ ID NO: 28 | 166_800955 | TGATTTTGCATTCTCAACTTTCTTC |
| SEQ ID NO: 29 | 126791_897 | CAACCCTTTGTATACTTGGCTCCAC |
| SEQ ID NO: 30 | 166_684489 | GAATTATTTGAGCGAATATTATATA |
| SEQ ID NO: 31 | 101156_900 | CATTCATTCTAGCCTCAAAACTTTA |
| SEQ ID NO: 32 | 166_514787 | TCAAATCAAATTGATAAATTTCATG |
| SEQ ID NO: 33 | 118257_5997 | TATGGGCTTTGAGAAAATTGGCACA |
| SEQ ID NO: 34 | 166_408348 | GGAACTTGCTCGGCTTAGTGACATA |
| SEQ ID NO: 35 | 130617_9054 | TTCATCAGTGACCTGAATTGGTGAT |
| SEQ ID NO: 36 | 159_49348 | ATATGTTGAAGATGTGTCCGATTCC |
| SEQ ID NO: 37 | 138054_8795 | CCAACTGAAAAAGCTTGCTTGGTGG |
| SEQ ID NO: 38 | 159_79356 | CCGGTAACTTTGTCGTCGTCAGCAT |
| SEQ ID NO: 39 | 159_127948 | GTACAAATGGGCACTCATCAGTCAG |
| SEQ ID NO: 40 | 132604_11137 | AAATACACAAACTAATAGCTCGACT |
| SEQ ID NO: 41 | 166_325765 | CCTAATGTTTCTAATCTTTGTTTCA |
| SEQ ID NO: 42 | 166_303719 | TGAGTATGTAAATCATGTTTCTAAC |
| SEQ ID NO: 43 | 166_273040 | TCTGAAACTCAAGCCTCTCTGGGCC |
| SEQ ID NO: 44 | 166_250111 | CTTAGGGACCACCAATGTATCAAAG |
| SEQ ID NO: 45 | 340_14470 | GCAGCAGCACCCCCTTGCTTGAAAA |
| SEQ ID NO: 46 | 120836_5326 | CGGCGGAAGTGGAGGACGGTTCGGA |
| SEQ ID NO: 47 | 130163_5010 | TTGTTCTTGGGTATTAGAAGCAAGG |
| SEQ ID NO: 48 | 275_711038 | AGATGATTTTGTTAAACATTGTAGT |
| SEQ ID NO: 49 | 275_448497 | TTCATCTTCAACCCTATCATTATCG |
| SEQ ID NO: 50 | 132275_2738 | TTAGACTTGTGCTCCTTTGGATGCA |
| SEQ ID NO: 51 | 122921_3232 | GAACCTAGACCAGGCCAACCACAGG |
| SEQ ID NO: 52 | 211_215197 | CGAAAGGGGAAACAACTACGATATT |
| SEQ ID NO: 53 | 211_201852 | TCAACCTATAAATATAATTGTGTAT |
| SEQ ID NO: 54 | 211_198786 | TAGCCAAACCTACCAATTTGAATGC |
| SEQ ID NO: 55 | 177642_4242 | ACAAAGGTGTTTGTCAATGTAATGA |
| SEQ ID NO: 56 | 211_126528 | TTAACTATGGCCTGCAGGTCAATTC |
| SEQ ID NO: 57 | 211_60979 | ACACTTTACTTAGTATATAATAGAT |
| SEQ ID NO: 58 | 211_40813 | TGTCTCAGAGACGACAAGAATGTCT |
| SEQ ID NO: 59 | 211_14204 | ATGTGCCCGAAAAGCTATAATTTCA |
| SEQ ID NO: 60 | 79134_4041 | GAGGAGAATCAGTTGGTTTTCAAGG |
| SEQ ID NO: 61 | 300_332172 | GCAACCATAGACATTGGATAACTTG |
| SEQ ID NO: 62 | 141037_9199 | TATACAATGCCAGGCACATCCCAGC |
| SEQ ID NO: 63 | 157129_5206 | ATTAAGATAATAGATCACTGATGGC |
| SEQ ID NO: 64 | 142410_24821 | GTTGGAAGCCTCGGGGGCACCGGAA |
| SEQ ID NO: 65 | 141410_24865 | AAGATATTAACACTGCGGATTGGAT |
| SEQ ID NO: 66 | 135301_3997 | CCAAATCACCATGTGCAACACCCCA |
| SEQ ID NO: 67 | 125861_674 | ACATAGGGTCTGAGATTGTCGTTCG |
| SEQ ID NO: 68 | 182237_22983 | AGGCTTATCCTTGGACGCCTTTCTT |
| SEQ ID NO: 69 | 91363_1648 | ATGACATTGTCCTTAAGCTTGGGAC |
| SEQ ID NO: 70 | 171614_7078 | TTGCCGTATTTGTAATTAGTTTTAG |
| SEQ ID NO: 71 | 139100_11454 | TGAACTGGGCTCGCACATTCTTTTT |
| SEQ ID NO: 72 | 165_38630 | CTCTTTTTTCTTGCATGAATCCCTC |
| SEQ ID NO: 73 | 165_43011 | CTTTTATAAATTCCTGTGTCTCTTG |
| SEQ ID NO: 74 | 100294_1649 | GATATTTACAATGATTTATATAGTT |
| SEQ ID NO: 75 | 121703_4932 | TTCATACAATAGGTTGGATTGCAAT |
| SEQ ID NO: 76 | 165_61499 | GAAAGAATGTTATAAAATTTACCTG |
| SEQ ID NO: 77 | 165_76110 | ATGGCCTGAGTTTTCCAACCTCGTT |
| SEQ ID NO: 78 | 121858_4181 | CGGCGGAGATGAATGAGTATTAGAA |
| SEQ ID NO: 79 | 228_36608 | GGTTCTGATCGTCGTGATGGGAAGT |
| SEQ ID NO: 80 | 165_523583 | GAAGGATGCCCCTAGGAGGCACCGA |
| SEQ ID NO: 81 | 165_527008 | GAACCGTGATTTCCTCATTGGTTGC |
| SEQ ID NO: 82 | 133827_10943 | CCTTTCAACATACTACTTCCACCTT |
| SEQ ID NO: 83 | 244_4611 | TGGTTCAGCGAGTTCCTGAACCATT |
| SEQ ID NO: 84 | 140627_3175 | AGGATTCCCTCTCTGCGTCTAACTC |
| SEQ ID NO: 85 | 113372_17427 | TATTATAAATGACCCAATAATATCT |
| SEQ ID NO: 86 | 100680_1353 | AACAGAGGTATTGAAAGGGAAGCCC |
| SEQ ID NO: 87 | 122185_1743 | TGCAAGGAAGTTTGCTCTTTGCATC |
| SEQ ID NO: 88 | 138896_55595 | AGACAATGGTGTCGAGAACCCATCG |
| SEQ ID NO: 89 | 140727_152015 | AAGTTATTTAATTACAATAAGTATT |
| SEQ ID NO: 90 | 190653_3284 | TAGGGGCCTTATATGACAGCGCTTA |
| SEQ ID NO: 91 | 190652_1885 | TTCCATCTAACCTAAGAGTACAAAC |
| SEQ ID NO: 92 | 131552_1374 | TAAAATTTATTAGCCTCCGAAGAAA |
| SEQ ID NO: 93 | 121_580459 | GTTAGTCCTCACTCCAGGAGCTTTT |
| SEQ ID NO: 94 | 121_626183 | ATGAATCAAACTAAGCATAATTTAA |
| SEQ ID NO: 95 | 141849_964 | TTCTTGTTTGAAATTGGGGTTAAAC |
| SEQ ID NO: 96 | 141849_8876 | TGTTTTATGTTTGGTCTTACCTTAG |
| SEQ ID NO: 97 | 129816_7004 | TGCCTGATATGTGCATAGCACACAC |
| SEQ ID NO: 98 | 141048_10203 | GATGGTAATTGGTTGTCCTCCTCAT |
| SEQ ID NO: 99 | 204_79850 | TATTGGTTTACTTGCTGAAGCCCAA |
| SEQ ID NO: 100 | 141677_8625 | ACTAGCCACACTAGAAAGCCTTGAT |
| SEQ ID NO: 101 | 91362_6436 | AAAGGTTTAGCTCGAGTGTCATCTG |
| SEQ ID NO: 102 | 292_136433 | TTGATGAGGGAGCAAAATACTTTTC |
| SEQ ID NO: 103 | 300_84463 | AGGTGGATATCTCACTACAGATAAG |
| SEQ ID NO: 104 | 348_160959 | AACATGATTCCTAATAGATTCACCT |
| SEQ ID NO: 105 | 348_157790 | ACAGAGGATATAATACAGGTTTTGG |
| SEQ ID NO: 106 | 100933_1600 | TACCTCTCGATCGCCTTCAATGCAT |
| SEQ ID NO: 107 | 348_4479 | TCCTCCTAATGCGACCCACTTGATT |
| SEQ ID NO: 108 | 137952_1704 | GGAAGTTACTCCCGGAGGCCATTGA |
| SEQ ID NO: 109 | 133211_9562 | ATCTAAGATCCTGGTAAAATATATA |
| SEQ ID NO: 110 | Cannabis.v1_scf1886-705_100 | GTGAAGTTGTTTAATGAGTTTTAAA |
| SEQ ID NO: 111 | Cannabis.v1_scf1886-3142_101 | ATGTTCCACAATCCCTAAAACATTT |
| SEQ ID NO: 113 | Cannabis.v1_scf3513-33786_101 | GCTTGTAACAAAGCATTTAATATTT |
| SEQ ID NO: 114 | 130617_11900 | GTGCTCATGCCTCAAATGAAGCTAA |
| SEQ ID NO: 115 | 159_2273 | GCCAAGTCCTCAGCATGGTAATCTA |
| SEQ ID NO: 116 | 159_17477 | ATCCATTTTCCAGGTATAGGCTGGC |
| SEQ ID NO: 117 | 159_41757 | ATGACGTAATTTGTCTCCAGTAATG |
| SEQ ID NO: 118 | 138054_6707 | ATATGTTGAAGATGTGTCCGATTCC |
| SEQ ID NO: 119 | 275_764127 | TCATGGAATCTAAAAGGGAATCGAG |
| SEQ ID NO: 120 | 275_642249 | TTATTCCAACTTAAACAGATTAAGT |
| SEQ ID NO: 121 | 275_605120 | GTTTCAATGGTCTAAGTTCGTATCA |
| SEQ ID NO: 122 | 275_599093 | AGTGGGATTTATGGCAGGCCTAGCA |
| SEQ ID NO: 123 | 275_581421 | GGCAACTCAAAGGCAGAGATTGTCC |
| SEQ ID NO: 124 | 141029_14247 | TTAACTTGTCTCCACATGTGACATG |
| SEQ ID NO: 125 | 141029_9389 | TAGATTGGGTCACATTTTTGAAACA |
| SEQ ID NO: 126 | 79111_346 | TGTATATAGCACGAAATGTTACTTT |
| SEQ ID NO: 127 | 105272_527 | CCTACATCTACATATTGGGATGCAT |
| SEQ ID NO: 128 | 165719_1761 | TCCCTAACATCTTTAATGTGCTTGA |
| SEQ ID NO: 129 | 275_458039 | CGTACAAAATTCCTCACTGTACGCC |
| SEQ ID NO: 130 | 275_302105 | TTAGGATCTATTCTAATTTAGATCC |
| SEQ ID NO: 131 | 139438_4208 | TCCCATGATCGTGACGCTCCATTCA |
| SEQ ID NO: 132 | 275_117110 | TTCCCTTTCTCAATATGTATTTAAC |
| SEQ ID NO: 133 | 275_33664 | AACAGGAGAAGATAAATTAAGAATA |
| SEQ ID NO: 134 | 141279_11648 | AACCCCAGAAACTGCTCTCTAAAAT |
| SEQ ID NO: 135 | 275_309233 | CGGGGGTGATGTCTGCGACTGTCTT |
| SEQ ID NO: 136 | 275_126666 | ATCATCACTCTTGTCTTTTTTCTTT |
| SEQ ID NO: 137 | 139608_9837 | TTTATTTATTATCCTAGTCTTCAAG |
| SEQ ID NO: 138 | 140521_15210 | GTGAAGCTCACTCAAACTAGATGGT |
| SEQ ID NO: 139 | 140258_1471 | ATGTACATTAATTATGAATAGAACC |
| SEQ ID NO: 140 | 238_1215 | GAAAATCACCGTGAGGAGTGGGGTT |
| SEQ ID NO: 141 | 238_24099 | GCCAGGAGAGAGGTTACTGATACTA |
| SEQ ID NO: 142 | 211_30316 | AATCTCTTCTTTAGTTTGTTTCATT |
| SEQ ID NO: 143 | 139467_31539 | GATTGTTATTATTATTTTATAAACT |
| SEQ ID NO: 144 | 142410_20135 | TTTAAGGAGAGAGATCGACCATTTT |
| SEQ ID NO: 145 | 300_117302 | CCTCACCATCAGAAGGTACCTCACC |
| SEQ ID NO: 146 | 102355_181 | TGACCTTGAGAAGAAATCTCCCACC |
| SEQ ID NO: 147 | 300_32251 | AGCTGGGTTTTCCTCAAGCGAAGTT |
| SEQ ID NO: 148 | 181985_1530 | ATAACCTAGCTTGTTGAGGTCTTTT |
| SEQ ID NO: 149 | 181984_9517 | GCCCCAGCAACCGTTGTATTCTCCT |
| SEQ ID NO: 150 | 165_18641 | TATTTTTGAAGGGTTATCAAATCTC |
| SEQ ID NO: 151 | 101368_913 | TGAGATTCTTCAAAGAATAACACCA |
| SEQ ID NO: 152 | 120315_3015 | CAAACATTTTTCGATAAGTATACCT |
| SEQ ID NO: 153 | 171616_8850 | TTTAATTAAATTCAATTAATTAAGT |
| SEQ ID NO: 154 | 171614_23426 | TCCCACATATACCTGCCCAGTTCTT |
| SEQ ID NO: 155 | 138744_7043 | ATAATCAAAAGTGTCATCTAAGACA |
| SEQ ID NO: 156 | 142164_25210 | ATAACCCAATTTTATGGTGATTCCT |
| SEQ ID NO: 157 | 142384_11837 | AGCCCATTGGTACGAATAATTTGAA |
| SEQ ID NO: 158 | 139190_27380 | GCTGTTGTAAGATATTGGCAAGGTA |
| SEQ ID NO: 159 | 142164_495 | TGTGAGCATCCACAAACAAATTAAT |
| SEQ ID NO: 160 | 115119_7254 | TGAAACATTTCTATATTTGGGGTTG |
| SEQ ID NO: 161 | 221_24913 | TGCAACTTTGTAGAAAAGGTCTTTT |
| SEQ ID NO: 162 | 121_398155 | TTCTAACCACTGTACAAGGTTATAT |
| SEQ ID NO: 163 | 159_74552 | GTTACTAAATGTGCAACATATTTAT |
| SEQ ID NO: 164 | 275_654722 | AATGTCCAAGCACGCAACATCTCCA |
| SEQ ID NO: 165 | 275_564391 | AAGCTTGATATAAAGGGAAGCCTCT |
| SEQ ID NO: 166 | 159_8752 | ATCCATAGGCACAGCATCCTCATTC |
| SEQ ID NO: 167 | 159_103549 | GCAGGAAATGAAGTCGGAATATCCA |
| SEQ ID NO: 168 | 122130_2019 | TACACTTTGAAAAGAAGAATTAAAA |
| SEQ ID NO: 169 | 166_297863 | TACGACAAGCCGCGAGCACGAATAT |
| SEQ ID NO: 170 | 109117_1157 | TGTGACACTTTAATTTTTACAAAAA |
| SEQ ID NO: 171 | 171326_1256 | GGACGAGTCAACAACAGAGATGGGA |
| SEQ ID NO: 172 | C_RAP2.7 1F | GCCGATTCAACCTACGGGAA |
| SEQ ID NO: 173 | C_RAP2.7 1R | CTTGCAGCCTCTAGTTCGCT |
| SEQ ID NO: 174 | U2C_5F | TCAGCAAATGGACAGAGTGC |
| SEQ ID NO: 175 | U2C_5R | ATTCCACCACCGGAATAATG |
| SEQ ID NO: 176 | ACT2_1F | GAAGGCTGGTTTTGCTGGTG |
| SEQ ID NO: 177 | ACT2_1R | TCAGCAATGCCAGGGAACAT |
| SEQ ID NO: 178 | RAP2.7_frS_2F | CCAAACTAGATAGATTATTCTTCTGCC |
| | primer | |
| SEQ ID NO: 179 | RAP2.7_frS_2R | AACAACCGAAGAACCAGAGGA |
| | primer | |
| SEQ ID NO: 180 | RAP2.7_frA_1F | CGATTCAACCTACGGGAAGA |
| | primer | |
| SEQ ID NO: 181 | RAP2.7_frA_1R | CAGCATGAGCTGTGTCGAAT |
| | primer | |
| SEQ ID NO: 182 | RAP2.7_frB_2F | AGGTGGATTCGACACAGCTC |
| | primer | |
| SEQ ID NO: 183 | RAP2.7_frB_2R | CCAAGCCAAAGGCATAATGT |
| | primer | |
| SEQ ID NO: 184 | RAP2.7_frC.2_2F | ATTCGACAGCGAACTAGAGG |
| | primer | |
| SEQ ID NO: 185 | RAP2.7_frC.2_1R | ACAAGGGAGGCATTCAGAGA |
| | primer | |
| SEQ ID NO: 186 | U2C_1F primer | TGCATGGACTATAATGGTGAGTG |
| SEQ ID NO: 187 | U2C_1R primer | GGCATCACAAATGGAAGTCA |
| SEQ ID NO: 188 | U2C_2F primer | ACACTTCGGCAGAGCAATTT |
| SEQ ID NO: 189 | U2C_2R primer | GGTCTGATTCAGGTGCCAAT |
| SEQ ID NO: 190 | U2C_3F primer | GGGAAGCAGAATCCAAGATG |
| SEQ ID NO: 191 | U2C_3R primer | AAGCCAGCAGTGAGAGAAGC |
| SEQ ID NO: 192 | U2C_4F primer | GCTGAGGCAGCTTCGTAAAT |
| SEQ ID NO: 193 | U2C_4R primer | CGCTTCCAGAATCACTGTCA |
| SEQ ID NO: 194 | U2C_5F primer | TCAGCAAATGGACAGAGTGC |
| SEQ ID NO: 195 | U2C_5R primer | ATTCCACCACCGGAATAATG |
| SEQ ID NO: 196 | Predicted Cannabis | MTSMDHIFICMDYNGEWKITDNCIWEWFGTGCNK |
| | UPF2 protein | EFVVDHSIKFHQLVNKVYEKIGVDQNLYKIEITHKV |
| | sequence | AGETFNKMVPSKICGDSDVEDLLKELYKVKEVIPL |
| | | YVCIKKNNDKGKTKFVNDDDGDGNELTGDDVEFD |
| | | CDDDVFDNRSFYDNYFGCHIIDQVPNDPSYILNED |
| | | IPQSGEGIIGSNPTPDDIVHESGNNELQECDKVVE |
| | | ENNDVIENDIIVEGGQIIPYQHYDMFMGNVAQCSA |
| | | RGGAHQLRFSGCMMEVSVCEVKGKMDHHEDEG |
| | | RVGGGENIGKQNEEEAVARLEEMKKSIEGKITLRQ |
| | | SNLNPERPDSGFLRTLDSSIRRNTAVIKKLKQINEE |
| | | QREGLLDDLRSVNLSKFVSEAVTSICDAKLRSSDI |
| | | QSAVQICSLLHQRYKDFSPSLVQGLLKVFFPGKSG |
| | | DDSDTDRNQKAMKKRSTLKLLLELYFVGVIEDSAI |
| | | FVSIIKDLTSIEHLKDRDTTQTNLSLLASFARQGRIF |
| | | LGLPLSGQEVYEEFLKGLNITSDQKKIFRKALHAYY |
| | | EAASELLQTEHTSLRQLEHENAKILNAKGELSDEN |
| | | VASYEKLRKSYDQLYRNISSLAEALDMQPPVMPE |
| | | DGHTTRVTSGDDASSNSTGKDSSALEAIWDDEDT |
| | | RSFYECLPDLRAFVPAVLLGEAESKMNEQSVKTQ |
| | | EQSTELAPESDQVQQTAPDSAEISTDSGASQEGR |
| | | STEKGKEKEEKEKDKSKDPEKEKGKEKDADKKG |
| | | DTEKEKLKSIEGTNLDALLQRLPGCVSRDLIDQLTV |
| | | EFCYLNSKASRKKLVRALFNVPRTSLELLPYYSRM |
| | | VATLSTCMKDVSSMLLQMLEEEFNFLINKKDQMNI |
| | | ETKIRNIRFIGELCKFKIAPAGLVFSCLKACLDDFSH |
| | | HNIDVACNLLETCGRFLYRSPETTVRMANMLEILM |
| | | RLKNVKNLDPRHSTLVENAYYLCKPPERSARIAKV |
| | | RPPLHQYIRKLLFSDLDKSTIEHVLRQLRKLPWSE |
| | | CEPYLLKCFMKVHRGKYGQIHLIASLTAGLSRYHD |
| | | EFAVAVVDEVLEEIRVGLELNDYGMQQRRLAHMR |
| | | FLGELYNYEHVDSSVIFETLYLILVFGHGSPEQDLL |
| | | DPPEDCFRMRMVITLLETCGHYFDRGSSKRKLDR |
| | | FLIHFQRYILSKGALPLDIEFDLQDLFADLRPNMTR |
| | | YSSIEEVTLALVELEEHERTLPSDKTSSEKHSDSEI |
| | | RSSFNSISANGQSAVNGNEGNGRLHDGLGDSDS |
| | | DSGSGTLDQEGRDEEELDDENHDEECDTDEEDD |
| | | DGGGPASDEDEVHVRQKVMEVDPLEAATFEQEL |
| | | KAVMQESMDQRRQELRGRPTLNMMIPMNVFEGS |
| | | TRGVGGESGDEALDEEGGGIKDVQVKVLVKRGS |
| | | KQQTRQMYIPRDCSLVQSTKQKEAAELEEKQDIK |
| | | RLVLEYNDREEEELNGLGTQTLNHMQGSGSRGS |
| | | TRGHLWEGSSGRGGTRHRHYSGGGIFYNRKK |
| SEQ ID NO: 197 | Predicted Cannabis | MMLDLNVNINNGADSTYGKTKERGAELVIMEVED |
| | RAP2-7/TOE1 | KLQKGSTTQIMEDSGSSGSSVVNVEIDALSSTTTI |
| | protein sequence | TTSNGVFREEDSSINVTNTTSSTFFFDIMKREKDC |
| | | NNGATAGKETNNISPPGFLTRSFFPVAGEKVGNQ |
| | | FVEAGSGSSSSRPQWLNLSFADSGGGGAAVQPP |
| | | ADVKVLQQKQQIKKSRRGPRSRSSQYRGVTFYR |
| | | RTGRWESHIWDCGKQVYLGGFDTAHAAARAYDR |
| | | AAIKFRGVDADINFNVTDYDEDMMQMKNLSKEEF |
| | | VQILRRQSTGFSRGSSKYRGVTLHKCGRWEARM |
| | | GQFLGKKYIYLGLFDSELEAARAYDKAAIKQNGRD |
| | | AITNFEHSTYQGEIILDTNAQGNDHNLDLNLGISPP |
| | | CDGPKGREYSFGLGDTRVHWNPREGPHRIRPIMI |
| | | DGQSSPHILPPNHAASATCSGVYPAFLPKHEEMR |
| | | AMADDHQKRIEAANSSQGFLNWAWKIHGNGSNT |
| | | TTTTTSSCVTAMPTFSIAASSGFSSSTSLAALSATN |
| | | NINPQANFVQNNICLSPSMPITTTNSVTNNFHNSQI |
| | | HRG |

### Example 3—Discovery of Cannabis Autoflower Genetic Variants

Potential candidate genes in the QTL region located between 63,161,656-71,980,891 bp on chromosome 1 of the Abacus reference genome (version CsaAba2) were identified by exploring gene deserts for genes present in the CBDRx reference genome (version cs10) with annotations relevant to the autoflowering phenotype. The CBDRx genome was used to complement incomplete assembly in the Abacus reference genome. The largest gene desert in the Abacus reference genome in this QTL region spans 133.1 kb between positions 65,613,018-65,746,074 bp on chromosome 1. The CBDRx reference genome includes a homologous sequence of this gene desert region, which was identified by performing a BLASTP in NCBI of genes flanking the gene desert in the Abacus reference genome starting with UPF2, followed by genes located between UPF2 and the gene desert. The homolog of Regulator of nonsense transcripts UPF2 (UPF2; At2g39260, 65,451,912-65,485,728 bp on chromosome 1 of the Abacus reference genome) on the CBDRx reference genome is LOC115706264 (located between positions 19,802,609-19,815,150 bp on chromosome 1). The gene next to UPF2 on the Abacus reference genome is Zinc finger CCCH domain-containing protein 21 (C3H21; At2g20280; 65,487,998-65,491,263 bp on chromosome 1 of the Abacus reference genome), its homolog on the CBDRx reference genome is LOC115706080 (located between positions 19,843,513-19,847,204 on chromosome 1). The gene next to C3H21 on the Abacus reference genome is Protein TONNEAU 1a (TON1A; At3g55000; 65,506,744-65,510,360 bp on chromosome 1 of the Abacus reference genome), its homolog on the CBDRx reference genome is LOC115703871 (located between positions 19,860,264-19,863,668 bp on chromosome 1). The gene next to TON1A on the Abacus reference genome is UDP-XYL synthase 6 (UXS6; AT2G28760; 65,542,511-65,551,246 bp on chromosome 1 of the Abacus reference genome) its homolog on the CBDRx reference genome is LOC115705371 (located between positions 21,744,345 . . . 21,748,826 on chromosome 1). The gene next to UXS6 on the Abacus reference genome is UDP-XYL synthase 6 (UXS6; AT2G28760; 65,575,447-65,578,498 on chromosome 1 of the Abacus reference genome) its homolog on the CBDRx reference genome is LOC115705568 (located between positions 21,707,280 . . . 21,711,213 bp on chromosome 1), The gene next to UXS6 is Methionine-tRNA ligase, chloroplastic/mitochondrial (OVA1; At3g55400; 65,580,974-65,587,034 bp on chromosome 1 of the Abacus reference genome), its homolog on the CBDRx reference genome is LOC115707768 (located between positions 23,803,018 . . . 23,809,314 bp on chromosome 1). The CBDRx reference genome contains four additional genes in this region; LOC115705128 (two-component response regulator-like PRR37; located between positions 19,985,933-19,992.033 bp), LOC115705129 (unannotated; located between positions 19,988,482-19,992,665 bp), LOC115704703 (TBC1 domain family member 8B; located between positions 20,010,950-20,018,438 bp), and LOC115705441 (CDP-diacylglycerol—glycerol-3-phosphate 3-phosphatidyltransferase 2; located between position 20,032,520-20,036,951 bp).

Of the genes present in the CBDRx reference genome that are absent in the Abacus reference genome, PRR37 is the only gene that is involved in regulation of flowering time and therefore a candidate gene for further analysis of its alleles in autoflowering and photosensitive accessions (Klein, R. R. et al. (2015) ‘Allelic variants in the PRR37 gene and the human-mediated dispersal and diversification of sorghum’, Theoretical and Applied Genetics, 128(9), pp. 1669-1683; Murphy, R. L. et al. (2011) ‘Coincident light and clock regulation of pseudoresponse regulator protein 37 (PRR37) controls photoperiodic flowering in sorghum’, Proceedings of the National Academy of Sciences, 108(39), pp. 16469-16474; Nakamichi, N. (2015) ‘Adaptation to the Local Environment by Modifications of the Photoperiod Response in Crops’, Plant and Cell Physiology, 56(4), pp. 594-604; Zhang. B. et al. (2019) ‘Genetic Interactions Among Ghd7, Ghd8, OsPRR37 and Hd1 Contribute to Large Variation in Heading Date in Rice’, Rice, 12(1), p. 48). The Arabidopsis homolog of this gene is Pseudo Response Regulator 7 (PRR7; AT5G02810). Nakamichi, N. et at. (2007) ‘Arabidopsis Clock-Associated Pseudo-Response Regulators PRR9, PRR7 and PRR5 Coordinately and Positively Regulate Flowering Time Through the Canonical CONSTANS-Dependent Photoperiodic Pathway’, Plant and Cell Physiology, 48(6). pp. 822-832).

SbPRR37 is a central repressor in the sorghum flowering regulatory pathway that controls flowering in response to day length. Sorghum plants containing a non-functional, truncated version of SbPRR37 caused by early termination before the Response Regulatory domain flower independently of photoperiod (=autoflowering phenotype), whereas sorghum plants containing the full length functional version of SbPRR37 flowered in response to photoperiod (=photosensitive phenotype; Murphy. R. L. et al. (2011) ‘Coincident light and clock regulation of pseudoresponse regulator protein 37 (PRR37) controls photoperiodic flowering in sorghum’, Proceedings of the National Academy of Sciences, 108(39), pp. 18489-16474).

Alignment of sorghum (G4XFK1_SORBI Pseudo-response regulator 37) and Cannabis (XM_030632368.1_PREDICTED:_Cannabis_sativa_two-component_response_regulator-like_P RR37_(LOC115705128)_mRNA; CBDRx version CS10) PRR37 predicted amino acid sequences revealed that the Response Regulatory domain, which is located between amino acid positions 81-199 in SbPRR37 is located between amino acid positions 100-218 in CBDRx LOC115705128.

The PRR37 gene was subsequently further evaluated for variation in its coding sequence (CDS) in two autoflowering accessions and two photosensitive accessions (Table 6). RNA was extracted from leaf tissue collected two weeks after onset of flowering from these accessions (Table 6; Nucleospin RNA Plant and Fungi kit, Macherey-Nagel). Leaf tissue was used for this experiment because it is believed that signaling events resulting in flower formation take place in leaf (Zhang and Chen 2021; PLoS Biology 19.2 (2021): e3001099).

After concentration adjustment and treatment with DNAse the RNA was used directly for RT-PCR (OneTaq® One-Step RT-PCR Kit, New England Biolabs). Sanger sequencing was performed based on cloned RT-PCR product (NEB PCR® Cloning Kit; New England Biolabs) for areas with high levels of heterozygosity, Sanger sequencing based on PCR product was performed for areas with low levels of heterozygosity. Genomic DNA (extracted from leaf tissue with a NucleoMag Plant DNA Kit, Macherey-Nagel) was used to sequence the beginning of the gene as well as part of the Response Regulatory domain. Primers used for amplification and sequencing of fragments of PRR37 can be found in Table 8.

TABLE 8

| | | Start | End | |
|---|---|---|---|---|
| SEQ ID NO | Description | position | position | Sequence |
| SEQ ID | PRR37S_1AF | −244 | −223 | GGGACTTTGATCAGAACACACA |
| NO: 198 | | | | |
| SEQ ID | PRR37S_1AR | 389 | 369 | GCTGAAACGTACTGACCTTCG |
| NO: 199 | | | | |
| SEQ ID | PRR37C_1BF | 27 | 46 | AGCTCCCGTAACCAATGATG |
| NO: 200 | | | | |
| SEQ ID | PRR37C_7AR | 1086 | 1067 | CATGTTGGTCAGCCCTTTTT |
| NO: 201 | | | | |
| SEQ ID | PRR37C_7BR* | 1225 | 1206 | TGGAGACACCCATCAGATCA |
| NO: 202 | | | | |
| SEQ ID | PRR37K_A_F | 1557 | 1576 | GGCAGACAAGCTTGGAAAGT |
| NO: 203 | | | | |
| SEQ ID | PRR37K_A_R | 2310 | 2291 | CTCATCATTGCTGCCACTGT |
| NO: 204 | | | | |
| SEQ ID | PRR37K_B_R* | 1951 | 1932 | TGTGGCATTTTCTCCAAACA |
| NO: 205 | | | | |
| SEQ ID | AF1 PRR37 | 1 | 1090 | ATGAAAATGGTCCAGATGAATAATAAAGCTCCCGTAAC |
| NO: 206 | CDS splice | | | CAATGATGAGCTAACTGAGCTGAATCACCGGATTCAA |
| | variant X1 | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGGTAGTCATGCCCTGTTTATCTGG |
| | | | | TATTGGTCTTCTAGGCAAGATCATGAGCAAAAAAACAT |
| | | | | GCAAGGACATCCCTGTAATTATTGATGATGTCTTCACA |
| | | | | TGATTCTAGGAGTATGGTCTTTAAGTGTTTATCGAAAG |
| | | | | GTGCCGTTGACTTTTTAGTGAAACCTATTCGAAAGAA |
| | | | | TGAGCTGAAAAACCTTTGGCAACATGTTTGGAGAAAA |
| | | | | TGCCACATTTCTAGTAATAGTGGAAGTGAAAGTGGTAT |
| | | | | ATGGATTGAAAAGCCTTTAAAGTCAAGAGCTGTGGAA |
| | | | | CATTCAGACAACAACAGTGGCAGCAATGATGAGGAT |
| | | | | GATACTGACAGCATTGGTCTAAATTTCAGGAATGAAA |
| | | | | GTGACAGTGGAACACAGAGCTCTTGGACAAAGAGAG |
| | | | | CAGAAGTTGACAGCCCTCAGGCAGTGTCGTGGGAG |
| | | | | CAGTTTGCTGATCTTCCTGATAGCACTAATCATCAGGT |
| | | | | CAATCATCCAAGGCAAGAAGCCTTTGGAAACAACTG |
| | | | | GGTACCTGAAAATGCAACAGTAACACCACGCCCACAT |
| | | | | AATGATGAGCTTGACAAAAAAGTCATGGGAAAAGACT |
| | | | | TGAAAATAGGATTACCTAGCCTTCTTGAAGACACAAG |
| | | | | TGAAAAAGGGCTGACCAACATG |
| SEQ ID | AF1 PRR37 | 1 | 1004 | ATGAAAATGGTCCAGATGAATAATAAAGCTCCCGTAAC |
| NO: 207 | CDS splice | | | CAATGATGAGCTAACTGAGCTGAATCACCGGATTCAA |
| | variant X2 | | | GATGGGAAAAAAGAAAGAAGGGAGAGGGTTACGAGA |
| | | | | GAACGCCAAGGGCTCTCAGAGGAACATGAATCTTGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTRGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGTGATGTCTTCACATGATTCTAGG |
| | | | | AGTATGGTCTTTAAGTGTTTATCGAAAGGTGCCGTTG |
| | | | | ACTTTTTAGTGAAACCTATTCGAAAGAATGAGCTGAAA |
| | | | | AACCTTTGGCAACATGTTTGGAGAAAATGCCACATTT |
| | | | | CTAGTAATAGTGGAAGTGAAAGTGGTATATGGATTGAA |
| | | | | AAGCCTTTAAAGTCAAGAACTGTGGAACATTCAGACA |
| | | | | ACAACAGTGGCAGCAATGATGAGGATGATACTGACA |
| | | | | GCATTGGTCTAAATTTCAGGAATGAAAGTGACAGTGG |
| | | | | AACACAGAGCTCTTGGACAAAGAGAGCAGAAGTTGA |
| | | | | CAGCCCTCAGGCAGTGTCGTGGGAGCAGTTTGCTGA |
| | | | | TCTTCCTGATAGCACTAATCATCAGGTCAATCATCCAA |
| | | | | GGCAAGAAGCCTTTGGAAACAACTGGGTACCTGAAA |
| | | | | ATGCAACAGTAACACCACGCCCACATAATGATGAGCT |
| | | | | TGACAAAAAAGTCATGGGAAAAGACTTGAAAATAGGA |
| | | | | TTACCTAGCCTTCTTGAAGACACAAGTGAAAAAGGGC |
| | | | | TGACCAACATG |
| SEQ ID | AF2 PRR37 | 1 | 1089 | ATGAAAATGGTCCAGATGAATAATAAAGCTCCCGTAAC |
| NO: 208 | CDS splice | | | CAATGATGAGCTAACTGAGCTGAATCACCGGATTCAA |
| | variant X1 | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGGTAGTCATGCCCTGTTTATCTGG |
| | | | | TATTGGTCTTCTAGGCAAGATCATGAGCAAAAAAACAT |
| | | | | GCAAGGACATCCCTGTAATTATTGATGATGTCTTCACA |
| | | | | TGATTCTAGGAGTATGGTCTTTAAGTGTTTATCGAAAG |
| | | | | GTGCCGTTGACTTTTTAGTGAAACCTATTCGAAAGAA |
| | | | | TGAGCTGAAAAACCTTTGGCAACATGTTTGGAGAAAA |
| | | | | TGCCACATTTCTAGTAATAGTGGAAGTGAAAGTGGTAT |
| | | | | ATGGATTGAAAAGCCTTTAAAGTCAAGAACTGTGGAA |
| | | | | CATTCAGACAACAACAGTGGCAGCAATGATGAGGAT |
| | | | | GATACTGACAGCATTGGTCTAAATTTCAGGAATGAAA |
| | | | | GTGACAGTGGAACACAGAGCTCTTGGACAAAGAGAG |
| | | | | CAGAAGTTGACAGCCCTCAGGCAGTGTCGTGGGAG |
| | | | | CAGTTTGCTGATCTTCCTGATAGCACTAATCATCAGGT |
| | | | | CAACCATCCAAGGCAAGAAGCCTTTGGAAACAACTG |
| | | | | GGTACCTGAAAATGCAACAGTAACACCACGCCCACAT |
| | | | | AATGATGAGCTTGACAAAAAAGTCATGGGAAAAGACT |
| | | | | TGAAAATAGGATTACCTAGCCTTCTTGAGGACACAAG |
| | | | | TGAAAAAGGGCTGACCAACAT |
| SEQ ID | AF2 PRR37 | 1 | 851 | ATGAAAATGGTCCAGATGAATAATAAAGCTCCCGTAAC |
| NO: 209 | CDS splice | | | CAATGATGAGCTAACTGAGCTGAATCACCGGATTCAA |
| | variant X2 | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGTGATGTCTTCACATGATTCTAGG |
| | | | | AGTATGGTCTTTAAGTGTTTATCGAAAGGTGCCGTTG |
| | | | | ACTTTTTAGTGAAACCTATTCGAAAGAATGAGCTGAAA |
| | | | | AACCTTTGGCAACATGTTTGGAGAAAATGCCACATTT |
| | | | | CTAGTAATAGTGGAAGTGAAAGTGGTATATGGATTGAA |
| | | | | AAGCCTTTAAAGTCAAGAACTGTGGAACATTCAGACA |
| | | | | ACAACAGTGGCAGCAATGATGAGGATGATACTGACA |
| | | | | GCATTGGTCTAAATTTCAGGAATGAAAGTGACAGTGG |
| | | | | AACACAGAGCTCTTGGACAAAGAGAGCAGAAGTTGA |
| | | | | CAGCCCTCAGGCAGTGTCGTGGGAGCAGTTTGCTGA |
| | | | | TCTTCCTGATAGCACTAATCATCAGGTCAATCATCCAA |
| | | | | GGCAA |
| SEQ ID | AF2 PRR37 | 1 | 1079 | ATGAAAATGGTCCAGATGAATAATAAAGCTCCCGTAAC |
| NO: 210 | CDS splice | | | CAATGATGAGCTAACTGAGCTGAATCACCGGATTCAA |
| | variant X3 | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGGTAGTCATGCCCTGTTTATCTGG |
| | | | | TATTGGTCTTCTAGGCAAGATCATGAGCAAAAAAACAT |
| | | | | GCAAGGACATCCCTTGATGTCTTCACATGATTCTAGG |
| | | | | AGTATGGTCTTTAAGTGTTTATCGAAAGGTGCCGTTG |
| | | | | ACTTTTTAGTGAAACCTATTCGAAAGAATGAGCTGAAA |
| | | | | AACCTTTGGCAACATGTTTGGAGAAAATGCCACATTT |
| | | | | CTAGTAATAGTGGAAGTGAAAGTGGTATATGGATTGAA |
| | | | | AAGCCTTTAAAGTCAAGAACTGTGGAACATTCAGACA |
| | | | | ACAACAGTGGCAGCAATGATGAGGATGATACTGACA |
| | | | | GCATTGGTCTAAATTTCAGGAATGAAAGTGAGAGTGG |
| | | | | AACACAGAGCTCTTGGACAAAGAGAGCAGAAGTTGA |
| | | | | CAGCCCTCAGGCAGTGTCGTGGGAGCAGTTTGCTGA |
| | | | | TCTTCCTGATAGCACTAATCATCAGGTCAATCATCCAA |
| | | | | GGCAAGAAGCCTTTGGAAACAACTGGGTACCTGAAA |
| | | | | ATGCAACAGTAACACCACGCCCACATAATGATGAGCT |
| | | | | TGACAAAAAAGTCATGGGAAAAGACTTGAAAATAGGA |
| | | | | TTACCTAGCCTTCTTGAGGACACAAGTGAAAAAGGG |
| | | | | CTGACCAACATG |
| SEQ ID | PS1 PRR37 | 1 | 2172 | ATGAAAATGGTCCAGATGAATAATAAAGCTCCCGTAAC |
| NO: 211 | CDS*** | | | CAATGATGAGCTAACTGAGCTGAATCACCGGATTCAA |
| | | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGGTAGTCATGCCGTGTTTATCTGG |
| | | | | TATTGGTCTTCTAGGCAAGATCATGAGCAAAAAAACAT |
| | | | | GCAAGGACATCCCTGTAATTATGATGTCTTCACATGAT |
| | | | | TCTAGGAGTATGGTCTTTAAGTGTTTATCGAAAGGTG |
| | | | | CCGTTGACTTTTTAGTGAAACCTATTCGAAAGAATGA |
| | | | | GCTGAAAAACCTTTGGCAACATGTTTGGAGAAAATGC |
| | | | | CACATTTCTAGTAATAGTGGAAGTGAAAGTGGTATATG |
| | | | | GATTGAAAAGCCTTTAAAGTCAAGAACTGTGGAACAT |
| | | | | TCAGACAACAACAGTGGCAGCAATGATGAGGATGATA |
| | | | | CTGACAGCATTGGTCTAAATTTCAGGAATGAAAGTGA |
| | | | | CAGTGGAACACAGAGCTCTTGGACAAAGAGAGCAGA |
| | | | | AGTTGACAGCCCTCAGGCAGTGTCGTGGGAGCAGTT |
| | | | | TGCTGATCTTCCTGATAGCACTAATCATCAGGTCAATC |
| | | | | ATCCAAGGCAAGAAGCCTTTGGAAACAACTGGGTAC |
| | | | | CTGAAAATGCAACAGTAACACCACGCCCACATAATGA |
| | | | | TGAGCTTGACAAAAAAGTCATGGGAAAAGACTTGAAA |
| | | | | ATAGGATTACCTAGCCTTCTTGAAGACACAAGTGAAA |
| | | | | AAGGGCTGACCAACATGGAAGGTACTAATAAAGATAA |
| | | | | ATGTTCTGAACTGAACTCAAAGAAAGATGATCAGGAG |
| | | | | CAGGAGAAAAGGGAATTAGACCTCAACAATGAAGAA |
| | | | | CCGAGTGCAGAAAAGACCCAAGCTGTTGATCTGATG |
| | | | | GGTGTCTCCAATTATAGTATTGATCCTCACATGGAAAG |
| | | | | TGGAGTTCTTGATGTCCCAAACGAACTCTCCAAGGCT |
| | | | | GCCTGCATGAGAGATAATGCCAACCATGAGAATAAAG |
| | | | | AAACACCTTTTTTTGAGCTCATTTTAAAGAGGCCAAG |
| | | | | AGATATCCAAGATACTGGAACCAGCGCACACGATCGA |
| | | | | AATGTTTTGAGACATTCCGATATTTCAGCTTTTTCAAG |
| | | | | GTATAACATTGTTTCAACTGCAAATCAAGCTCCAACAG |
| | | | | GGAACATAGGGAGCTGTTCTCCTCTAGATAATAGCTC |
| | | | | AGATGCAGCAAAAACAGAATCAATCCCAAATTTGCAA |
| | | | | TCTGATTCAAATGGTACACCTCCCAACCAGGGTTCCA |
| | | | | ATGGTAGTAGCAACAATAATGACATGGGATCCACTAC |
| | | | | GAATAATGCTTTTACCAAACAAGTGGCTTTTGCAGAG |
| | | | | AGGCCTACAAACAAATCCACAATCAAACTCCAATCAA |
| | | | | ACACTGGTTTCCAACAAGTGCAAAATGGCCAAGCTTC |
| | | | | CCTTCAGACTATTATTCAAGATGCTTCACAGTGTGGTT |
| | | | | CATCCAATGCATTGAGAGCACCCATGGAAGGCAATAT |
| | | | | TAGTAATCACAGTCTCAACAGGAGTGGGTCAGGTAGT |
| | | | | AACCATGGTAGCAACGGACAAAAGAGAAGCACCAAT |
| | | | | GCTTCAAACTCCAGAGGGAAAAAGACAGAAAGTGAG |
| | | | | AGTGTGGTTACTGGAAGAGGGAAAACCATTGAAGGA |
| | | | | AGTGAATCGGATGGAAATCGATTTGCACAAAGAGAAG |
| | | | | CTGCTTTGAAAAGATTCCGCCAGAAGAGGCAAGAAA |
| | | | | GATGCTTTGAGAAAAAGGTGAGATATCAGAGTAGAAA |
| | | | | AAAACTGGCAGAACAAAGACCCCGAATTCGAGGACA |
| | | | | ATTTATTAGAAAGGGAATGAATGAAAACAAGGGAAAA |
| | | | | GGCATAAATTACGAACCTGAACCAATTTCATAA |
| SEQ ID | PS2 PRR37 | 1 | 1182 | ATGAAAATGGTCCAGATGAATAATAAAGCTCCCGTAAC |
| NO: 212 | CDS | | | CAATGATGAGCTAACTGAGCTGAATCACCGGATTCAA |
| | | | | GATGGGAAAAAAGAAAGAAGGGAGAGGGTTACGAGA |
| | | | | GAACGCCAAGGGCTCTCAGAGGAACATGAATCTAGG |
| | | | | ATCAATGARGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGCGCGCTACTAAGAAATTGTGG |
| | | | | CTACGAAGTTACAGCTGTAGAAAATGGCAGACAAGCT |
| | | | | TGGAAAGTCTTAGAAGATCTTGTGACAGATGTTGATC |
| | | | | TCGTTTTGACTGAGGTAGTCATGCCCTGTTTATCTGG |
| | | | | TATTGGTCTTCTAGGCAAGATCATGAGCAAAAAAACAT |
| | | | | GCAAGGACATCCCTGTAATTATGATGTCTTCTCATGAT |
| | | | | TCTAGGAGTATGGTCTTTAAGTGTTTATCGAAAGGTG |
| | | | | CCGTTGACTTTTTAGTGAAACCTATTCGAAAGAATGA |
| | | | | GCTGAAAAACCTTTGGCAACATGTTTGGAGAAAATGC |
| | | | | CACATTTCTAGTAATAGTGGAAGTGAAAGTGGWATAT |
| | | | | GGATTGAAAAGCCTTTAAAGTCAAGAACTGTGGAACA |
| | | | | TTCWGACAACAACAGTGGCAGCAATGATGAGGATGA |
| | | | | YACTGACAGCATTGGTCTAAATTTCAGGAATGAAAGT |
| | | | | GACAGTGGAACACAGAGCTCTTGGACAAAGAGAGCA |
| | | | | GAAGTTGACAGCCCTCAGGCAGTGTCGTGGGAGCA |
| | | | | GTTTGCTGATCTTCCTGATAGCACTAATCATCAGGTCA |
| | | | | ATCATCCAAGGCAAGAAGCCTTTGGAAACAACTGGG |
| | | | | TACCTGAAAATGCAACAGTAACACCACGCCCACATAA |
| | | | | TGATGAGCTTGACAAAAAAGTCATGGGAAAAGACTTG |
| | | | | AAAATAGGATTACCTAGCCTTCTTGAAGACACAAGTG |
| | | | | AAAAAGGGCTGACCAACATGGAAGGTACTAATAAAGA |
| | | | | TAAATGTTCTGAACTGAACTCAAAGAAAGATGATCAG |
| | | | | GAGCAGGAGAAAAGGGAATTAGACCTCAACAATGAA |
| | | | | GAACCG |
| SEQ ID | Predicted | 1 | 184 | MKMVQMNNKAPVTNDELTELNHRIQDGKKERRERVTR |
| NO: 213 | Cannabis | | | ERQGLSEEHESRINEDVQQHVSN |
| | protein | | | GEIGTVQALERSHSGQRRSQQQPQGHLVRWERFLAF |
| | sequence for | | | RSLKVLLVENDDSTRHIVSALLRN |
| | AF1 PRR37 | | | CGYEVTAVENGRQAWKVLEDLVTCVCLVLTEVVMPCLS |
| | splice variant | | | GIGLLGKIMSKKTCKDIPVIID |
| | X1 | | | DVFT* |
| SEQ ID | Predicted | 1 | 151 | MKMVQMNNKAPVINDELTELNHRIQDGKKERRERVTR |
| NO: 214 | Cannabis | | | ERQGLSEEHESWINEDVQQHVSN |
| | protein | | | GEIGTVQALERSHSGQRRSQQQPQGHLVRWERFLAF |
| | sequence for | | | RSLKVLLVENDDSTRHIVSALLRN |
| | AF1 PRR37 | | | CGYEVTAVENGRCAWKVLEDLVTDVDLVLTE* |
| | splice variant | | | |
| | X2 | | | |
| SEQ ID | Predicted | 1 | 184 | MKMVQMNNKAPVINDELTELNHRIQDGKKERRERVTR |
| NO: 215 | Cannabis | | | ERQGLSEEHESRINEDVQQHVSN |
| | protein | | | GEIGTVQALERSHSGQRRSQQQPQGHLVRWERFLAF |
| | sequence for | | | RSLKVLLVENDDSTRHIVSALLRN |
| | AF2 PRR37 | | | CGYEVTAVENGRQAWKVLEDLVTCVCLVLTEVVMPCLS |
| | splice variant | | | GIGLLGKIMSKKTCKDIPVIID |
| | X1 | | | DVFT* |
| SEQ ID | Predicted | 1 | 151 | MKMVQMNNKAPVTNDELTELNHRIQDGKKERRERVTR |
| NO: 216 | Cannabis | | | ERQGLSEEHESRINEDVQQHVSN |
| | protein | | | GEIGTVQALERSHSGQRRSQQQPQGHLVRWERFLAF |
| | sequence for | | | RSLKVLLVENDDSTRHIVSALLRN |
| | AF2 PRR37 | | | CGYEVTAVENGRQAWKVLEDLVTDVDLVLTE* |
| | splice variant | | | |
| | X2 | | | |
| SEQ ID | Predicted | 1 | 176 | MKMVQMNNKAPVTNDELTELNHRIQDGKKERRERVTR |
| NO: 217 | Cannabis | | | ERQGLSEEHESRINEDVQQHVSN |
| | protein | | | GEIGTVQALERSHSGQRRSQQQPQGHLVRWERFLAF |
| | sequence for | | | RSLKVLLVENDDSTRHIVSALLRN |
| | AF2 PRR37 | | | CGYEVTAVENGRQAWKVLEDLVTDVDLVLTEVVMPCLS |
| | splice variant | | | GIGLLGKIMSKKTCKDIP* |
| | X3 | | | |
| SEQ ID | Predicted | 1 | 723 | MKMVQMNNKAPVINDELTELNHRIQDGKKERRERVTR |
| NO: 218 | Cannabis | | | ERQGLSEEHESRINEDVQQHVSNGEIGTVQALERSHS |
| | protein | | | GQRRSQQQPQGHLVRWERFLAFRSLKVLLVENDDSTR |
| | sequence for | | | HIVSALLRNCGYEVTAVENGRCAWKVLEDLVTDVDLVLT |
| | PS1 PRR37*** | | | EVVMPCLSGIGLLGKIMSKKTCKDIPVIMMSSHDSRSM |
| | | | | VFKCLSKGAVDFLVKPIRKNELKNLWQHVWRKCHISSN |
| | | | | SGSESGIWIEKPLKSRTVEHSDNNSGSNDEDDTDSIGL |
| | | | | NFRNESDSGTQSSWTKRAEVDSPQAVSWEQFADLPD |
| | | | | STNHQVNHPRQEAFGNNWVPENATVTPRPHNDELDK |
| | | | | KVMGKDLKIGLPSLLEDTSEKGLTNMEGTNKDKCSELN |
| | | | | SKKDDQEQEKRELDLNNEEPSAEKTQAVDLMGVSNYS |
| | | | | IDPHMESGVLDVPNELSKAACMRDNANHENKETPFFE |
| | | | | LILKRPRDIQDTGTSAHDRNVLRHSDISAFSRYNIVSTAN |
| | | | | QAPTGNIGSCSPLDNSSDAAKTESIPNLQSDSNGTPPN |
| | | | | QGSNGSSNNNDMGSTTNNAFTKQVAFAERPTNKSTIK |
| | | | | LQSNTGFQQVCNGQASLQTIIQDASQCGSSNALRAPM |
| | | | | EGNISNHSLNRSGSGSNHGSNGQKRSTNASNSRGKK |
| | | | | TESESVVTGRGKTIEGSESDGNRFAQREAALKRFRCK |
| | | | | RQERCFEKKVRYQSRKKLAEQRPRIRGQFIRKGMNEN |
| | | | | KGKGINYEPEPIS* |
| SEQ ID | Predicted | 1 | 394 (of | MKMVQMNNKAPVTNDELTELNHRIQDGKKERRERVTR |
| NO: 219 | Cannabis | | 723)** | ERQGLSEEHESRINEDVQQHVSN |
| | protein | | | GEIGTVQALERSHSGQRRSQQQPQGHLVRWERFLAF |
| | sequence for | | | RSLKVLLVENDDSTRHIVSALLRN |
| | PS2 PRR37 | | | CGYEVTAVENGRQAWKVLEDLVTDVDLVLTEVVMPCLS |
| | | | | GIGLLGKIMSKKTCKDIPVIMM |
| | | | | SSHDSRSMVFKCLSKGAVDFLVKPIRKNELKNLWQHV |
| | | | | WRKCHISSNSGSESGIWIEKPLK |
| | | | | SRTVEHSDNNSGSNDEDDTDSIGLNFRNESDSGTQSS |
| | | | | WTKRAEVDSPQAVSWEQFADLPD |
| | | | | STNHQVNHPRQEAFGNNWWVPENATVTPRPHNDELDK |
| | | | | KVMGKDLKIGLPSLLEDTSEKGLT |
| | | | | NMEGTNKDKCSELNSKKDDQEQEKRELDLNNEEP |
| SEQ ID | AF1 PRR37 | 1557 | 2310 | GGCAGACAAGCTTGGAAAGTCTTAGAAGATCTTGTG |
| NO: 220 | Response | | | ACAGATGTTGATCTCGTTTTGACTGAGGTAGTCATGC |
| | Regulatory | | | CCTGTTTATCTGGTATTGGTCTTCTAGGCAAGATCATG |
| | domain | | | AGCAAAAAARCATGCAAGGACATCCCTGTAATTATTG |
| | genomic DNA | | | AGTATATTTCCTTTATTTTGGATTTAAAATAATACTTTTT |
| | | | | TCTCTAGTATCTTTTTGTAATTTATACTTTTAACTAATAC |
| | | | | ATTTATTGTGTGTGTTTGTGTTTGTGTTTTCACAGTGA |
| | | | | TGTCTTCACATGATTCTAGGAGTATGGTCTTTAAGTGT |
| | | | | TTATCGAAAGGTGCCGTTGACTTTTTAGTGAAACCTAT |
| | | | | TCGAAAGAATGAGCTGAAAAACCTTTGGCAACATGTT |
| | | | | TGGAGAAAATGCCACATTGTGAGTTGCAATCTATTTG |
| | | | | ATTTATTATATCATGGACCAATTCACCTTGAGGTTCAG |
| | | | | GTTTCTTCTCATTTATGCATTTTGTTTTTCATTAAGCAC |
| | | | | CAATTGTCGAACTATACCAAAGAAAACAAACAAAAAAA |
| | | | | TGGCTTCAATCTTTGCTTGATAAAAAATATGTTTTATGT |
| | | | | AATGCAAACATCACTTAATTGACATAATACCATTAAACA |
| | | | | CAGAAATTTGCTCGAGTTGTGCATTAATTTTTCATATTT |
| | | | | TCATCAGTCTAGTAATAGTGGAAGTGAAAGTGGTATAT |
| | | | | GGATTGAAAAGCCTTTAAAGTCAAGAACTGTGGAACA |
| | | | | TTCAGACAACAACAGTGGCAGCAATGATGAG |
| SEQ ID | AF2 PRR37 | 1598 | 1951 | TGTTGATCTCGTTTTGACTGAGGTAGTCATGCCCTGT |
| NO: 221 | Response | | | TTATCTGGTATTGGTCTTCTAGGCAAGATCATGAGCAA |
| | Regulatory | | | AAAAACATGCAAGGACATCCCTGTAATTATTGAGTATA |
| | domain | | | TTTCCTTTATTTTGGATTTAAAATAATACTTTTTTCTCTA |
| | genomic DNA | | | GTATCTTTTTGTAATTTATACTTTTAACTAATACATTTATT |
| | | | | GTGTGTGTTTGTGTTTGTGTTTTCACAGTGATGTCTT |
| | | | | CACATGATTCTAGGAGTATGGTCTTTAAGTGTTTATCG |
| | | | | AAAGGTGCCGTTGACTTTTTAGTGAAACCTATTCGAA |
| | | | | AGAATGAGCTGAAAAACCTTTGGCAACATGTTTGGAG |
| | | | | AAAATGCCACA |
| SEQ ID | PS1 PRR37 | 1599 | 1938 | GTTGATCTCGTTTTGACTGAGGTAGTCATGCCCTGTT |
| NO: 222 | Response | | | TATCTGGTATTGGTCTTCTAGGCAAGATCATGAGCAAA |
| | Regulatory | | | AAAACATGCAAGGACATCCCTGTAATTAGTGAGTATAT |
| | domain | | | TTCCTTTATTTTGGATTTAAAATAATACTTTTTTCTCTAG |
| | genomic DNA | | | TATCTTTTTGTAATTTATACTTTTAACTAATACATTTATTG |
| | | | | TGTGTGTTTGTGTTTGTGTTTTCACAGTGATGTCTTCT |
| | | | | CATGATTCTAGGAGTATGGTCTTTAAGTGTTTATCGAA |
| | | | | AGGTGCCGTTGACTTTTTAGTGAAACCTATTCGAAAG |
| | | | | AATGAGCTGAAAAACCTTTGGCAACATGTTTGG |
| SEQ ID | PS2 PRR37 | 1598 | 1943 | TGTTGATCTCGTTTTGACTGAGGTAGTCATGCCCTGT |
| NO: 223 | Response | | | TTATCTGGTATTGGTCTTCTAGGCAAGATCATGAGCAA |
| | Regulatory | | | AAAAACATGCAAGGACATCCCTGTAATTAGTGAGTATA |
| | domain | | | TTTCCTTTATTTTGGATTTAAAATAATACTTTTTTCTCTA |
| | genomic DNA | | | GTATCTTTTTGTAATTTATACTTTTAACTAATACATTTATT |
| | | | | GTGTGTGTTTGTGTTTGTGTTTTCACAGTGATGTCTT |
| | | | | CTCATGATTCTAGGAGTATGGTCTTTAAGTGTTTATCG |
| | | | | AAAGGTGCCGTTGACTTTTTAGTGAAACCTATTCGAA |
| | | | | AGAATGAGCTGAAAAACCTTTGGCAACATGTTTGGAG |
| | | | | AAA |
| SEQ ID | PS1 PRR37 | 1 | 4630 | ATGAAAATGGTCCAGATGAATAATAAAGCTCCCGTAAC |
| NO: 224 | genomic | | | CAATGATGAGCTAACTGAGCTGAATCACCGGATTCAA |
| | DNA*** | | | GATGGGAAAAAAGAAAGAAGGGAGAGGGTTACGAGA |
| | | | | GAACGCCAAGGGCTCTCAGAGGAACATGAATCTAGG |
| | | | | ATCAATGAAGATGTGCAACAACATGTCAGCAATGGGG |
| | | | | AGATTGGAACAGTACAGGCTCTGGAGAGGAGTCATT |
| | | | | CTGGCCAGCGGAGGTCTCAGCAACAGCCTCAAGGA |
| | | | | CATTTGGTTCGCTGGGAGAGGTTCCTAGCTTTCAGG |
| | | | | TCGCTAAAGGTTTTATTGGTGGAAAATGATGACTCAA |
| | | | | CTCGCCATATTGTCAGGGGGCTACTAAGAAATTGTGG |
| | | | | CTACGAAGGTCAGTACGTTTCAGCAACATGAAATGAT |
| | | | | CATTGCATTTCTCTTATGCAGAAGTATACACTCTATGTT |
| | | | | AATGGCTTTATGTTTGATTATTGACGGAGATGACTTTT |
| | | | | AAAGTAAACACTGTAGTAGATCTTATGAATAAACTTGA |
| | | | | TGTTATATGATATGAAAGGAAACCATGTTTATTTGCTCC |
| | | | | ACTACGAGTAATAATATCCATGGTCTAATAGTTGTTGT |
| | | | | CACTAACCATAACCTAGAAAGAGTGGTCTTAGGATTA |
| | | | | GCCTCGCTGATCCACACCTCACCTCTTGTAGAGAAG |
| | | | | GTCAAGGGTTCAATCCCTCCCCCGCCCTCCAAAGAA |
| | | | | AGATATAACAAACAGAAAACGAATCAAAAGAATAAAAA |
| | | | | ACAAACAAGTAAGGTGAGCATGGCACTATTATTAGAC |
| | | | | TAAAGCTTGTTTTAGTTTAATATAAGTAGATCTTATTTCT |
| | | | | GAAATGTTATTTTCCACCATCTAATTACACACATTTCTA |
| | | | | GACTGATCCCCTGATCTTGTGGCAGCGCTCTTTTTCT |
| | | | | GAGTTCCTATCAGAAATGCTGAAAACTATTAAACTAGT |
| | | | | TTTTGTTACTTATTTATTTTCTTTTGTTTTAACACTACAT |
| | | | | TTTAACAACAGTTCCTATACTCTGGTGCTTCACGTGTC |
| | | | | TATTTGTGCTATTTTGATGTTCATATTTATAGTCTAGCG |
| | | | | GGAAGTTTTTTAGTCATTTCGTTCATGAAGGGTCAAG |
| | | | | TACGATTTCTTGACCTAGCTTAGATTTTGACATAGAAC |
| | | | | CATTCTTGAGGATACTACAGTGGGTTACTTAGTTTGTA |
| | | | | GAGTATGTTTATGTGTACCTTCTAAAGATAACCTGGTT |
| | | | | ATAAGTATGATATTCTCGAAAAGAAAAACATGTTGCTC |
| | | | | CAGACCTGTTGGCAATTGACATCTTACTTTCTAGCTAT |
| | | | | GATAATTAGACATTCAGTCGCTATATTTATGTCATGCTT |
| | | | | TCTTCATCATCATTTTTCATATATGTGTTCAAGTTATGG |
| | | | | TAAGCTATTTAAATATTGTTATTTTATTTGACCATGTTTT |
| | | | | ATTTCCAACGCAAACTGCTTGGTATAATATTACATAAAT |
| | | | | GTTAGCCAGAAGCTTTGATTTAGTTTACATCCTAAAAG |
| | | | | TCAAAACTAGAATTTTATTGGTTCCCTCAATAAACTAA |
| | | | | CAATTATTCAATGTATTCTCATATTGGCAGTTACAGCTG |
| | | | | TAGAAAATGGCAGACAAGCTTGGAAAGTCTTAGAAGA |
| | | | | TCTTGTGACAGATGTTGATCTCGTTTTGACTGAGGTA |
| | | | | GTCATGCCGTGTTTATCTGGTATTGGTCTTCTAGGCAA |
| | | | | GATCATGAGCAAAAAAACATGCAAGGACATCCCTGTA |
| | | | | ATTAGTGAGTATATTTCCTTTATTTTGGATTTAAAATAAT |
| | | | | ACTTTTTTCTCTAGTATCTTTTTGTAATTTATACTTTTAA |
| | | | | CTAATACATTTATTGTGTGTGTTTGTGTTTGTGTTTTCA |
| | | | | CAGTGATGTCTTCACATGATTCTAGGAGTATGGTCTTT |
| | | | | AAGTGTTTATCGAAAGGTGCCGTTGACTTTTTAGTGA |
| | | | | AACCTATTCGAAAGAATGAGCTGAAAAACCTTTGGCA |
| | | | | ACATGTTTGGAGAAAATGCCACATTGTGAGTTGCAAT |
| | | | | CTATTTGATTTATTATATCATGGACCAATTCACCTTGAG |
| | | | | GTTCAGGTTTCTTCTCATTTATGCATTTTGTTTTTCATT |
| | | | | AAGCACCAATTGTCGAACTATACCAAAGAAAACAAAC |
| | | | | AAAAAAATGGCTTCAATCTTTGCTTGATAAAAAATATG |
| | | | | TTTTATGTAATGCAAACATCACTTAATTGACATAATACC |
| | | | | ATTAAACACAGAAATTTGCTCGAGTTGTGCATTAATTT |
| | | | | TTCATATTTTCATCAGTCTAGTAATAGTGGAAGTGAAA |
| | | | | GTGGTATATGGATTGAAAAGCCTTTAAAGTCAAGAAC |
| | | | | TGTGGAACATTCAGACAACAACAGTGGCAGCAATGAT |
| | | | | GAGGATGATACTGACAGCATTGGTCTAAATTTCAGGA |
| | | | | ATGAAAGTGACAGTGGAACACAGGTATTTCACTAAAT |
| | | | | TTCATGAAAGAGTTTTGTTTTTTTTTTTTGGTGGGAAT |
| | | | | GAAGTTTTATGTCTTTGTATTTACAGAAATATTAGCGAA |
| | | | | ATGTTAGTTTCCACATGAAGTTTTATGTTTTATGTATCA |
| | | | | AGAAACTACTATAATTATGTTAGTTTCCACACACCTAGT |
| | | | | TTGGAATTTGTCTATCACAGCACCCATATATCTATTTAC |
| | | | | CATGTTTGCATATTACTCATTTGATCTTGGTGGAGGAA |
| | | | | TATTCATGTAAGAGTTTTTAATACTTTTATGTATATGTAA |
| | | | | GTGGAGAAGGAATGATAATTAGCAAGATAAGAAAACA |
| | | | | AGAAAAAGAATGAAAACTTACTACTGAGCTTTTACAGA |
| | | | | GCTCTTGGACAAAGAGAGCAGAAGTTGACAGCCCTC |
| | | | | AGGCAGTGTCGTGGGAGCAGTTTGCTGATCTTCCTG |
| | | | | ATAGCACTAATCATCAGGTCAATCATCCAAGGCAAGA |
| | | | | AGCCTTTGGAAACAACTGGGTACCTGAAAATGCAACA |
| | | | | GTAACACCACGCCCACATAATGATGAGCTTGGTAGGC |
| | | | | AAATTTTCCTAACATCTTTTTTTTTTTTAACTTTCTTCC |
| | | | | ATTCTCGATCATTCTCTAAGTTATGATTTATTTAAATTGT |
| | | | | TTAGACAAAAAAGTCATGGGAAAAGACTTGAAAATAG |
| | | | | GATTACCTAGCCTTCTTGAAGACACAAGTGAAAAAGG |
| | | | | GCTGACCAACATGGAAGGTACTAATAAAGATAAATGTT |
| | | | | CTGAACTGAACTCAAAGAAAGATGATCAGGAGCAGG |
| | | | | AGAAAAGGGAATTAGACCTCAACAATGAAGAACCGA |
| | | | | GTGCAGAAAAGACCCAAGCTGTTGATCTGATGGGTG |
| | | | | TCTCCAATTATAGTATTGATCCTCACATGGAAAGTGGA |
| | | | | GTTCTTGATGTCCCAAACGAACTCTCCAAGGCTGCCT |
| | | | | GCATGAGAGATAATGCCAACCATGAGAATAAAGAAAC |
| | | | | ACCTTTTTTTGAGCTCATTTTAAAGAGGCCAAGAGATA |
| | | | | TCCAAGATACTGGAACCAGCGCACACGATCGAAATGT |
| | | | | TTTGAGACATTCCGATATTTCAGCTTTTTCAAGGTATA |
| | | | | GAAATATTTTGTGTTGATATAAACATGCTCAATAACAAT |
| | | | | TAACTCTAAATTTAAGAATATAACTAAGTCCCATTACCA |
| | | | | TGCAGGTATAACATTGTTTCAACTGCAAATCAAGCTC |
| | | | | CAACAGGGAACATAGGGAGCTGTTCTCCTCTAGATAA |
| | | | | TAGCTCAGATGCAGCAAAAACAGAATCAATCCCAAAT |
| | | | | TTGGAATCTGATTCAAATGGTACACCTCCCAACCAGG |
| | | | | GTTCCAATGGTAGTAGCAACAATAATGACATGGGATC |
| | | | | CACTACGAATAATGCTTTTACCAAACAAGTGGCTTTTG |
| | | | | CAGAGAGGCCTACAAACAAATCCACAATCAAACTCCA |
| | | | | ATCAAACACTGGTTTCCAACAAGTGCAAAATGGCCAA |
| | | | | GCTTCCCTTCAGACTATTATTCAAGGTAAATATTCGAA |
| | | | | TATGATGCCTAAGTAATTAATTCTAATAAGACACAAGCA |
| | | | | AAGGCCACATACCACTTCATAATAACTTTTCATCGATG |
| | | | | TTGATTTGTTTTACAAAAACTATGGCAGCAGATGCTTC |
| | | | | ACAGTGTGGTTCATCCAATGCATTGAGAGCACCCATG |
| | | | | GAAGGGAATATTAGTAATCACAGTCTCAACAGGAGTG |
| | | | | GGTCAGGTAGTAACCATGGTAGCAACGGACAAAAGA |
| | | | | GAAGCACCAATGCTTCAAACTCCAGAGGGAAAAAGA |
| | | | | CAGAAAGTGAGAGTGTGGTTACTGGAAGAGGGAAAA |
| | | | | CCATTGAAGGAAGTGAATCGGATGGAAATCGATTTGC |
| | | | | ACAAAGAGAAGCTGCTTTGAAAAGATTCCGCCAGAA |
| | | | | GAGGCAAGAAAGATGCTTTGAGAAAAAGGTAAACAG |
| | | | | AAAATCACCCCCCTTATTTTCTCTAAGAAGTAAATCAT |
| | | | | GGAAACAAACAAGTAGGCGTCTGAAAGAAGGAAGTT |
| | | | | TCATTTTCAACACTACACATTTGAACCATCATCTTTGG |
| | | | | GATCCAGTTAGTATGAAGCTTTTTGGGAAAAAAAAAA |
| | | | | GGAAAAGAACAGAAAATTGTTTCCCTGAAATATAAATA |
| | | | | ATTGATCTGTTTCCTTTGAAGTTCCATATTGCTGACCT |
| | | | | GAAGCATTAATTTTTACTTTTTCAGGTGAGATATCAGA |
| | | | | GTAGAAAAAAACTGGCAGAACAAAGACCCCGAATTC |
| | | | | GAGGACAATTTATTAGAAAGGGAATGAATGAAAACAA |
| | | | | GGGAAAAGGCATAAATTACGAACCTGAACCAATTTCA |
| | | | | TAA |
| SEQ ID | AF-causing | 1677 | 1727 | ACATGCAAGGACATCCCTGTAATTAGTGAGTATATTTC |
| NO: 225 | SNP with 25 | | | CTTTATTTTGGAT |
| | bp flanking | | | |
| | genomic DNA | | | |
| | on either side | | | |

PRR37 primers (SEQ ID. 198-205; *a second reverse primer was used to amplify a subset of fragments), coding sequences (SEQ ID. 206-212), predicted protein sequences based on coding sequences (SEQ ID. 213-219), genomic DNA sequences for part of the Response Regulatory domain containing the autoflower-causing SNP (SEQ ID. 220-223), and genomic DNA sequence between start and stop codon based on contigs of the Abacus reference genome (SEQ ID 224). First column: sequence identifier; second column: sequence description; third and fourth columns: sequence start and end positions based on alignment with CBDRx PRR37 sequence starting from start codon (SEQ ID 200, 201, 202, 206, 207, 208, 209, 210, 211, and 212: CDS positions, SEQ ID 213-219: amino acid positions, SEQ ID 203, 204, 205, 220, 221, 222, 223, 224, 225: genomic DNA positions from start codon; **= photosensitive amino acid sequence is partial because only part of the gene was sequenced, not because they are truncated due to a preliminary stop codon); fifth column: sequence. ***= sequences derived from unassembled contigs of the Abacus reference genome: SEQ ID 224 is Abacus PRR37 genomic sequence (start-stop codon) was recovered from unassembled Abacus reference genome (version CsaAba2) contigs; SEQ ID 211_is Abacus predicted mRNA resulting from alignment of the Abacus PRR37 genomic DNA sequence with CBDRx predicted mRNA (CDS between positions 1-139 was sequenced from RNA to confirm the predicted splice site containing the AF-causing SNP); SEQ ID 218 is the translation of SEQ ID 211. SEQ ID 225 is Abacus PRR37 genomic sequence flanking the AF-causing SNP (SNP at position 26 bp in sequence, this SNP is a “G” in the photosensitive accession Abacus).

Alignment of Sanger sequenced fragments of the PRR37 coding sequence (CDS) was performed per accession for each of the two autoflowering (AF1 and AF2) and two photosensitive (PS1 and PS2) accessions. The resulting consensus sequences were subsequently aligned with the CBDRx reference genome DNA sequence (LOC115705128) and predicted mRNA sequence. The CDS alignment revealed two different splice variants for the AF1 autoflowering accession (SEQ ID NOs 206 and 207; Table 8). For the autoflowering accession AF2 three different splice variants were found (SEQ ID NOs 208, 209, and 210; Table 8). The first two splice variants observed in AF1 (X1 and X2; SEQ ID NOs 206 and 207; Table 8) were derived from the same splice sites as the first two splice variants observed in AF2 (X1 and X2; SEQ ID NOs 208 and 209; Table 8). Both photosensitive accessions PS1 and PS2 (SEQ ID NOs 211, and 212), as well as the CBDRx reference genome PRR37 CDS, did not have multiple splice variants. Each of the three splice variants observed in the two autoflowering accessions were due to alternative splicing in the Response Regulatory domain which caused a frameshift leading to a premature stop codon, resulting in truncated protein sequences of 184 amino acids (SEQ ID NOs 213 and 215; Table 8), 151 amino acids (SEQ ID NOs 214 and 216; Table 8), and 176 amino acids (SEQ ID NOs 217; Table 8). All of these protein sequences were truncated inside the Response Regulatory domain (domain in CBDRx between 100-218 amino acids).

Next, the genomic DNA of the Response Regulatory domain region where alternative splicing was observed was sequenced in all four accessions to identify SNP(s) responsible for the alternative splicing in the autoflowering accessions. Genomic DNA sequences revealed a T/T genotype at position 1702 bp from the start codon in the genomic DNA sequence (based on alignment with CBDRx genomic DNA sequence for PRR37), disrupting a canonical splice site in both autoflowering accessions (SEQ ID NOs 220 and 221; Table 8). Both photosensitive accessions had a G/G genotype at position 1702 bp from the start codon in the genomic DNA sequence; the canonical splice site was therefore unchanged in these accessions (SEQ ID NOs 222, 223, and 224; Table 8), as did the CBDRx reference genome. In addition, photosensitive reference genomes Finola and Purple Kush, which like CBDRx had no truncated versions of PRR37, both had a G/G genotype at this position; Van Bakel, Harm, et al. “The draft genome and transcriptome of Cannabis sativa.” Genome biology 12.10 (2011): 1-18). The disappearance of the canonical splice site in the autoflowering accessions led to multiple alternative splice variants during transcription, all ending up as non-functional proteins, truncated at the Response Regulatory domain (FIG. 1). Because a non-functional PRR37 gene no longer can repress flowering pathway genes. In response to photoperiod, plants with this non-functional version of PRR37 are autoflowering.

AN publications and patent applications cited in this specification are herein incorporated by reference as if each individual publication or patent application were specifically and individually indicated to be incorporated by reference.

Although the foregoing invention has been described in some detail byway of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A transgenic Cannabis plant whose genome comprises a homozygous deletion of at least a portion of an endogenous PRR37 gene and wherein the Cannabis plant comprises autoflowering activity.

2. The transgenic Cannabis plant of claim 1 comprising a nucleic acid sequence having at least 90% sequence identity to SEQ ID NO:224, 211, 206, 207, 208, 209, or 210; or
a nucleic acid sequence encoding an amino acid sequence having at least 90% sequence identity to SEQ ID NOs:213, 214, 215, 216, or 217.

3. The transgenic Cannabis plant of claim 1 wherein the homozygous deletion results in a truncated amino acid sequence of a PRR37 protein.

4. (canceled)

5. A cell isolated from the Cannabis plant of claim 1.

6. A cDNA sequence encoding a PRR37 gene from a Cannabis plant comprising a deletion that is capable of conferring autoflowering activity.

7. The cDNA sequence of claim 6 comprising:
(i) a nucleic acid sequence having at least 90% sequence identity to SEQ ID NO:224, 211, 206, 207, 208, 209, or 210; or
(ii) a nucleic acid sequence encoding an amino acid sequence having at least 90% sequence identity to SEQ ID NOs:213, 214, 215, 216, or 217.

8. The cDNA sequence of claim 6 wherein the deletion results in a truncated amino acid sequence of a PRR37 protein.

9. (canceled)

10. An isolated cell whose genome comprises the cDNA sequence of claim 6.

11. A method of making a Cannabis plant conferring autoflowering activity, the method comprising modifying an endogenous PRR37 gene from the Cannabis plant to introduce a homozygous deletion of at least a portion of the endogenous PRR37 gene, thereby conferring the autoflowering activity.

12. The method of claim 11 wherein the modified endogenous PRR37 gene comprises a genomic nucleic acid sequence having at least 90% sequence identity to SEQ ID NO:224 or a protein coding nucleic acid sequence having at least 90% sequence identity to SEQ ID NO:211.

13. The method of claim 11 wherein modifying the endogenous PRR37 gene comprises use of a gene editing technique.

14. The method of claim 13 where the gene editing technique comprises CRISPR technology.

15. A method for breeding autoflowering Cannabis plants, the method comprising:
i) obtaining nucleic acids from a sample Cannabis plant or its germplasm;
(ii) detecting one or more markers that indicate autoflowering activity in the sample;
(iii) selecting the Cannabis plant comprising the one or more markers that indicate autoflowering activity; and
(iv) crossing the Cannabis plant comprising the one or more markers that indicate autoflowering activity, thereby producing one or more progeny plants comprising autoflowering activity.

16-17. (canceled)

18. The method of claim 15 wherein the detecting comprises use of an oligonucleotide probe.

19. The method of claim 15 wherein the marker comprises a polymorphism at position 26 of SEQ ID NO:225.

20. The method of claim 19 wherein the marker comprises a G to T polymorphism at position 26 of SEQ ID NO:225.

21. The method of claim 15 wherein the one or more markers comprises a truncated or deleted protein product of the endogenous PRR37 gene.

22. The method of claim 21 wherein the endogenous PRR37 gene comprises a genomic nucleic acid sequence having at least 90% sequence identity to SEQ ID NO:224 or a protein coding nucleic acid sequence having at least 90% sequence identity to SEQ ID NO:211.

23. (canceled)

24. The method of claim 15 wherein the crossing comprises selfing, sibling crossing, or backcrossing.

25. (canceled)

26. The method of claim 24 wherein the selfing, sibling crossing, or backcrossing comprises marker-assisted selection.

27. (canceled)


SEQUENCE LISTING (225 sequences)

SEQ ID NO: 1 - DNA, 25 bp, Cannabis sativa
  source 1..25: mol_type = genomic DNA
taccctgcgatttgctatggtacta

SEQ ID NO: 2 - DNA, 25 bp, Cannabis sativa
  source 1..25: mol_type = genomic DNA
aaagggtttaatctgtaaatattgt

SEQ ID NO: 3 - DNA, 25 bp, Cannabis sativa
  source 1..25: mol_type = genomic DNA
ctggtgcttctgtgagttgacattg

SEQ ID NO: 4 - DNA, 25 bp, Cannabis sativa
  source 1..25: mol_type = genomic DNA
aaaacgttgctagcatgtatactca

SEQ ID NO: 5 - DNA, 25 bp, Cannabis sativa
  source 1..25: mol_type = genomic DNA
ttttaataagcaagagtattataac

SEQ ID NO: 6 - DNA, 25 bp, Cannabis sativa
  source 1..25: mol_type = genomic DNA
gtccttggccgtctggctcttctaa

SEQ ID NO: 7 - DNA, 25 bp, Cannabis sativa
  source 1..25: mol_type = genomic DNA
aatttataattagttattaaatttt

SEQ ID NO: 8 - DNA, 25 bp, Cannabis sativa
  source 1..25: mol_type = genomic DNA
ggtcatgaattttgctaagatttgc

SEQ ID NO: 9 - DNA, 25 bp, Cannabis sativa
  source 1..25: mol_type = genomic DNA
ttcatcaagtacgaagatacaaatg

SEQ ID NO: 10 - DNA, 25 bp, Cannabis sativa
  source 1..25: mol_type = genomic DNA
tattattatatccggatcatatgta

SEQ ID NO: 11 - DNA, 25 bp, Cannabis sativa
  source 1..25: mol_type = other DNA
ctcctttttattttttggtataggt

SEQ ID NO: 12 - DNA, 25 bp, Cannabis sativa
  source 1..25: mol_type = genomic DNA
acagggactccgtctcagaagtgcg

SEQ ID NO: 13 - DNA, 25 bp, Cannabis sativa
  source 1..25: mol_type = genomic DNA
gtcaatacctggcctctatcatttt

SEQ ID NO: 14 - DNA, 25 bp, Cannabis sativa
  source 1..25: mol_type = genomic DNA
tttgggttttaggaaaagggatgag

SEQ ID NO: 15 - DNA, 25 bp, Cannabis sativa
  source 1..25: mol_type = genomic DNA
atatatgcaattgctggatatgatt

SEQ ID NO: 16 - DNA, 25 bp, Cannabis sativa
  source 1..25: mol_type = genomic DNA
ggaaaaaataaaaattgaagtagga

SEQ ID NO: 17 - DNA, 25 bp, Cannabis sativa
  source 1..25: mol_type = genomic DNA
ggacccaacttgcgctttacctgga

SEQ ID NO: 18 - DNA, 25 bp, Cannabis sativa
  source 1..25: mol_type = genomic DNA
gcacaatacacaccaacctgaatat

SEQ ID NO: 19 - DNA, 25 bp, Cannabis sativa
  source 1..25: mol_type = genomic DNA
ttgtaaactagtgtgtgagaatgtt

SEQ ID NO: 20 - DNA, 25 bp, Cannabis sativa
  source 1..25: mol_type = genomic DNA
taaggctacttaattatattacttc

SEQ ID NO: 21 - DNA, 25 bp, Cannabis sativa
  source 1..25: mol_type = genomic DNA
aacaataaaataaattaggataata

SEQ ID NO: 22 - DNA, 25 bp, Cannabis sativa
  source 1..25: mol_type = genomic DNA
ggcagcaggtggagtaggagaaact

SEQ ID NO: 23 - DNA, 25 bp, Cannabis sativa
  source 1..25: mol_type = genomic DNA
agctctaacagttagagttttaaag

SEQ ID NO: 24 - DNA, 25 bp, Cannabis sativa
  source 1..25: mol_type = genomic DNA
aaagattggtctagcctttgtgttt

SEQ ID NO: 25 - DNA, 25 bp, Cannabis sativa
  source 1..25: mol_type = genomic DNA
gattctgtttgcgacaggcattgac

SEQ ID NO: 26 - DNA, 25 bp, Cannabis sativa
  source 1..25: mol_type = genomic DNA
cggaagaggaggggaggggtcgggg

SEQ ID NO: 27 - DNA, 25 bp, Cannabis sativa
  source 1..25: mol_type = genomic DNA
gggcagcagctagttctagcttata

SEQ ID NO: 28 - DNA, 25 bp, Cannabis sativa
  source 1..25: mol_type = genomic DNA
tgattttgcattctcaactttcttc

SEQ ID NO: 29 - DNA, 25 bp, Cannabis sativa
  source 1..25: mol_type = genomic DNA
caaccctttgtatacttggctccac

SEQ ID NO: 30 - DNA, 25 bp, Cannabis sativa
  source 1..25: mol_type = genomic DNA
gaattatttgagcgaatattatata

SEQ ID NO: 31 - DNA, 25 bp, Cannabis sativa
  source 1..25: mol_type = genomic DNA
cattcattctagcctcaaaacttta

SEQ ID NO: 32 - DNA, 25 bp, Cannabis sativa
  source 1..25: mol_type = genomic DNA
tcaaatcaaattgataaatttcatg

SEQ ID NO: 33 - DNA, 25 bp, Cannabis sativa
  source 1..25: mol_type = genomic DNA
tatgggctttgagaaaattggcaca

SEQ ID NO: 34 - DNA, 25 bp, Cannabis sativa
  source 1..25: mol_type = genomic DNA
ggaacttgctcggcttagtgacata

SEQ ID NO: 35 - DNA, 25 bp, Cannabis sativa
  source 1..25: mol_type = genomic DNA
ttcatcagtgacctgaattggtgat

SEQ ID NO: 36 - DNA, 25 bp, Cannabis sativa
  source 1..25: mol_type = genomic DNA
atatgttgaagatgtgtccgattcc

SEQ ID NO: 37 - DNA, 25 bp, Cannabis sativa
  source 1..25: mol_type = genomic DNA
ccaactgaaaaagcttgcttggtgg

SEQ ID NO: 38 - DNA, 25 bp, Cannabis sativa
  source 1..25: mol_type = genomic DNA
ccggtaactttgtcgtcgtcagcat

SEQ ID NO: 39 - DNA, 25 bp, Cannabis sativa
  source 1..25: mol_type = genomic DNA
gtacaaatgggcactcatcagtcag

SEQ ID NO: 40 - DNA, 25 bp, Cannabis sativa
  source 1..25: mol_type = genomic DNA
aaatacacaaactaatagctcgact

SEQ ID NO: 41 - DNA, 25 bp, Cannabis sativa
  source 1..25: mol_type = genomic DNA
cctaatgtttctaatctttgtttca

SEQ ID NO: 42 - DNA, 25 bp, Cannabis sativa
  source 1..25: mol_type = genomic DNA
tgagtatgtaaatcatgtttctaac

SEQ ID NO: 43 - DNA, 25 bp, Cannabis sativa
  source 1..25: mol_type = genomic DNA
tctgaaactcaagcctctctgggcc

SEQ ID NO: 44 - DNA, 25 bp, Cannabis sativa
  source 1..25: mol_type = genomic DNA
cttagggaccaccaatgtatcaaag

SEQ ID NO: 45 - DNA, 25 bp, Cannabis sativa
  source 1..25: mol_type = genomic DNA
gcagcagcacccccttgcttgaaaa

SEQ ID NO: 46 - DNA, 25 bp, Cannabis sativa
  source 1..25: mol_type = genomic DNA
cggcggaagtggaggacggttcgga

SEQ ID NO: 47 - DNA, 25 bp, Cannabis sativa
  source 1..25: mol_type = genomic DNA
ttgttcttgggtattagaagcaagg

SEQ ID NO: 48 - DNA, 25 bp, Cannabis sativa
  source 1..25: mol_type = genomic DNA
agatgattttgttaaacattgtagt

SEQ ID NO: 49 - DNA, 25 bp, Cannabis sativa
  source 1..25: mol_type = genomic DNA
ttcatcttcaaccctatcattatcg

SEQ ID NO: 50 - DNA, 25 bp, Cannabis sativa
  source 1..25: mol_type = genomic DNA
ttagacttgtgctcctttggatgca

SEQ ID NO: 51 - DNA, 25 bp, Cannabis sativa
  source 1..25: mol_type = genomic DNA
gaacctagaccaggccaaccacagg

SEQ ID NO: 52 - DNA, 25 bp, Cannabis sativa
  source 1..25: mol_type = genomic DNA
cgaaaggggaaacaactacgatatt

SEQ ID NO: 53 - DNA, 25 bp, Cannabis sativa
  source 1..25: mol_type = genomic DNA
tcaacctataaatataattgtgtat

SEQ ID NO: 54 - DNA, 25 bp, Cannabis sativa
  source 1..25: mol_type = genomic DNA
tagccaaacctaccaatttgaatgc

SEQ ID NO: 55 - DNA, 25 bp, Cannabis sativa
  source 1..25: mol_type = genomic DNA
acaaaggtgtttgtcaatgtaatga

SEQ ID NO: 56 - DNA, 25 bp, Cannabis sativa
  source 1..25: mol_type = genomic DNA
ttaactatggcctgcaggtcaattc

SEQ ID NO: 57 - DNA, 25 bp, Cannabis sativa
  source 1..25: mol_type = genomic DNA
acactttacttagtatataatagat

SEQ ID NO: 58 - DNA, 25 bp, Cannabis sativa
  source 1..25: mol_type = genomic DNA
tgtctcagagacgacaagaatgtct

SEQ ID NO: 59 - DNA, 25 bp, Cannabis sativa
  source 1..25: mol_type = genomic DNA
atgtgcccgaaaagctataatttca

SEQ ID NO: 60 - DNA, 25 bp, Cannabis sativa
  source 1..25: mol_type = genomic DNA
gaggagaatcagttggttttcaagg

SEQ ID NO: 61 - DNA, 25 bp, Cannabis sativa
  source 1..25: mol_type = genomic DNA
gcaaccatagacattggataacttg

SEQ ID NO: 62 - DNA, 25 bp, Cannabis sativa
  source 1..25: mol_type = genomic DNA
tatacaatgccaggcacatcccagc

SEQ ID NO: 63 - DNA, 25 bp, Cannabis sativa
  source 1..25: mol_type = genomic DNA
attaagataatagatcactgatggc

SEQ ID NO: 64 - DNA, 25 bp, Cannabis sativa
  source 1..25: mol_type = genomic DNA
gttggaagcctcgggggcaccggaa

SEQ ID NO: 65 - DNA, 25 bp, Cannabis sativa
  source 1..25: mol_type = genomic DNA
aagatattaacactgcggattggat

SEQ ID NO: 66 - DNA, 25 bp, Cannabis sativa
  source 1..25: mol_type = genomic DNA
ccaaatcaccatgtgcaacacccca

SEQ ID NO: 67 - DNA, 25 bp, Cannabis sativa
  source 1..25: mol_type = genomic DNA
acatagggtctgagattgtcgttcg

SEQ ID NO: 68 - DNA, 25 bp, Cannabis sativa
  source 1..25: mol_type = genomic DNA
aggcttatccttggacgcctttctt

SEQ ID NO: 69 - DNA, 25 bp, Cannabis sativa
  source 1..25: mol_type = genomic DNA
atgacattgtccttaagcttgggac

SEQ ID NO: 70 - DNA, 25 bp, Cannabis sativa
  source 1..25: mol_type = genomic DNA
ttgccgtatttgtaattagttttag

SEQ ID NO: 71 - DNA, 25 bp, Cannabis sativa
  source 1..25: mol_type = genomic DNA
tgaactgggctcgcacattcttttt

SEQ ID NO: 72 - DNA, 25 bp, Cannabis sativa
  source 1..25: mol_type = genomic DNA
ctcttttttcttgcatgaatccctc

SEQ ID NO: 73 - DNA, 25 bp, Cannabis sativa
  source 1..25: mol_type = genomic DNA
cttttataaattcctgtgtctcttg

SEQ ID NO: 74 - DNA, 25 bp, Cannabis sativa
  source 1..25: mol_type = genomic DNA
gatatttacaatgatttatatagtt

SEQ ID NO: 75 - DNA, 25 bp, Cannabis sativa
  source 1..25: mol_type = genomic DNA
ttcatacaataggttggattgcaat

SEQ ID NO: 76 - DNA, 25 bp, Cannabis sativa
  source 1..25: mol_type = genomic DNA
gaaagaatgttataaaatttacctg

SEQ ID NO: 77 - DNA, 25 bp, Cannabis sativa
  source 1..25: mol_type = genomic DNA
atggcctgagttttccaacctcgtt

SEQ ID NO: 78 - DNA, 25 bp, Cannabis sativa
  source 1..25: mol_type = genomic DNA
cggcggagatgaatgagtattagaa

SEQ ID NO: 79 - DNA, 25 bp, Cannabis sativa
  source 1..25: mol_type = genomic DNA
ggttctgatcgtcgtgatgggaagt

SEQ ID NO: 80 - DNA, 25 bp, Cannabis sativa
  source 1..25: mol_type = genomic DNA
gaaggatgcccctaggaggcaccga

SEQ ID NO: 81 - DNA, 25 bp, Cannabis sativa
  source 1..25: mol_type = genomic DNA
gaaccgtgatttcctcattggttgc

SEQ ID NO: 82 - DNA, 25 bp, Cannabis sativa
  source 1..25: mol_type = genomic DNA
cctttcaacatactacttccacctt

SEQ ID NO: 83 - DNA, 25 bp, Cannabis sativa
  source 1..25: mol_type = genomic DNA
tggttcagcgagttcctgaaccatt

SEQ ID NO: 84 - DNA, 25 bp, Cannabis sativa
  source 1..25: mol_type = genomic DNA
aggattccctctctgcgtctaactc

SEQ ID NO: 85 - DNA, 25 bp, Cannabis sativa
  source 1..25: mol_type = genomic DNA
tattataaatgacccaataatatct

SEQ ID NO: 86 - DNA, 25 bp, Cannabis sativa
  source 1..25: mol_type = genomic DNA
aacagaggtattgaaagggaagccc

SEQ ID NO: 87 - DNA, 25 bp, Cannabis sativa
  source 1..25: mol_type = genomic DNA
tgcaaggaagtttgctctttgcatc

SEQ ID NO: 88 - DNA, 25 bp, Cannabis sativa
  source 1..25: mol_type = genomic DNA
agacaatggtgtcgagaacccatcg

SEQ ID NO: 89 - DNA, 25 bp, Cannabis sativa
  source 1..25: mol_type = genomic DNA
aagttatttaattacaataagtatt

SEQ ID NO: 90 - DNA, 25 bp, Cannabis sativa
  source 1..25: mol_type = genomic DNA
taggggccttatatgacagcgctta

SEQ ID NO: 91 - DNA, 25 bp, Cannabis sativa
  source 1..25: mol_type = genomic DNA
ttccatctaacctaagagtacaaac

SEQ ID NO: 92 - DNA, 25 bp, Cannabis sativa
  source 1..25: mol_type = genomic DNA
taaaatttattagcctccgaagaaa

SEQ ID NO: 93 - DNA, 25 bp, Cannabis sativa
  source 1..25: mol_type = genomic DNA
gttagtcctcactccaggagctttt

SEQ ID NO: 94 - DNA, 25 bp, Cannabis sativa
  source 1..25: mol_type = genomic DNA
atgaatcaaactaagcataatttaa

SEQ ID NO: 95 - DNA, 25 bp, Cannabis sativa
  source 1..25: mol_type = genomic DNA
ttcttgtttgaaattggggttaaac

SEQ ID NO: 96 - DNA, 25 bp, Cannabis sativa
  source 1..25: mol_type = genomic DNA
tgttttatgtttggtcttaccttag

SEQ ID NO: 97 - DNA, 25 bp, Cannabis sativa
  source 1..25: mol_type = genomic DNA
tgcctgatatgtgcatagcacacac

SEQ ID NO: 98 - DNA, 25 bp, Cannabis sativa
  source 1..25: mol_type = genomic DNA
gatggtaattggttgtcctcctcat

SEQ ID NO: 99 - DNA, 25 bp, Cannabis sativa
  source 1..25: mol_type = genomic DNA
tattggtttacttgctgaagcccaa

SEQ ID NO: 100 - DNA, 25 bp, Cannabis sativa
  source 1..25: mol_type = genomic DNA
actagccacactagaaagccttgat

SEQ ID NO: 101 - DNA, 25 bp, Cannabis sativa
  source 1..25: mol_type = genomic DNA
aaaggtttagctcgagtgtcatctg

SEQ ID NO: 102 - DNA, 25 bp, Cannabis sativa
  source 1..25: mol_type = genomic DNA
ttgatgagggagcaaaatacttttc

SEQ ID NO: 103 - DNA, 25 bp, Cannabis sativa
  source 1..25: mol_type = genomic DNA
aggtggatatctcactacagataag

SEQ ID NO: 104 - DNA, 25 bp, Cannabis sativa
  source 1..25: mol_type = genomic DNA
aacatgattcctaatagattcacct

SEQ ID NO: 105 - DNA, 25 bp, Cannabis sativa
  source 1..25: mol_type = genomic DNA
acagaggatataatacaggttttgg

SEQ ID NO: 106 - DNA, 25 bp, Cannabis sativa
  source 1..25: mol_type = genomic DNA
tacctctcgatcgccttcaatgcat

SEQ ID NO: 107 - DNA, 25 bp, Cannabis sativa
  source 1..25: mol_type = genomic DNA
tcctcctaatgcgacccacttgatt

SEQ ID NO: 108 - DNA, 25 bp, Cannabis sativa
  source 1..25: mol_type = genomic DNA
ggaagttactcccggaggccattga

SEQ ID NO: 109 - DNA, 25 bp, Cannabis sativa
  source 1..25: mol_type = genomic DNA
atctaagatcctggtaaaatatata

SEQ ID NO: 110 - DNA, 25 bp, Cannabis sativa
  source 1..25: mol_type = genomic DNA
gtgaagttgtttaatgagttttaaa

SEQ ID NO: 111 - DNA, 25 bp, Cannabis sativa
  source 1..25: mol_type = genomic DNA
atgttccacaatccctaaaacattt

SEQ ID NO: 112 - DNA, 25 bp, Cannabis sativa
  source 1..25: mol_type = genomic DNA
ggtcatttgctggcctcatctgatg

SEQ ID NO: 113 - DNA, 25 bp, Cannabis sativa
  source 1..25: mol_type = genomic DNA
gcttgtaacaaagcatttaatattt

SEQ ID NO: 114 - DNA, 25 bp, Cannabis sativa
  source 1..25: mol_type = genomic DNA
gtgctcatgcctcaaatgaagctaa

SEQ ID NO: 115 - DNA, 25 bp, Cannabis sativa
  source 1..25: mol_type = genomic DNA
gccaagtcctcagcatggtaatcta

SEQ ID NO: 116 - DNA, 25 bp, Cannabis sativa
  source 1..25: mol_type = genomic DNA
atccattttccaggtataggctggc

SEQ ID NO: 117 - DNA, 25 bp, Cannabis sativa
  source 1..25: mol_type = genomic DNA
atgacgtaatttgtctccagtaatg

SEQ ID NO: 118 - DNA, 25 bp, Cannabis sativa
  source 1..25: mol_type = genomic DNA
atatgttgaagatgtgtccgattcc

SEQ ID NO: 119 - DNA, 25 bp, Cannabis sativa
  source 1..25: mol_type = genomic DNA
tcatggaatctaaaagggaatcgag

SEQ ID NO: 120 - DNA, 25 bp, Cannabis sativa
  source 1..25: mol_type = genomic DNA
ttattccaacttaaacagattaagt

SEQ ID NO: 121 - DNA, 25 bp, Cannabis sativa
  source 1..25: mol_type = genomic DNA
gtttcaatggtctaagttcgtatca

SEQ ID NO: 122 - DNA, 25 bp, Cannabis sativa
  source 1..25: mol_type = genomic DNA
agtgggatttatggcaggcctagca

SEQ ID NO: 123 - DNA, 25 bp, Cannabis sativa
  source 1..25: mol_type = genomic DNA
ggcaactcaaaggcagagattgtcc

SEQ ID NO: 124 - DNA, 25 bp, Cannabis sativa
  source 1..25: mol_type = genomic DNA
ttaacttgtctccacatgtgacatg

SEQ ID NO: 125 - DNA, 25 bp, Cannabis sativa
  source 1..25: mol_type = genomic DNA
tagattgggtcacatttttgaaaca

SEQ ID NO: 126 - DNA, 25 bp, Cannabis sativa
  source 1..25: mol_type = genomic DNA
tgtatatagcacgaaatgttacttt

SEQ ID NO: 127 - DNA, 25 bp, Cannabis sativa
  source 1..25: mol_type = genomic DNA
cctacatctacatattgggatgcat

SEQ ID NO: 128 - DNA, 25 bp, Cannabis sativa
  source 1..25: mol_type = genomic DNA
tccctaacatctttaatgtgcttga

SEQ ID NO: 129 - DNA, 25 bp, Cannabis sativa
  source 1..25: mol_type = genomic DNA
cgtacaaaattcctcactgtacgcc

SEQ ID NO: 130 - DNA, 25 bp, Cannabis sativa
  source 1..25: mol_type = genomic DNA
ttaggatctattctaatttagatcc

SEQ ID NO: 131 - DNA, 25 bp, Cannabis sativa
  source 1..25: mol_type = genomic DNA
tcccatgatcgtgacgctccattca

SEQ ID NO: 132 - DNA, 25 bp, Cannabis sativa
  source 1..25: mol_type = genomic DNA
ttccctttctcaatatgtatttaac

SEQ ID NO: 133 - DNA, 25 bp, Cannabis sativa
  source 1..25: mol_type = genomic DNA
aacaggagaagataaattaagaata

SEQ ID NO: 134 - DNA, 25 bp, Cannabis sativa
  source 1..25: mol_type = genomic DNA
aaccccagaaactgctctctaaaat

SEQ ID NO: 135 - DNA, 25 bp, Cannabis sativa
  source 1..25: mol_type = genomic DNA
cgggggtgatgtctgcgactgtctt

SEQ ID NO: 136 - DNA, 25 bp, Cannabis sativa
  source 1..25: mol_type = genomic DNA
atcatcactcttgtcttttttcttt

SEQ ID NO: 137 - DNA, 25 bp, Cannabis sativa
  source 1..25: mol_type = genomic DNA
tttatttattatcctagtcttcaag

SEQ ID NO: 138 - DNA, 25 bp, Cannabis sativa
  source 1..25: mol_type = genomic DNA
gtgaagctcactcaaactagatggt

SEQ ID NO: 139 - DNA, 25 bp, Cannabis sativa
  source 1..25: mol_type = genomic DNA
atgtacattaattatgaatagaacc

SEQ ID NO: 140 - DNA, 25 bp, Cannabis sativa
  source 1..25: mol_type = genomic DNA
gaaaatcaccgtgaggagtggggtt

SEQ ID NO: 141 - DNA, 25 bp, Cannabis sativa
  source 1..25: mol_type = genomic DNA
gccaggagagaggttactgatacta

SEQ ID NO: 142 - DNA, 25 bp, Cannabis sativa
  source 1..25: mol_type = genomic DNA
aatctcttctttagtttgtttcatt

SEQ ID NO: 143 - DNA, 25 bp, Cannabis sativa
  source 1..25: mol_type = genomic DNA
gattgttattattattttataaact

SEQ ID NO: 144 - DNA, 25 bp, Cannabis sativa
  source 1..25: mol_type = genomic DNA
tttaaggagagagatcgaccatttt

SEQ ID NO: 145 - DNA, 25 bp, Cannabis sativa
  source 1..25: mol_type = genomic DNA
cctcaccatcagaaggtacctcacc

SEQ ID NO: 146 - DNA, 25 bp, Cannabis sativa
  source 1..25: mol_type = genomic DNA
tgaccttgagaagaaatctcccacc

SEQ ID NO: 147 - DNA, 25 bp, Cannabis sativa
  source 1..25: mol_type = genomic DNA
agctgggttttcctcaagcgaagtt

SEQ ID NO: 148 - DNA, 25 bp, Cannabis sativa
  source 1..25: mol_type = genomic DNA
ataacctagcttgttgaggtctttt

SEQ ID NO: 149 - DNA, 25 bp, Cannabis sativa
  source 1..25: mol_type = genomic DNA
gccccagcaaccgttgtattctcct

SEQ ID NO: 150 - DNA, 25 bp, Cannabis sativa
  source 1..25: mol_type = genomic DNA
tatttttgaagggttatcaaatctc

SEQ ID NO: 151 - DNA, 25 bp, Cannabis sativa
  source 1..25: mol_type = genomic DNA
tgagattcttcaaagaataacacca

SEQ ID NO: 152 - DNA, 25 bp, Cannabis sativa
  source 1..25: mol_type = genomic DNA
caaacatttttcgataagtatacct

SEQ ID NO: 153 - DNA, 25 bp, Cannabis sativa
  source 1..25: mol_type = genomic DNA
tttaattaaattcaattaattaagt

SEQ ID NO: 154 - DNA, 25 bp, Cannabis sativa
  source 1..25: mol_type = genomic DNA
tcccacatatacctgcccagttctt

SEQ ID NO: 155 - DNA, 25 bp, Cannabis sativa
  source 1..25: mol_type = genomic DNA
ataatcaaaagtgtcatctaagaca

SEQ ID NO: 156 - DNA, 25 bp, Cannabis sativa
  source 1..25: mol_type = genomic DNA
ataacccaattttatggtgattcct

SEQ ID NO: 157 - DNA, 25 bp, Cannabis sativa
  source 1..25: mol_type = genomic DNA
agcccattggtacgaataatttgaa

SEQ ID NO: 158 - DNA, 25 bp, Cannabis sativa
  source 1..25: mol_type = genomic DNA
gctgttgtaagatattggcaaggta

SEQ ID NO: 159 - DNA, 25 bp, Cannabis sativa
  source 1..25: mol_type = genomic DNA
tgtgagcatccacaaacaaattaat

SEQ ID NO: 160 - DNA, 25 bp, Cannabis sativa
  source 1..25: mol_type = genomic DNA
tgaaacatttctatatttggggttg

SEQ ID NO: 161 - DNA, 25 bp, Cannabis sativa
  source 1..25: mol_type = genomic DNA
tgcaactttgtagaaaaggtctttt

SEQ ID NO: 162 - DNA, 25 bp, Cannabis sativa
  source 1..25: mol_type = genomic DNA
ttctaaccactgtacaaggttatat

SEQ ID NO: 163 - DNA, 25 bp, Cannabis sativa
  source 1..25: mol_type = genomic DNA
gttactaaatgtgcaacatatttat

SEQ ID NO: 164 - DNA, 25 bp, Cannabis sativa
  source 1..25: mol_type = genomic DNA
aatgtccaagcacgcaacatctcca

SEQ ID NO: 165 - DNA, 25 bp, Cannabis sativa
  source 1..25: mol_type = genomic DNA
aagcttgatataaagggaagcctct

SEQ ID NO: 166 - DNA, 25 bp, Cannabis sativa
  source 1..25: mol_type = genomic DNA
atccataggcacagcatcctcattc

SEQ ID NO: 167 - DNA, 25 bp, Cannabis sativa
  source 1..25: mol_type = genomic DNA
gcaggaaatgaagtcggaatatcca

SEQ ID NO: 168 - DNA, 25 bp, Cannabis sativa
  source 1..25: mol_type = genomic DNA
tacactttgaaaagaagaattaaaa

SEQ ID NO: 169 - DNA, 25 bp, Cannabis sativa
  source 1..25: mol_type = genomic DNA
tacgacaagccgcgagcacgaatat

SEQ ID NO: 170 - DNA, 25 bp, Cannabis sativa
  source 1..25: mol_type = genomic DNA
tgtgacactttaatttttacaaaaa

SEQ ID NO: 171 - DNA, 25 bp, Cannabis sativa
  source 1..25: mol_type = genomic DNA
ggacgagtcaacaacagagatggga

SEQ ID NO: 172 - DNA, 20 bp, Cannabis sativa
  source 1..20: mol_type = genomic DNA
gccgattcaacctacgggaa

SEQ ID NO: 173 - DNA, 20 bp, Cannabis sativa
  source 1..20: mol_type = genomic DNA
cttgcagcctctagttcgct

SEQ ID NO: 174 - DNA, 20 bp, Cannabis sativa
  source 1..20: mol_type = genomic DNA
tcagcaaatggacagagtgc

SEQ ID NO: 175 - DNA, 20 bp, Cannabis sativa
  source 1..20: mol_type = genomic DNA
attccaccaccggaataatg

SEQ ID NO: 176 - DNA, 20 bp, Cannabis sativa
  source 1..20: mol_type = genomic DNA
gaaggctggttttgctggtg

SEQ ID NO: 177 - DNA, 20 bp, Cannabis sativa
  source 1..20: mol_type = genomic DNA
tcagcaatgccagggaacat

SEQ ID NO: 178 - DNA, 27 bp, Cannabis sativa
  source 1..27: mol_type = genomic DNA
ccaaactagatagattattcttctgcc

SEQ ID NO: 179 - DNA, 21 bp, Cannabis sativa
  source 1..21: mol_type = genomic DNA
aacaaccgaagaaccagagga

SEQ ID NO: 180 - DNA, 20 bp, Cannabis sativa
  source 1..20: mol_type = genomic DNA
cgattcaacctacgggaaga

SEQ ID NO: 181 - DNA, 20 bp, Cannabis sativa
  source 1..20: mol_type = genomic DNA
cagcatgagctgtgtcgaat

SEQ ID NO: 182 - DNA, 20 bp, Cannabis sativa
  source 1..20: mol_type = genomic DNA
aggtggattcgacacagctc

SEQ ID NO: 183 - DNA, 20 bp, Cannabis sativa
  source 1..20: mol_type = genomic DNA
ccaagccaaaggcataatgt

SEQ ID NO: 184 - DNA, 20 bp, Cannabis sativa
  source 1..20: mol_type = genomic DNA
attcgacagcgaactagagg

SEQ ID NO: 185 - DNA, 20 bp, Cannabis sativa
  source 1..20: mol_type = genomic DNA
acaagggaggcattcagaga

SEQ ID NO: 186 - DNA, 23 bp, Cannabis sativa
  source 1..23: mol_type = genomic DNA
tgcatggactataatggtgagtg

SEQ ID NO: 187 - DNA, 20 bp, Cannabis sativa
  source 1..20: mol_type = genomic DNA
ggcatcacaaatggaagtca

SEQ ID NO: 188 - DNA, 20 bp, Cannabis sativa
  source 1..20: mol_type = genomic DNA
acacttcggcagagcaattt

SEQ ID NO: 189 - DNA, 20 bp, Cannabis sativa
  source 1..20: mol_type = genomic DNA
ggtctgattcaggtgccaat

SEQ ID NO: 190 - DNA, 20 bp, Cannabis sativa
  source 1..20: mol_type = genomic DNA
gggaagcagaatccaagatg

SEQ ID NO: 191 - DNA, 20 bp, Cannabis sativa
  source 1..20: mol_type = genomic DNA
aagccagcagtgagagaagc

SEQ ID NO: 192 - DNA, 20 bp, Cannabis sativa
  source 1..20: mol_type = genomic DNA
gctgaggcagcttcgtaaat

SEQ ID NO: 193 - DNA, 20 bp, Cannabis sativa
  source 1..20: mol_type = genomic DNA
cgcttccagaatcactgtca

SEQ ID NO: 194 - DNA, 20 bp, Cannabis sativa
  source 1..20: mol_type = genomic DNA
tcagcaaatggacagagtgc

SEQ ID NO: 195 - DNA, 20 bp, Cannabis sativa
  source 1..20: mol_type = genomic DNA
attccaccaccggaataatg

SEQ ID NO: 196 - protein, 1451 aa, Cannabis sativa
MTSMDHIFICMDYNGEWKITDNCIWEWFGTGCNKEFVVDHSIKFHQLVNKVYEKIGVDQNLYKIEITHKVAGETFNKMVPSKICGDSDVEDLLKELYKVKEVIPLYVCIKKNNDKGKTKFVNDDDGDGNELTGDDVEFDCDDDVFDNRSFYDNYFGCHIIDQVPNDPSYILNEDIPQSGEGIIGSNPTPDDIVHESGNNELQECDKVVEENNDVIENDIIVEGGQIIPYQHYDMFMGNVAQCSARGGAHQLRFSGCMMEVSVCEVKGKMDHHEDEGRVGGGENIGKQNEEEAVARLEEMKKSIEGKITLRQSNLNPERPDSGFLRTLDSSIRRNTAVIKKLKQINEEQREGLLDDLRSVNLSKFVSEAVTSICDAKLRSSDIQSAVQICSLLHQRYKDFSPSLVQGLLKVFFPGKSGDDSDTDRNQKAMKKRSTLKLLLELYFVGVIEDSAIFVSIIKDLTSIEHLKDRDTTQTNLSLLASFARQGRIFLGLPLSGQEVYEEFLKGLNITSDQKKIFRKALHAYYEAASELLQTEHTSLRQLEHENAKILNAKGELSDENVASYEKLRKSYDQLYRNISSLAEALDMQPPVMPEDGHTTRVTSGDDASSNSTGKDSSALEAIWDDEDTRSFYECLPDLRAFVPAVLLGEAESKMNEQSVKTQEQSTELAPESDQVQQTAPDSAEISTDSGASQEGRSTEKGKEKEEKEKDKSKDPEKEKGKEKDADKKGDTEKEKLKSIEGTNLDALLQRLPGCVSRDLIDQLTVEFCYLNSKASRKKLVRALFNVPRTSLELLPYYSRMVATLSTCMKDVSSMLLQMLEEEFNFLINKKDQMNIETKIRNIRFIGELCKFKIAPAGLVFSCLKACLDDFSHHNIDVACNLLETCGRFLYRSPETTVRMANMLEILMRLKNVKNLDPRHSTLVENAYYLCKPPERSARIAKVRPPLHQYIRKLLFSDLDKSTIEHVLRQLRKLPWSECEPYLLKCFMKVHRGKYGQIHLIASLTAGLSRYHDEFAVAVVDEVLEEIRVGLELNDYGMQQRRLAHMRFLGELYNYEHVDSSVIFETLYLILVFGHGSPEQDLLDPPEDCFRMRMVITLLETCGHYFDRGSSKRKLDRFLIHFQRYILSKGALPLDIEFDLQDLFADLRPNMTRYSSIEEVTLALVELEEHERTLPSDKTSSEKHSDSEIRSSFNSISANGQSAVNGNEGNGRLHDGLGDSDSDSGSGTLDQEGRDEEELDDENHDEECDTDEEDDDGGGPASDEDEVHVRQKVMEVDPLEAATFEQELKAVMQESMDQRRQELRGRPTLNMMIPMNVFEGSTRGVGGESGDEALDEEGGGIKDVQVKVLVKRGSKQQTRQMYIPRDCSLVQSTKQKEAAELEEKQDIKRLVLEYNDREEEELNGLGTQTLNHMQGSGSRGSTRGHLWEGSSGRGGTRHRHYSGGGIFYNRKK

SEQ ID NO: 197 - protein, 551 aa, Cannabis sativa
MMLDLNVNINNGADSTYGKTKERGAELVIMEVEDKLQKGSTTQIMEDSGSSGSSVVNVEIDALSSTTTITTSNGVFREEDSSINVTNTTSSTFFFDIMKREKDCNNGATAGKETNNISPPGFLTRSFFPVAGEKVGNQFVEAGSGSSSSRPQWLNLSFADSGGGGAAVQPPADVKVLQQKQQIKKSRRGPRSRSSQYRGVTFYRRTGRWESHIWDCGKQVYLGGFDTAHAAARAYDRAAIKFRGVDADINFNVTDYDEDMMQMKNLSKEEFVQILRRQSTGFSRGSSKYRGVTLHKCGRWEARMGQFLGKKYIYLGLFDSELEAARAYDKAAIKCNGRDAITNFEHSTYQGEIILDTNAQGNDHNLDLNLGISPPCDGPKGREYSFGLGDTRVHWNPREGPHRIRPIMIDGQSSPHILPPNHAASATCSGVYPAFLPKHEEMRAMADDHQKRIEAANSSQGFLNWAWKIHGNGSNTTTTTTSSCVTAMPTFSIAASSGFSSSTSLAALSATNNINPQANFVQNNICLSPSMPITTTNSVTNNFHNSQIHRG

SEQ ID NO: 198 - DNA, 22 bp, Cannabis sativa
  source 1..22: mol_type = genomic DNA
gggactttgatcagaacacaca

SEQ ID NO: 199 - DNA, 21 bp, Cannabis sativa
  source 1..21: mol_type = genomic DNA
gctgaaacgtactgaccttcg

SEQ ID NO: 200 - DNA, 20 bp, Cannabis sativa
  source 1..20: mol_type = genomic DNA
agctcccgtaaccaatgatg

SEQ ID NO: 201 - DNA, 20 bp, Cannabis sativa
  source 1..20: mol_type = genomic DNA
catgttggtcagcccttttt

SEQ ID NO: 202 - DNA, 20 bp, Cannabis sativa
  source 1..20: mol_type = genomic DNA
tggagacacccatcagatca

SEQ ID NO: 203 - DNA, 20 bp, Cannabis sativa
  source 1..20: mol_type = genomic DNA
ggcagacaagcttggaaagt

SEQ ID NO: 204 - DNA, 20 bp, Cannabis sativa
  source 1..20: mol_type = genomic DNA
ctcatcattgctgccactgt

SEQ ID NO: 205 - DNA, 20 bp, Cannabis sativa
  source 1..20: mol_type = genomic DNA
tgtggcattttctccaaaca

SEQ ID NO: 206 - RNA, 1090 nt, Cannabis sativa
  source 1..1090: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattattgatgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagagctgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatg

SEQ ID NO: 207 - RNA, 1004 nt, Cannabis sativa
  source 1..1004: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatcttggatcaatgaagatgtgcaacaacatgtcagcaatggggagattggaacagtacaggctctggagaggagtcattctggccagcggaggtctcagcaacagcctcaaggacatttggttcgctgggagaggttcctrgctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgagtgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatg

SEQ ID NO: 208 - RNA, 1089 nt, Cannabis sativa
  source 1..1089: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattattgatgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaaccatccaaggcaagaagcctttggaaacaactgggtacctgaaaatgcaacagtaacaccacgcccacataatgatgagcttgacaaaaaagtcatgggaaaagacttgaaaataggattacctagccttcttgaggacacaagtgaaaaagggctgaccaacat

SEQ ID NO: 209 - RNA, 851 nt, Cannabis sativa
  source 1..851: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgagtgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaa

SEQ ID NO: 210 - RNA, 1079 nt, Cannabis sativa
  source 1..1079: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatcccttgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaggacacaagtgaaaaagggctgaccaacatg

SEQ ID NO: 211 - RNA, 2172 nt, Cannabis sativa
  source 1..2172: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattatgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttgaaaagattccgccagaagaggcaagaaagatgctttgagaaaaaggtgagatatcagagtagaaaaaaactggcagaacaaagaccccgaattcgaggacaatttattagaaagggaatgaatgaaaacaagggaaaaggcataaattacgaacctgaaccaatttcataa

SEQ ID NO: 212 - RNA, 1182 nt, Cannabis sativa
  source 1..1182: mol_type = mRNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgargatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattatgatgtcttctcatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggwatatggattgaaaagcctttaaagtcaagaactgtggaacattcwgacaacaacagtggcagcaatgatgaggatgayactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccg

SEQ ID NO: 213 - protein, 184 aa, Cannabis sativa
MKMVQMNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIPVIIDDVFT

SEQ ID NO: 214 - protein, 151 aa, Cannabis sativa
MKMVQMNNKAPVTNDELTELNHRIQDGKKERRERVTRERQGLSEEHESWINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTE

SEQ ID NO: 215 - protein, 184 aa, Cannabis sativa; identical to SEQ ID NO: 213

SEQ ID NO: 216 - protein, 151 aa, Cannabis sativa
MKMVQMNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTE

SEQ ID NO: 217 - protein, 176 aa, Cannabis sativa
MKMVQMNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIP

SEQ ID NO: 218 - protein, 723 aa, Cannabis sativa
MKMVQMNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIPVIMMSSHDSRSMVFKCLSKGAVDFLVKPIRKNELKNLWQHVWRKCHISSNSGSESGIWIEKPLKSRTVEHSDNNSGSNDEDDTDSIGLNFRNESDSGTQSSWTKRAEVDSPQAVSWEQFADLPDSTNHQVNHPRQEAFGNNWVPENATVTPRPHNDELDKKVMGKDLKIGLPSLLEDTSEKGLTNMEGTNKDKCSELNSKKDDQEQEKRELDLNNEEPSAEKTQAVDLMGVSNYSIDPHMESGVLDVPNELSKAACMRDNANHENKETPFFELILKRPRDIQDTGTSAHDRNVLRHSDISAFSRYNIVSTANQAPTGNIGSCSPLDNSSDAAKTESIPNLQSDSNGTPPNQGSNGSSNNNDMGSTTNNAFTKQVAFAERPTNKSTIKLQSNTGFQQVQNGQASLQTIIQDASQCGSSNALRAPMEGNISNHSLNRSGSGSNHGSNGQKRSTNASNSRGKKTESESVVTGRGKTIEGSESDGNRFAQREAALKRFRQKRQERCFEKKVRYQSRKKLAEQRPRIRGQFIRKGMNENKGKGINYEPEPIS

SEQ ID NO: 219 - protein, 394 aa, Cannabis sativa
MKMVQMNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIPVIMMSSHDSRSMVFKCLSKGAVDFLVKPIRKNELKNLWQHVWRKCHISSNSGSESGIWIEKPLKSRTVEHSDNNSGSNDEDDTDSIGLNFRNESDSGTQSSWTKRAEVDSPQAVSWEQFADLPDSTNHQVNHPRQEAFGNNWVPENATVTPRPHNDELDKKVMGKDLKIGLPSLLEDTSEKGLTNMEGTNKDKCSELNSKKDDQEQEKRELDLNNEEP

SEQ ID NO: 220 - DNA, 754 bp, Cannabis sativa
  source 1..754: mol_type = genomic DNA
ggcagacaagcttggaaagtcttagaagatcttgtgacagatgttgatctcgttttgactgaggtagtcatgccctgtttatctggtattggtcttctaggcaagatcatgagcaaaaaarcatgcaaggacatccctgtaattattgagtatatttcctttattttggatttaaaataatacttttttctctagtatctttttgtaatttatacttttaactaatacatttattgtgtgtgtttgtgtttgtgttttcacagtgatgtcttcacatgattctaggagtatggtctttaagtgtttatcgaaaggtgccgttgactttttagtgaaacctattcgaaagaatgagctgaaaaacctttggcaacatgtttggagaaaatgccacattgtgagttgcaatctatttgatttattatatcatggaccaattcaccttgaggttcaggtttcttctcatttatgcattttgtttttcattaagcaccaattgtcgaactataccaaagaaaacaaacaaaaaaatggcttcaatctttgcttgataaaaaatatgttttatgtaatgcaaacatcacttaattgacataataccattaaacacagaaatttgctcgagttgtgcattaatttttcatattttcatcagtctagtaatagtggaagtgaaagtggtatatggattgaaaagcctttaaagtcaagaactgtggaacattcagacaacaacagtggcagcaatgatgag

SEQ ID NO: 221 - DNA, 354 bp, Cannabis sativa
  source 1..354: mol_type = genomic DNA
tgttgatctcgttttgactgaggtagtcatgccctgtttatctggtattggtcttctaggcaagatcatgagcaaaaaaacatgcaaggacatccctgtaattattgagtatatttcctttattttggatttaaaataatacttttttctctagtatctttttgtaatttatacttttaactaatacatttattgtgtgtgtttgtgtttgtgttttcacagtgatgtcttcacatgattctaggagtatggtctttaagtgtttatcgaaaggtgccgttgactttttagtgaaacctattcgaaagaatgagctgaaaaacctttggcaacatgtttggagaaaatgccaca

SEQ ID NO: 222 - DNA, 340 bp, Cannabis sativa
  source 1..340: mol_type = genomic DNA
gttgatctcgttttgactgaggtagtcatgccctgtttatctggtattggtcttctaggcaagatcatgagcaaaaaaacatgcaaggacatccctgtaattagtgagtatatttcctttattttggatttaaaataatacttttttctctagtatctttttgtaatttatacttttaactaatacatttattgtgtgtgtttgtgtttgtgttttcacagtgatgtcttctcatgattctaggagtatggtctttaagtgtttatcgaaaggtgccgttgactttttagtgaaacctattcgaaagaatgagctgaaaaacctttggcaacatgtttgg

SEQ ID NO: 223 - DNA, 346 bp, Cannabis sativa
  source 1..346: mol_type = genomic DNA
tgttgatctcgttttgactgaggtagtcatgccctgtttatctggtattggtcttctaggcaagatcatgagcaaaaaaacatgcaaggacatccctgtaattagtgagtatatttcctttattttggatttaaaataatacttttttctctagtatctttttgtaatttatacttttaactaatacatttattgtgtgtgtttgtgtttgtgttttcacagtgatgtcttctcatgattctaggagtatggtctttaagtgtttatcgaaaggtgccgttgactttttagtgaaacctattcgaaagaatgagctgaaaaacctttggcaacatgtttggagaaa

SEQ ID NO: 224 - DNA, 4630 bp, Cannabis sativa
  source 1..4630: mol_type = genomic DNA
atgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaaggtcagtacgtttcagcaacatgaaatgatcattgcatttctcttatgcagaagtatacactctatgttaatggctttatgtttgattattgacggagatgacttttaaagtaaacactgtagtagatcttatgaataaacttgatgttatatgatatgaaaggaaaccatgtttatttgctccactacgagtaataatatccatggtctaatagttgttgtcactaaccataacctagaaagagtggtcttaggattagcctcgctgatccacacctcacctcttgtagagaaggtcaagggttcaatccctcccccgccctccaaagaaagatataacaaacagaaaacgaatcaaaagaataaaaaacaaacaagtaaggtgagcatggcactattattagactaaagcttgttttagtttaatataagtagatcttatttctgaaatgttattttccaccatctaattacacacatttctagactgatcccctgatcttgtggcagcgctctttttctgagttcctatcagaaatgctgaaaactattaaactagtttttgttacttatttattttcttttgttttaacactacattttaacaacagttcctatactctggtgcttcacgtgtctatttgtgctattttgatgttcatatttatagtctagcgggaagttttttagtcatttcgttcatgaagggtcaagtacgatttcttgacctagcttagattttgacatagaaccattcttgaggatactacagtgggttacttagtttgtagagtatgtttatgtgtaccttctaaagataacctggttataagtatgatattctcgaaaagaaaaacatgttgctccagacctgttggcaattgacatcttactttctagctatgataattagacattcagtcgctatatttatgtcatgctttcttcatcatcatttttcatatatgtgttcaagttatggtaagctatttaaatattgttattttatttgaccatgttttatttccaacgcaaactgcttggtataatattacataaatgttagccagaagctttgatttagtttacatcctaaaagtcaaaactagaattttattggttccctcaataaactaacaattattcaatgtattctcatattggcagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattagtgagtatatttcctttattttggatttaaaataatacttttttctctagtatctttttgtaatttatacttttaactaatacatttattgtgtgtgtttgtgtttgtgttttcacagtgatgtcttcacatgattctaggagtatggtctttaagtgtttatcgaaaggtgccgttgactttttagtgaaacctattcgaaagaatgagctgaaaaacctttggcaacatgtttggagaaaatgccacattgtgagttgcaatctatttgatttattatatcatggaccaattcaccttgaggttcaggtttcttctcatttatgcattttgtttttcattaagcaccaattgtcgaactataccaaagaaaacaaacaaaaaaatggcttcaatctttgcttgataaaaaatatgttttatgtaatgcaaacatcacttaattgacataataccattaaacacagaaatttgctcgagttgtgcattaatttttcatattttcatcagtctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacaggtatttcactaaatttcatgaaagagttttgttttttttttttggtgggaatgaagttttatgtctttgtatttacagaaatattagcgaaatgttagtttccacatgaagttttatgttttatgtatcaagaaactactataattatgttagtttccacacacctagtttggaatttgtctatcacagcacccatatatctatttaccatgtttgcatattactcatttgatcttggtggaggaatattcatgtaagagtttttaatacttttatgtatatgtaagtggagaaggaatgataattagcaagataagaaaacaagaaaaagaatgaaaacttactactgagcttttacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttggtaggcaaattttcctaacatcttttttttttttaactttcttccattctcgatcattctctaagttatgatttatttaaattgtttagacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtatagaaatattttgtgttgatataaacatgctcaataacaattaactctaaatttaagaatataactaagtcccattaccatgcaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaaggtaaatattcgaatatgatgcctaagtaattaattctaataagacacaagcaaaggccacataccacttcataataacttttcatcgatgttgatttgttttacaaaaactatggcagcagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttgaaaagattccgccagaagaggcaagaaagatgctttgagaaaaaggtaaacagaaaatcaccccccttattttctctaagaagtaaatcatggaaacaaacaagtaggcgtctgaaagaaggaagtttcattttcaacactacacatttgaaccatcatctttgggatccagttagtatgaagctttttgggaaaaaaaaaaggaaaagaacagaaaattgtttccctgaaatataaataattgatctgtttcctttgaagttccatattgctgacctgaagcattaatttttactttttcaggtgagatatcagagtagaaaaaaactggcagaacaaagaccccgaattcgaggacaatttattagaaagggaatgaatgaaaacaagggaaaaggcataaattacgaacctgaaccaatttcataa

SEQ ID NO: 225 - DNA, 51 bp, Cannabis sativa
  source 1..51: mol_type = genomic DNA
acatgcaaggacatccctgtaattagtgagtatatttcctttattttggat